SEQUENCE LISTING (81 sequences)

SEQ ID NO: 1 - protein, 7 aa, Artificial
  AAV pseudotype peptide sequence
GGMRRDP

SEQ ID NO: 2 - protein, 7 aa, Artificial
  AAV pseudotype peptide sequence
GVAISGF

SEQ ID NO: 3 - protein, 7 aa, Artificial
  AAV pseudotype peptide sequence
GQKWNVD

SEQ ID NO: 4 - protein, 7 aa, Artificial
  AAV pseudotype peptide sequence
GKVGVWA

SEQ ID NO: 5 - protein, 7 aa, Artificial
  AAV pseudotype peptide sequence
GAFELHN

SEQ ID NO: 6 - protein, 7 aa, Artificial
  AAV pseudotype peptide sequence
QVLRPTY

SEQ ID NO: 7 - protein, 7 aa, Artificial
  AAV pseudotype peptide sequence
LGRVHSL

SEQ ID NO: 8 - protein, 7 aa, Artificial
  AAV pseudotype peptide sequence
LYANVSP

SEQ ID NO: 9 - protein, 7 aa, Artificial
  AAV pseudotype peptide sequence
LSLGPTV

SEQ ID NO: 10 - protein, 7 aa, Artificial
  AAV pseudotype peptide sequence
LSLSLAT

SEQ ID NO: 11 - protein, 7 aa, Artificial
  AAV pseudotype peptide sequence
LNSSSYG

SEQ ID NO: 12 - protein, 7 aa, Artificial
  AAV pseudotype peptide sequence
LLRNSTA

SEQ ID NO: 13 - protein, 7 aa, Artificial
  AAV pseudotype peptide sequence
FHSSASA

SEQ ID NO: 14 - protein, 7 aa, Artificial
  AAV pseudotype peptide sequence
FVGDTVA

SEQ ID NO: 15 - protein, 7 aa, Artificial
  AAV pseudotype peptide sequence
FGRGSSG

SEQ ID NO: 16 - protein, 7 aa, Artificial
  AAV pseudotype peptide sequence
WDKAATF

SEQ ID NO: 17 - protein, 7 aa, Artificial
  AAV pseudotype peptide sequence
AEAPILL

SEQ ID NO: 18 - protein, 7 aa, Artificial
  AAV pseudotype peptide sequence
AFHGLLA

SEQ ID NO: 19 - protein, 7 aa, Artificial
  AAV pseudotype peptide sequence
AGYSIVH

SEQ ID NO: 20 - protein, 7 aa, Artificial
  AAV pseudotype peptide sequence
ATWNKSQ

SEQ ID NO: 21 - protein, 7 aa, Artificial
  AAV pseudotype peptide sequence
SVHSDYR

SEQ ID NO: 22 - protein, 7 aa, Artificial
  AAV pseudotype peptide sequence
SGRNSDL

SEQ ID NO: 23 - protein, 7 aa, Artificial
  AAV pseudotype peptide sequence
SVFVPMR

SEQ ID NO: 24 - protein, 7 aa, Artificial
  AAV pseudotype peptide sequence
VHGQYVE

SEQ ID NO: 25 - protein, 7 aa, Artificial
  AAV pseudotype peptide sequence
VERGASV

SEQ ID NO: 26 - protein, 7 aa, Artificial
  AAV pseudotype peptide sequence
VSEMKER

SEQ ID NO: 27 - protein, 7 aa, Artificial
  AAV pseudotype peptide sequence
GTPSHYG

SEQ ID NO: 28 - protein, 7 aa, Artificial
  AAV pseudotype peptide sequence
ILSKGAL

SEQ ID NO: 29 - protein, 7 aa, Artificial
  AAV pseudotype peptide sequence
GMGTVAR

SEQ ID NO: 30 - protein, 7 aa, Artificial
  AAV pseudotype peptide sequence
RVGHFSA

SEQ ID NO: 31 - protein, 7 aa, Artificial
  AAV pseudotype peptide sequence
PSMGFTP

SEQ ID NO: 32 - protein, 7 aa, Artificial
  AAV pseudotype peptide sequence
ALMTGKL

SEQ ID NO: 33 - protein, 7 aa, Artificial
  AAV pseudotype peptide sequence
PSMTRQT

SEQ ID NO: 34 - protein, 7 aa, Artificial
  AAV pseudotype peptide sequence
VERGASV

SEQ ID NO: 35 - protein, 7 aa, Artificial
  AAV pseudotype peptide sequence
KLIGRSP

SEQ ID NO: 36 - protein, 7 aa, Artificial
  AAV pseudotype peptide sequence
AEAPILL

SEQ ID NO: 37 - protein, 7 aa, Artificial
  AAV pseudotype peptide sequence
NPNLSLG

SEQ ID NO: 38 - protein, 7 aa, Artificial
  AAV pseudotype peptide sequence
TMKMFAS

SEQ ID NO: 39 - protein, 7 aa, Artificial
  AAV pseudotype peptide sequence
SAYNRAA

SEQ ID NO: 40 - protein, 7 aa, Artificial
  AAV pseudotype peptide sequence
IASTVYG

SEQ ID NO: 41 - protein, 7 aa, Artificial
  AAV pseudotype peptide sequence
VDSSRVR

SEQ ID NO: 42 - protein, 7 aa, Artificial
  AAV pseudotype peptide sequence
MNGLRGT

SEQ ID NO: 43 - protein, 7 aa, Artificial
  AAV pseudotype peptide sequence
YGNSKVV

SEQ ID NO: 44 - protein, 7 aa, Artificial
  AAV pseudotype peptide sequence
SAVYTRA

SEQ ID NO: 45 - protein, 7 aa, Artificial
  AAV pseudotype peptide sequence
LAITVAT

SEQ ID NO: 46 - protein, 7 aa, Artificial
  AAV pseudotype peptide sequence
FVKANVL

SEQ ID NO: 47 - protein, 7 aa, Artificial
  AAV pseudotype peptide sequence
GEDIGEH

SEQ ID NO: 48 - protein, 7 aa, Artificial
  AAV pseudotype peptide sequence
KSQLSYV

SEQ ID NO: 49 - protein, 7 aa, Artificial
  AAV pseudotype peptide sequence
ITLSGSS

SEQ ID NO: 50 - protein, 7 aa, Artificial
  AAV pseudotype peptide sequence
AKFPVKV

SEQ ID NO: 51 - protein, 7 aa, Artificial
  AAV pseudotype peptide sequence
SLTAMRG

SEQ ID NO: 52 - protein, 7 aa, Artificial
  AAV pseudotype peptide sequence
RSEYVRV

SEQ ID NO: 53 - protein, 7 aa, Artificial
  AAV pseudotype peptide sequence
VRDLTRV

SEQ ID NO: 54 - protein, 7 aa, Artificial
  AAV pseudotype peptide sequence
GIGHLAS

SEQ ID NO: 55 - protein, 7 aa, Artificial
  AAV pseudotype peptide sequence
HESRPPY

SEQ ID NO: 56 - protein, 7 aa, Artificial
  AAV pseudotype peptide sequence
SSTQKVS

SEQ ID NO: 57 - protein, 7 aa, Artificial
  AAV pseudotype peptide sequence
FGVSRST

SEQ ID NO: 58 - protein, 7 aa, Artificial
  AAV pseudotype peptide sequence
MIRGIPS

SEQ ID NO: 59 - protein, 7 aa, Artificial
  AAV pseudotype peptide sequence
IRQPTSH

SEQ ID NO: 60 - protein, 7 aa, Artificial
  AAV pseudotype peptide sequence
VSGAVLG

SEQ ID NO: 61 - protein, 7 aa, Artificial
  AAV pseudotype peptide sequence
RNSDNLS

SEQ ID NO: 62 - protein, 7 aa, Artificial
  AAV pseudotype peptide sequence
RLASFNS

SEQ ID NO: 63 - protein, 7 aa, Artificial
  AAV pseudotype peptide sequence
YRIGEGT

SEQ ID NO: 64 - protein, 7 aa, Artificial
  AAV pseudotype peptide sequence
VHSLGHM

SEQ ID NO: 65 - protein, 7 aa, Artificial
  AAV pseudotype peptide sequence
VTAVAKV

SEQ ID NO: 66 - protein, 7 aa, Artificial
  AAV pseudotype peptide sequence
LHHSPSY

SEQ ID NO: 67 - protein, 7 aa, Artificial
  AAV pseudotype peptide sequence
ERARSTN

SEQ ID NO: 68 - protein, 7 aa, Artificial
  AAV pseudotype peptide sequence
GMGLTVA

SEQ ID NO: 69 - protein, 7 aa, Artificial
  AAV pseudotype peptide sequence
LNSSSYG

SEQ ID NO: 70 - protein, 7 aa, Artificial
  AAV pseudotype peptide sequence
GYKQVSN

SEQ ID NO: 71 - protein, 7 aa, Artificial
  AAV pseudotype peptide sequence
GKASKDT

SEQ ID NO: 72 - protein, 7 aa, Artificial
  AAV pseudotype peptide sequence
QLSSRGA

SEQ ID NO: 73 - protein, 7 aa, Artificial
  AAV pseudotype peptide sequence
VGTRFTS

SEQ ID NO: 74 - protein, 7 aa, Artificial
  AAV pseudotype peptide sequence
QRSSNGP

SEQ ID NO: 75 - protein, 7 aa, Artificial
  AAV pseudotype peptide sequence
YKTQGLA

SEQ ID NO: 76 - protein, 7 aa, Artificial
  AAV pseudotype peptide sequence
RDSMVNP

SEQ ID NO: 77 - protein, 7 aa, Artificial
  AAV pseudotype peptide sequence
RVSSNLS

SEQ ID NO: 78 - protein, 7 aa, Artificial
  AAV pseudotype peptide sequence
DGTYKGA

SEQ ID NO: 79 - protein, 20 aa, Artificial
  AAV pseudotype peptide sequence
TGGATGATAACCAAGGTGAA

SEQ ID NO: 80 - protein, 26 aa, Artificial
  AAV pseudotype peptide sequence
TGGATGATAACCAGTGCCAAGGTGAA

SEQ ID NO: 81 - DNA, 21 bp, Artificial
  Nucleotide sequence encoding pseudotype insert
  misc_feature (1)..(2): n is a, c, g, or t
  misc_feature (4)..(5): n is a, c, g, or t
  misc_feature (7)..(8): n is a, c, g, or t
  misc_feature (10)..(11): n is a, c, g, or t
  misc_feature (13)..(14): n is a, c, g, or t
  misc_feature (16)..(17): n is a, c, g, or t
  misc_feature (19)..(20): n is a, c, g, or t
nnknnknnknnknnknnknnk

(19) United States
(12) Patent Application Publication
(10) Pub. No.: US 2024/0299586 A1
(43) Pub. Date: Sep. 12, 2024

(54) NOVEL AAV SEROTYPES DERIVED FROM A LIBRARY SCREEN

(71) Applicant: The Regents of the University of California, Oakland, CA (US)

(72) Inventors: Melissa J. Spencer, Los Angeles, CA (US); Michael Reza Emami, Diamond Bar, CA (US); April D. Pyle, Los Angeles, CA (US); Courtney S. Young, Los Angeles, CA (US); Donald B. Kohn, Tarzana, CA (US)

(73) Assignee: The Regents of the University of California, Oakland, CA (US)

(21) Appl. No.: 18/289,219

(22) PCT Filed: May 5, 2022
(86) PCT No.: PCT/US2022/027919

§ 371 Date: Nov. 1, 2023

Related U.S. Application Data
(60) Provisional application No. 63/185,194, filed on May 6, 2021.

(51) Int. Cl. A61K 48/00; C12N 9/22; C12N 15/11; C12N 15/86

(52) U.S. Cl. CPC A61K 48/0058; C12N 9/22; C12N 15/111; C12N 15/86; C12N 2310/20; C12N 2320/32; C12N 2750/14122; C12N 2750/14143; C12N 2750/14145; C12N 2750/14152

(57) ABSTRACT

In various embodiments, AAV serotypes capable of transducing skeletal muscle fibers and skeletal muscle stem cells (MuSC) with reduced off target tissue tropism are provided.

### CROSS-REFERENCE TO RELATED APPLICATIONS

This application claims priority to and benefit of U.S. Ser. No. 63/185,194, filed on May 6, 2021, which is incorporated herein by reference in its entirety for all purposes.

### STATEMENT OF GOVERNMENTAL SUPPORT

[Not Applicable]

### INCORPORATION BY REFERENCE OF SEQUENCE LISTING PROVIDED AS A TEXT FILE

This application contains references to nucleic acid sequences that have been submitted concurrently herewith as the sequence listing text file “UCLA-P235PUS_ST25.txt”, file size 15,737 bytes, created on May 5, 2021, which is incorporated by reference in its entirety pursuant to 37 C.F.R. § 1.52(e)(5).

### BACKGROUND

Clinical gene transfer utilizing adeno-associated viral (AAV) vectors has rapidly gained momentum in recent years. The ongoing translation from bench-to-bedside has been spurred in part by the availability of a wide variety of AAV displaying diverse tissue tropisms across multiple species (see, e.g., Herzog et al. (2010) Discov. Med. 9: 105-111; Mitchell et al. (2010) Curr. Gene Ther. 10: 319-340). Among numerous AAV isolates, AAV9 vectors display a systemic, multiorgan transduction profile following intravenous administration (see, e.g., Zincarelli et al. (2008) Mol. Ther. 16: 1073-1080). Rapid onset of gene expression and high transgene expression levels mediated by AAV9 vectors in heart and liver have been reported (see, e.g., Inagaki et al. (2006) Mol. Ther. 14: 45-53; Pacak et al. (2006) Circ. Res. 99: e3-e9; Yang et al. (2009) Proc. Natl. Acad. Sci. USA, 106: 3946-3951).

A wide range of clinically relevant applications require vector targeting to specific tissues rather than multiorgan gene expression. For instance, gene therapy of cardiac disease or muscular dystrophies would be greatly facilitated by vectors capable of efficient and selective gene transfer to heart and/or skeletal muscle, and/or muscle stem cells (see, e.g., Yang et al. (2009) Proc. Natl. Acad. Sci. USA, 106: 3946-3951; Kornegay et al. (2010) Mol. Ther. 18: 1501-1508; Lipskaia et al. (2010) Expert Opin. Biol. Ther. 10: 29-41).

Tissue-specific transgene expression utilizing AAV vectors has been achieved through different approaches. For instance, at the vector genome level, transcriptional targeting elements such as tissue-specific promoters (see, e.g., Salva et al. (2007) Mol. Ther. 15: 320-329; Wang et al. (2008) Gene Ther. 15: 1489-1499) and microRNA-mediated gene regulation (see, e.g., Brown & Naldini (2009) Nat. Rev. Genet. 10: 578-585) have been developed. When used in conjunction with the appropriate AAV serotype or variant, the latter strategies can provide precise control over gene expression patterns in different tissues following systemic injection.

Attempts to develop tissue-targeted AAV vectors have included insertion of targeting motifs into the GH loop of VP3 subunits of different AAV capsid templates or directed evolution of tissue-tropic AAV variants through DNA shuffling (see, e.g., Michelfelder et al. (2009) PLoS ONE, 4: e5122; Ying et al. (2010) Gene Ther. 17: 980-990; Excoffon et al. (2009) Proc. Natl. Acad. Sci. USA, 106: 3865-3870; Koerber et al. (2009) Mol. Ther. 17: 2088-2095.

However, the development of AAV vectors for specific or preferential delivery of nucleic acid constructs (e.g., CRISPR/Cas, transgenes, etc.) various muscle types (e.g., to skeletal muscle or to muscle stem cells), while sparing cardiac muscle and/or liver have proven elusive.

### SUMMARY

Described herein are AAV serotypes that have unique tissue tropism. In particular, AAV serotypes capable of transducing skeletal muscle fibers and skeletal muscle stem cells (MuSC) with reduced off target tissue tropism are provided. In certain embodiments illustrative, but non-limiting embodiments, transducing MuSCs can greatly improve the efficacy of our CRISPR platform described in PCT Publication No: PCT/US2017/017255, and other CRISPR platforms or gene therapy platforms. Thus, MuSC targeting is a priority.

There are several claims in the literature that AAV serotype 9 can transduce muscle stem cells. We tested this claim and could not reproduce the findings thus we sought to generate new AAV serotypes with the capacity to target MuSCs and other tissues of the body (e.g., heart and skeletal muscles).

Accordingly, various embodiments provided herein may include, but need not be limited to, one or more of the following:

Various embodiments provided herein may include, but need not be limited to, one or more of the following:

Embodiment 1: An adeno-associated virus (AAV) vector pseudotyped to transfect muscle and/or muscle stem cells, wherein said vector comprises a capsid protein comprising a peptide insert that improves the ability of said vector to transduce a muscle cell and/or a muscle stem cell.

Embodiment 2: The AAV vector of embodiment 1, wherein said peptide insert ranges in length from about 7 up to about 15 amino acids.

Embodiment 3: The AAV vector of embodiment 2, wherein said peptide insert ranges in length from about 7 up to about 10 amino acids, or up to about 9 amino acids.

Embodiment 4: The AAV vector of embodiment 3, wherein said peptide insert has a length of seven amino acids.

Embodiment 5: The AAV vector according to any one of embodiments 1-4, wherein said peptide insert comprises or consists of one or more amino acid sequences independently selected from the group consisting of GGMRRDP (SEQ ID NO:1), GVAISGF (SEQ ID NO:2), GQKWNVD (SEQ ID NO:3), GKVGVWA (SEQ ID NO:4), GAFELHN (SEQ ID NO:5), QVLRPTY (SEQ ID NO:6), LGRVHSL (SEQ ID NO:7), LYANVSP (SEQ ID NO:8), LSLGPTV (SEQ ID NO:9), LSLSLAT (SEQ ID NO:10), LNSSSYG (SEQ ID NO:11), LLRNSTA (SEQ ID NO:12), FHSSASA (SEQ ID NO:13), FVGDTVA (SEQ ID NO:14), FGRGSSG (SEQ ID NO:15), WDKAATF (SEQ ID NO:16), AEAPILL (SEQ ID NO:17), AFHGLLA (SEQ ID NO:18), AGYSIVH (SEQ ID NO:19), ATWNKSQ (SEQ ID NO:20), SVHSDYR (SEQ ID NO:21), SGRNSDL (SEQ ID NO:22), SVFVPMR (SEQ ID NO:23), VHGQYVE (SEQ ID NO:24), VERGASV (SEQ ID NO:25), VSEMKER (SEQ ID NO:26), INHNSHI (SEQ ID NO:27), GQSNISR (SEQ ID NO:28), RQTNQIE (SEQ ID NO:29), GPVVQGA (SEQ ID NO:30), VSGSSFG (SEQ ID NO:31), DVSLTRM (SEQ ID NO:32), NGQGILK (SEQ ID NO:33), SGDAYRA (SEQ ID NO:34), FNNGIHS (SEQ ID NO:35), SSYSVSL (SEQ ID NO:36), ESGNIRP (SEQ ID NO:37), VSGGQTQ (SEQ ID NO:38), IVGSGSA (SEQ ID NO:39), VSSQGTS (SEQ ID NO:40), GSTYENS (SEQ ID NO:41), NGSTSPV (SEQ ID NO:42), EGKSSPT (SEQ ID NO:43), VGQGISL (SEQ ID NO:44), AGIGQAG (SEQ ID NO:45), ITSPSVS (SEQ ID NO:46), EHVEGRR (SEQ ID NO:47), LDGVSHM (SEQ ID NO:48), GGGTLHH (SEQ ID NO:49), HESRPPY (SEQ ID NO:50), SNHDSRV (SEQ ID NO:51), SGSGSFM (SEQ ID NO:52), VGSLYQP (SEQ ID NO:53), GQDNRAL (SEQ ID NO:54), RSEYNSV (SEQ ID NO:55), TGQTVLG (SEQ ID NO:56), GGDAARY (SEQ ID NO:57), RGSNDLP (SEQ ID NO:58), VASGTVL (SEQ ID NO:59), GDHNTIK (SEQ ID NO:60), NARDSGF (SEQ ID NO:61), PGNINNN (SEQ ID NO:62), SCGDSCR (SEQ ID NO:63), FGSVHSP (SEQ ID NO:64), SLGGLGN (SEQ ID NO:65), AADAVRV (SEQ ID NO:66), VIAQGMS (SEQ ID NO:67), NHSSGAM (SEQ ID NO:68), LRDNSAF (SEQ ID NO:69), LGSGALA (SEQ ID NO:70), SHEVVRA (SEQ ID NO:71), FNISANE (SEQ ID NO:72), ARSQEVP (SEQ ID NO:73), AGNITSF (SEQ ID NO:74), TCNSVGC (SEQ ID NO:75), GGGSSSA (SEQ ID NO:76), YDQVSHP (SEQ ID NO:77), VHGISPL (SEQ ID NO:78), TGLNYTT (SEQ ID NO:79), QDRNYGV (SEQ ID NO:80), VSEINRG (SEQ ID NO:81), QRSSNGP (SEQ ID NO:82), SSTGTWV (SEQ ID NO:83), QLPNNLS (SEQ ID NO:84), YNGAIHH (SEQ ID NO:85), MRNTESS (SEQ ID NO:86), GASPSSI (SEQ ID NO:87), DGTYKGA (SEQ ID NO:88), GQGCTQC (SEQ ID NO:89), GAHPTGG (SEQ ID NO:90), SNYNVTG (SEQ ID NO:91), SANVFSP (SEQ ID NO:92), AHSGGTY (SEQ ID NO:93), GENAGRA (SEQ ID NO:94), IGCDYPC (SEQ ID NO:95), ISLPNSI (SEQ ID NO:96), NGINGAS (SEQ ID NO:97), GPSSLRE (SEQ ID NO:98), VQSGNYV (SEQ ID NO:99), GGYSETS (SEQ ID NO:100), LRVGNVE (SEQ ID NO:101), GMGTVAR (SEQ ID NO:102), VQMSGYL (SEQ ID NO:103), QNMVVTS (SEQ ID NO:104), THGLPIV (SEQ ID NO:105), GFNHGAN (SEQ ID NO:106), NSYSVSP (SEQ ID NO:107), SMLGGVA (SEQ ID NO:108), YGVDMGL (SEQ ID NO:109), VIGHGSA (SEQ ID NO:110), SGLDKAP (SEQ ID NO:111), DVKNGGR (SEQ ID NO:112), IGNGGLI (SEQ ID NO: 113), QVGFPTP (SEQ ID NO: 114), LSSNANL (SEQ ID NO:115), TNLHVNL (SEQ ID NO: 116), SYIGVAA (SEQ ID NO: 117), STVNGNQ (SEQ ID NO:118), TNRDAQF (SEQ ID NO:119), LHHSPSY (SEQ ID NO:120), TTYNSSA (SEQ ID NO:121), PSSQPLP (SEQ ID NO:122), RDSMVNP (SEQ ID NO:123), SLHSPQL (SEQ ID NO:124), VQVGTGT (SEQ ID NO:125), RENPSGM (SEQ ID NO:126), GVPKTHD (SEQ ID NO:127), VDQGRLS (SEQ ID NO:128), FDKGAVI (SEQ ID NO:129), VSRSGGV (SEQ ID NO:130), INMQSMA (SEQ ID NO:131), GTASSRP (SEQ ID NO:132), EVGHIRG (SEQ ID NO:133), RDVSSGG (SEQ ID NO:134), ITLSGSS (SEQ ID NO:135), MNGLRGT (SEQ ID NO:136), RSQEINL (SEQ ID NO:137), YGTVSVP (SEQ ID NO:138), LGSSGVS (SEQ ID NO:139), PGSYVSS (SEQ ID NO:140), ASNLGTY (SEQ ID NO:141), GSEGGSM (SEQ ID NO:142), IASGNSV (SEQ ID NO:143), ATAGAII (SEQ ID NO:144), LSSMGQL (SEQ ID NO:145), GEKFMGV (SEQ ID NO:146), GTPSHYG (SEQ ID NO:147), ILSKGAL (SEQ ID NO:148), GMGTVAR (SEQ ID NO:149), RVGHFSA (SEQ ID NO:150), PSMGFTP (SEQ ID NO:151), ALMTGKL (SEQ ID NO:152), PSMTRQT (SEQ ID NO:153), VERGASV (SEQ ID NO:154), KLIGRSP (SEQ ID NO:155), AEAPILL (SEQ ID NO:156), NPNLSLG (SEQ ID NO:157), TMKMFAS (SEQ ID NO:158), SAYNRAA (SEQ ID NO:159), IASTVYG (SEQ ID NO:160), VDSSRVR (SEQ ID NO:161), MNGLRGT (SEQ ID NO:162), YGNSKVV (SEQ ID NO:163), SAVYTRA (SEQ ID NO:164), LAITVAT (SEQ ID NO:165), FVKANVL (SEQ ID NO:166), GEDIGEH (SEQ ID NO:167), KSQLSYV (SEQ ID NO:168), ITLSGSS (SEQ ID NO:169), AKFPVKV (SEQ ID NO:170), SLTAMRG (SEQ ID NO:171), RSEYVRV (SEQ ID NO:172), WNGELVA (SEQ ID NO:173), GQSNISR (SEQ ID NO:174), AEAPILL (SEQ ID NO:175), YGTVSVP (SEQ ID NO:176), GLLNSSP (SEQ ID NO:177), YESSRTA (SEQ ID NO:178), ESGNIRP (SEQ ID NO:179), LNSSSYG (SEQ ID NO:180), DNRTVNV (SEQ ID NO:181), SGSGSFM (SEQ ID NO:182), TRLNTVQ (SEQ ID NO:183), VSGGQTQ (SEQ ID NO:184), DGTYKGA (SEQ ID NO:185), RQTNQIE (SEQ ID NO:186), GTASSRP (SEQ ID NO:187), LGSSGVS (SEQ ID NO:188), AHSGGTY (SEQ ID NO:189), SHEVVRA (SEQ ID NO:190), YISESRP (SEQ ID NO:191), SLGGLGN (SEQ ID NO:192), NSYSVSP (SEQ ID NO:193), GSITYTP (SEQ ID NO:194), NGINGAS (SEQ ID NO:195), VSGSSFG (SEQ ID NO:196), EVRVANA (SEQ ID NO:197), SGRNSDL (SEQ ID NO:198), MTGLRGT (SEQ ID NO:199), GFNHGAN (SEQ ID NO:200), GENAGRA (SEQ ID NO:201), QNMVVTS (SEQ ID NO:202), GASPSSI (SEQ ID NO:203), YVNKSQA (SEQ ID NO:204), GTPSQYG (SEQ ID NO:205), NHSSGAM (SEQ ID NO:206), TNLNLRE (SEQ ID NO:207), GRISGGS (SEQ ID NO:208), VRDLTRV (SEQ ID NO:209), GIGHLAS (SEQ ID NO:210), HESRPPY (SEQ ID NO:211), SSTQKVS (SEQ ID NO:212), FGVSRST (SEQ ID NO:213), MIRGIPS (SEQ ID NO:214), IRQPTSH (SEQ ID NO:215), VSGAVLG (SEQ ID NO:216), RNSDNLS (SEQ ID NO:217), RLASFNS (SEQ ID NO:218), YRIGEGT (SEQ ID NO:219), VHSLGHM (SEQ ID NO:220), VTAVAKV (SEQ ID NO:221), LHHSPSY (SEQ ID NO:222), ERARSTN (SEQ ID NO:223), GMGLTVA (SEQ ID NO:224), LNSSSYG (SEQ ID NO:225), GYKQVSN (SEQ ID NO:226), GKASKDT (SEQ ID NO:227), QLSSRGA (SEQ ID NO:228), VGTRFTS (SEQ ID NO:229), QRSSNGP (SEQ ID NO:230), YKTQGLA (SEQ ID NO:231), RDSMVNP (SEQ ID NO:232), RVSSNLS (SEQ ID NO:233), DGTYKGA (SEQ ID NO:234), FGSGALA (SEQ ID NO:235), ARSQEVP (SEQ ID NO:236), VVVVGRP (SEQ ID NO:237), LHGARSS (SEQ ID NO:238), SGQMRGA (SEQ ID NO:239), TRLNTVQ (SEQ ID NO:240), HMHVCVP (SEQ ID NO:241), VTNSSRV (SEQ ID NO:242), NQLPHFK (SEQ ID NO:243), VRFGSNP (SEQ ID NO:244), YISESRP (SEQ ID NO:245), VSRSGGV (SEQ ID NO:246), LSSNANL (SEQ ID NO:247), DNRTVNV (SEQ ID NO:248), LRVGNVE (SEQ ID NO:249), GTASSRP (SEQ ID NO:250), LHGARYS (SEQ ID NO:251), TGVNVKS (SEQ ID NO:252), VTSWTKN (SEQ ID NO:253), RGSTMMP (SEQ ID NO:254), SGSGSFM (SEQ ID NO:255), VSGGQTQ (SEQ ID NO:256), GMGTVAR (SEQ ID NO:257), LNPRSSS (SEQ ID NO:258), SQNLLSN (SEQ ID NO:259), YRIGEGT (SEQ ID NO:260), STVNGNQ (SEQ ID NO:261), AADAVRV (SEQ ID NO:262), YGNSKVV (SEQ ID NO:263), VPGEGRV (SEQ ID NO:264), GGDAARY (SEQ ID NO:265), YDQVSHP (SEQ ID NO:266), GSFHGTV (SEQ ID NO:267), GAHPTGG (SEQ ID NO:268), RDGQSRG (SEQ ID NO:269), SLGGLGN (SEQ ID NO:270), DGTYKGA (SEQ ID NO:271), GIGHLAS (SEQ ID NO:272), IGSVGKL (SEQ ID NO:273), SHEVVRA (SEQ ID NO:274), EVGHIRG (SEQ ID NO:275), and GLRGVSV (SEQ ID NO:276).

Embodiment 6: The AAV vector of embodiment 5, wherein said peptide insert comprises or consists of an amino acid sequence selected from the group consisting of

(SEQ ID NO: 1)
GGMRRDP,
(SEQ ID NO: 2)
GVAISGF,
(SEQ ID NO: 3)
GQKWNVD,
(SEQ ID NO: 4)
GKVGVWA,
(SEQ ID NO: 5)
GAFELHN,
(SEQ ID NO: 6)
QVLRPTY,
(SEQ ID NO: 7)
LGRVHSL,
(SEQ ID NO: 8)
LYANVSP,
(SEQ ID NO: 9)
LSLGPTV,
(SEQ ID NO: 10)
LSLSLAT,
(SEQ ID NO: 11)
LNSSSYG,
(SEQ ID NO: 12)
LLRNSTA,
(SEQ ID NO: 13)
FHSSASA,
(SEQ ID NO: 14)
FVGDTVA,
(SEQ ID NO: 15)
FGRGSSG,
(SEQ ID NO: 16)
WDKAATF,
(SEQ ID NO: 17)
AEAPILL,
(SEQ ID NO: 18)
AFHGLLA,
(SEQ ID NO: 19)
AGYSIVH,
(SEQ ID NO: 20)
ATWNKSQ,
(SEQ ID NO: 21)
SVHSDYR,
(SEQ ID NO: 22)
SGRNSDL,
(SEQ ID NO: 23)
SVFVPMR,
(SEQ ID NO: 24)
VHGQYVE,
(SEQ ID NO: 25)
VERGASV,
(SEQ ID NO: 26)
VSEMKER,
(SEQ ID NO: 27)
GTPSHYG,
(SEQ ID NO: 28)
ILSKGAL,
(SEQ ID NO: 29)
GMGTVAR,
(SEQ ID NO: 30)
RVGHFSA,
(SEQ ID NO: 31)
PSMGFTP,
(SEQ ID NO: 32)
ALMTGKL,
(SEQ ID NO: 33)
PSMTRQT,
(SEQ ID NO: 34)
VERGASV,
(SEQ ID NO: 35)
KLIGRSP,
(SEQ ID NO: 36)
AEAPILL,
(SEQ ID NO: 37)
NPNLSLG,
(SEQ ID NO: 38)
TMKMFAS,
(SEQ ID NO: 39)
SAYNRAA,
(SEQ ID NO: 40)
IASTVYG,
(SEQ ID NO: 41)
VDSSRVR,
(SEQ ID NO: 42)
MNGLRGT,
(SEQ ID NO: 43)
YGNSKVV,
(SEQ ID NO: 44)
SAVYTRA,
(SEQ ID NO: 45)
LAITVAT,
(SEQ ID NO: 46)
FVKANVL,
(SEQ ID NO: 47)
GEDIGEH,
(SEQ ID NO: 48)
KSQLSYV,
(SEQ ID NO: 49)
ITLSGSS,
(SEQ ID NO: 50)
AKFPVKV,
(SEQ ID NO: 51)
SLTAMRG,
(SEQ ID NO: 52)
RSEYVRV,
(SEQ ID NO: 53)
VRDLTRV,
(SEQ ID NO: 54)
GIGHLAS,
(SEQ ID NO: 55)
HESRPPY,
(SEQ ID NO: 56)
SSTQKVS,
(SEQ ID NO: 57)
FGVSRST,
(SEQ ID NO: 58)
MIRGIPS,
(SEQ ID NO: 59)
IRQPTSH,
(SEQ ID NO: 60)
VSGAVLG,
(SEQ ID NO: 61)
RNSDNLS,
(SEQ ID NO: 62)
RLASENS,
(SEQ ID NO: 63)
YRIGEGT,
(SEQ ID NO: 64)
VHSLGHM,
(SEQ ID NO: 65)
VTAVAKV,
(SEQ ID NO: 66)
LHHSPSY,
and
(SEQ ID NO: 67)
ERARSTN

Embodiment 7: The AAV vector of embodiment 5, wherein said vector transduces heart muscle and said peptide insert comprises or consists of one or more amino acid sequences selected from the group consisting of GGMRRDP (SEQ ID NO:1), GVAISGF (SEQ ID NO:2), GQKWNVD (SEQ ID NO:3), GKVGVWA (SEQ ID NO:4), GAFELHN (SEQ ID NO:5), QVLRPTY (SEQ ID NO:6), LGRVHSL (SEQ ID NO:7), LYANVSP (SEQ ID NO:8), LSLGPTV (SEQ ID NO:9), LSLSLAT (SEQ ID NO:10), LNSSSYG (SEQ ID NO: 11), LLRNSTA (SEQ ID NO:12), FHSSASA (SEQ ID NO:13), FVGDTVA (SEQ ID NO:14), FGRGSSG (SEQ ID NO:15), WDKAATF (SEQ ID NO:16), AEAPILL (SEQ ID NO:17), AFHGLLA (SEQ ID NO:18), AGYSIVH (SEQ ID NO:19), ATWNKSQ (SEQ ID NO:20), SVHSDYR (SEQ ID NO:21), SGRNSDL (SEQ ID NO:22), SVFVPMR (SEQ ID NO:23), VHGQYVE (SEQ ID NO:24), VERGASV (SEQ ID NO:25), VSEMKER (SEQ ID NO:26), INHNSHI (SEQ ID NO:27), GQSNISR (SEQ ID NO:28), RQTNQIE (SEQ ID NO:29), GPVVQGA (SEQ ID NO:30), VSGSSFG (SEQ ID NO:31), DVSLTRM (SEQ ID NO:32), NGQGILK (SEQ ID NO:33), SGDAYRA (SEQ ID NO:34), FNNGIHS (SEQ ID NO:35), SSYSVSL (SEQ ID NO:36), ESGNIRP (SEQ ID NO:37), VSGGQTQ (SEQ ID NO:38), IVGSGSA (SEQ ID NO:39), VSSQGTS (SEQ ID NO:40), GSTYENS (SEQ ID NO:41), NGSTSPV (SEQ ID NO:42), EGKSSPT (SEQ ID NO:43), VGQGISL (SEQ ID NO:44), AGIGQAG (SEQ ID NO:45), ITSPSVS (SEQ ID NO:46), EHVEGRR (SEQ ID NO:47), LDGVSHM (SEQ ID NO:48), GGGTLHH (SEQ ID NO:49), HESRPPY (SEQ ID NO:50), SNHDSRV (SEQ ID NO:51), SGSGSFM (SEQ ID NO:52), VGSLYQP (SEQ ID NO:53), GQDNRAL (SEQ ID NO:54), RSEYNSV (SEQ ID NO:55), TGQTVLG (SEQ ID NO:56), GGDAARY (SEQ ID NO:57), RGSNDLP (SEQ ID NO:58), VASGTVL (SEQ ID NO:59), GDHNTIK (SEQ ID NO:60), NARDSGF (SEQ ID NO:61), PGNINNN (SEQ ID NO:62), SCGDSCR (SEQ ID NO:63), FGSVHSP (SEQ ID NO:64), SLGGLGN (SEQ ID NO:65), AADAVRV (SEQ ID NO:66), VIAQGMS (SEQ ID NO:67), NHSSGAM (SEQ ID NO:68), LRDNSAF (SEQ ID NO:69), LGSGALA (SEQ ID NO:70), SHEVVRA (SEQ ID NO:71), FNISANE (SEQ ID NO:72), ARSQEVP (SEQ ID NO:73), AGNITSF (SEQ ID NO:74), TCNSVGC (SEQ ID NO:75), GGGSSSA (SEQ ID NO:76), YDQVSHP (SEQ ID NO:77), VHGISPL (SEQ ID NO:78), TGLNYTT (SEQ ID NO:79), QDRNYGV (SEQ ID NO:80), VSEINRG (SEQ ID NO:81), QRSSNGP (SEQ ID NO:82), SSTGTWV (SEQ ID NO:83), QLPNNLS (SEQ ID NO:84), YNGAIHH (SEQ ID NO:85), MRNTESS (SEQ ID NO:86), GASPSSI (SEQ ID NO:87), DGTYKGA (SEQ ID NO:88), GQGCTQC (SEQ ID NO:89), GAHPTGG (SEQ ID NO:90), SNYNVTG (SEQ ID NO:91), SANVFSP (SEQ ID NO:92), AHSGGTY (SEQ ID NO:93), GENAGRA (SEQ ID NO:94), IGCDYPC (SEQ ID NO:95), ISLPNSI (SEQ ID NO:96), NGINGAS (SEQ ID NO:97), GPSSLRE (SEQ ID NO:98), VQSGNYV (SEQ ID NO:99), GGYSETS (SEQ ID NO:100), LRVGNVE (SEQ ID NO:101), GMGTVAR (SEQ ID NO:102), VQMSGYL (SEQ ID NO:103), QNMVVTS (SEQ ID NO:104), THGLPIV (SEQ ID NO:105), GFNHGAN (SEQ ID NO:106), NSYSVSP (SEQ ID NO:107), SMLGGVA (SEQ ID NO:108), YGVDMGL (SEQ ID NO:109), VIGHGSA (SEQ ID NO:110), SGLDKAP (SEQ ID NO:111), DVKNGGR (SEQ ID NO:112), IGNGGLI (SEQ ID NO: 113), QVGFPTP (SEQ ID NO: 114), LSSNANL (SEQ ID NO:115), TNLHVNL (SEQ ID NO:116), SYIGVAA (SEQ ID NO:117), STVNGNQ (SEQ ID NO:118), TNRDAQF (SEQ ID NO:119), LHHSPSY (SEQ ID NO:120), TTYNSSA (SEQ ID NO:121), PSSQPLP (SEQ ID NO:122), RDSMVNP (SEQ ID NO:123), SLHSPQL (SEQ ID NO:124), VQVGTGT (SEQ ID NO:125), RENPSGM (SEQ ID NO:126), GVPKTHD (SEQ ID NO:127), VDQGRLS (SEQ ID NO:128), FDKGAVI (SEQ ID NO:129), VSRSGGV (SEQ ID NO:130), INMQSMA (SEQ ID NO:131), GTASSRP (SEQ ID NO:132), EVGHIRG (SEQ ID NO:133), RDVSSGG (SEQ ID NO:134), ITLSGSS (SEQ ID NO:135), MNGLRGT (SEQ ID NO:136), RSQEINL (SEQ ID NO:137), YGTVSVP (SEQ ID NO:138), LGSSGVS (SEQ ID NO:139), PGSYVSS (SEQ ID NO:140), ASNLGTY (SEQ ID NO:141), GSEGGSM (SEQ ID NO:142), IASGNSV (SEQ ID NO:143), ATAGAII (SEQ ID NO:144), LSSMGQL (SEQ ID NO:145), and GEKFMGV (SEQ ID NO:146).

Embodiment 8: The AAV vector of embodiment 7, wherein said vector transduces heart muscle and said peptide insert comprises or consists of one or more amino acid sequences selected from the group consisting of GGMRRDP (SEQ ID NO:1), GVAISGF (SEQ ID NO:2), GQKWNVD (SEQ ID NO:3), GKVGVWA (SEQ ID NO:4), GAFELHN (SEQ ID NO:5), QVLRPTY (SEQ ID NO:6), LGRVHSL (SEQ ID NO:7), LYANVSP (SEQ ID NO:8), LSLGPTV (SEQ ID NO:9), LSLSLAT (SEQ ID NO:10), LNSSSYG (SEQ ID NO: 11), LLRNSTA (SEQ ID NO:12), FHSSASA (SEQ ID NO:13), FVGDTVA (SEQ ID NO:14), FGRGSSG (SEQ ID NO:15), WDKAATF (SEQ ID NO:16), AEAPILL (SEQ ID NO:17), AFHGLLA (SEQ ID NO:18), AGYSIVH (SEQ ID NO:19), ATWNKSQ (SEQ ID NO:20), SVHSDYR (SEQ ID NO:21), SGRNSDL (SEQ ID NO:22), SVFVPMR (SEQ ID NO:23), VHGQYVE (SEQ ID NO:24), VERGASV (SEQ ID NO:25), and VSEMKER (SEQ ID NO:26).

Embodiment 9: The AAV vector of embodiment 7, wherein said vector transduces heart muscle and said peptide insert comprises or consists of one or more amino acid sequences selected from the group consisting of INHNSHI (SEQ ID NO:27), GQSNISR (SEQ ID NO:28), RQTNQIE (SEQ ID NO:29), GPVVQGA (SEQ ID NO:30), VSGSSFG (SEQ ID NO:31), DVSLTRM (SEQ ID NO:32), NGQGILK (SEQ ID NO:33), SGDAYRA (SEQ ID NO:34), FNNGIHS (SEQ ID NO:35), SSYSVSL (SEQ ID NO:36), ESGNIRP (SEQ ID NO:37), VSGGQTQ (SEQ ID NO:38), IVGSGSA (SEQ ID NO:39), VSSQGTS (SEQ ID NO:40), GSTYENS (SEQ ID NO:41), NGSTSPV (SEQ ID NO:42), EGKSSPT (SEQ ID NO:43), VGQGISL (SEQ ID NO:44), AGIGQAG (SEQ ID NO:45), ITSPSVS (SEQ ID NO:46), EHVEGRR (SEQ ID NO:47), LDGVSHM (SEQ ID NO:48), GGGTLHH (SEQ ID NO:49), HESRPPY (SEQ ID NO:50), SNHDSRV (SEQ ID NO:51), SGSGSFM (SEQ ID NO:52), VGSLYQP (SEQ ID NO:53), GQDNRAL (SEQ ID NO:54), RSEYNSV (SEQ ID NO:55), TGQTVLG (SEQ ID NO:56), GGDAARY (SEQ ID NO:57), RGSNDLP (SEQ ID NO:58), VASGTVL (SEQ ID NO:59), GDHNTIK (SEQ ID NO:60), NARDSGF (SEQ ID NO:61), PGNINNN (SEQ ID NO:62), SCGDSCR (SEQ ID NO:63), FGSVHSP (SEQ ID NO:64), SLGGLGN (SEQ ID NO:65), AADAVRV (SEQ ID NO:66), VIAQGMS (SEQ ID NO:67), NHSSGAM (SEQ ID NO:68), LRDNSAF (SEQ ID NO:69), LGSGALA (SEQ ID NO:70), SHEVVRA (SEQ ID NO:71), FNISANE (SEQ ID NO:72), ARSQEVP (SEQ ID NO:73), AGNITSF (SEQ ID NO:74), TCNSVGC (SEQ ID NO:75), GGGSSSA (SEQ ID NO:76), YDQVSHP (SEQ ID NO:77), VHGISPL (SEQ ID NO:78), TGLNYTT (SEQ ID NO:79), QDRNYGV (SEQ ID NO:80), VSEINRG (SEQ ID NO:81), QRSSNGP (SEQ ID NO:82), SSTGTWV (SEQ ID NO:83), QLPNNLS (SEQ ID NO:84), YNGAIHH (SEQ ID NO:85), MRNTESS (SEQ ID NO:86), GASPSSI (SEQ ID NO:87), DGTYKGA (SEQ ID NO:88), GQGCTQC (SEQ ID NO:89), GAHPTGG (SEQ ID NO:90), SNYNVTG (SEQ ID NO:91), SANVFSP (SEQ ID NO:92), AHSGGTY (SEQ ID NO:93), GENAGRA (SEQ ID NO:94), IGCDYPC (SEQ ID NO:95), ISLPNSI (SEQ ID NO:96), NGINGAS (SEQ ID NO:97), GPSSLRE (SEQ ID NO:98), VQSGNYV (SEQ ID NO:99), GGYSETS (SEQ ID NO:100), LRVGNVE (SEQ ID NO:101), GMGTVAR (SEQ ID NO:102), VQMSGYL (SEQ ID NO:103), QNMVVTS (SEQ ID NO:104), THGLPIV (SEQ ID NO:105), GFNHGAN (SEQ ID NO:106), NSYSVSP (SEQ ID NO:107), SMLGGVA (SEQ ID NO:108), YGVDMGL (SEQ ID NO:109), VIGHGSA (SEQ ID NO:110), SGLDKAP (SEQ ID NO:111), DVKNGGR (SEQ ID NO:112), IGNGGLI (SEQ ID NO:113), QVGFPTP (SEQ ID NO:114), LSSNANL (SEQ ID NO:115), TNLHVNL (SEQ ID NO:116), SYIGVAA (SEQ ID NO:117), STVNGNQ (SEQ ID NO: 118), TNRDAQF (SEQ ID NO: 119), LHHSPSY (SEQ ID NO:120), TTYNSSA (SEQ ID NO:121), PSSQPLP (SEQ ID NO:122), RDSMVNP (SEQ ID NO:123), SLHSPQL (SEQ ID NO:124), VQVGTGT (SEQ ID NO:125), RENPSGM (SEQ ID NO:126), GVPKTHD (SEQ ID NO:127), VDQGRLS (SEQ ID NO:128), FDKGAVI (SEQ ID NO:129), VSRSGGV (SEQ ID NO:130), INMQSMA (SEQ ID NO:131), GTASSRP (SEQ ID NO:132), EVGHIRG (SEQ ID NO:133), RDVSSGG (SEQ ID NO:134), ITLSGSS (SEQ ID NO:135), MNGLRGT (SEQ ID NO:136), RSQEINL (SEQ ID NO:137), YGTVSVP (SEQ ID NO:138), LGSSGVS (SEQ ID NO:139), PGSYVSS (SEQ ID NO:140), ASNLGTY (SEQ ID NO:141), GSEGGSM (SEQ ID NO:142), IASGNSV (SEQ ID NO:143), ATAGAII (SEQ ID NO:144), LSSMGQL (SEQ ID NO:145), and GEKFMGV (SEQ ID NO:146).

Embodiment 10: The AAV vector of embodiment 5, wherein said vector transduces skeletal muscle and said peptide insert comprises or consists of one or more amino acid sequences selected from the group consisting of GTPSHYG (SEQ ID NO:147), ILSKGAL (SEQ ID NO:148), GMGTVAR (SEQ ID NO:149), RVGHFSA (SEQ ID NO:150), PSMGFTP (SEQ ID NO:151), ALMTGKL (SEQ ID NO:152), PSMTRQT (SEQ ID NO:153), VERGASV (SEQ ID NO:154), KLIGRSP (SEQ ID NO:155), AEAPILL (SEQ ID NO:156), NPNLSLG (SEQ ID NO:157), TMKMFAS (SEQ ID NO:158), SAYNRAA (SEQ ID NO:159), IASTVYG (SEQ ID NO:160), VDSSRVR (SEQ ID NO:161), MNGLRGT (SEQ ID NO:162), YGNSKVV (SEQ ID NO:163), SAVYTRA (SEQ ID NO:164), LAITVAT (SEQ ID NO:165), FVKANVL (SEQ ID NO:166), GEDIGEH (SEQ ID NO:167), KSQLSYV (SEQ ID NO:168), ITLSGSS (SEQ ID NO:169), AKFPVKV (SEQ ID NO:170), SLTAMRG (SEQ ID NO:171), RSEYVRV (SEQ ID NO:172), WNGELVA (SEQ ID NO:173), GQSNISR (SEQ ID NO:174), AEAPILL (SEQ ID NO:175), YGTVSVP (SEQ ID NO:176), GLLNSSP (SEQ ID NO:177), YESSRTA (SEQ ID NO:178), ESGNIRP (SEQ ID NO:179), LNSSSYG (SEQ ID NO:180), DNRTVNV (SEQ ID NO:181), SGSGSFM (SEQ ID NO:182), TRLNTVQ (SEQ ID NO:183), VSGGQTQ (SEQ ID NO:184), DGTYKGA (SEQ ID NO:185), RQTNQIE (SEQ ID NO:186), GTASSRP (SEQ ID NO:187), LGSSGVS (SEQ ID NO:188), AHSGGTY (SEQ ID NO:189), SHEVVRA (SEQ ID NO:190), YISESRP (SEQ ID NO:191), SLGGLGN (SEQ ID NO:192), NSYSVSP (SEQ ID NO:193), GSITYTP (SEQ ID NO:194), NGINGAS (SEQ ID NO:195), VSGSSFG (SEQ ID NO:196), EVRVANA (SEQ ID NO:197), SGRNSDL (SEQ ID NO:198), MTGLRGT (SEQ ID NO:199), GFNHGAN (SEQ ID NO:200), GENAGRA (SEQ ID NO:201), QNMVVTS (SEQ ID NO:202), GASPSSI (SEQ ID NO:203), YVNKSQA (SEQ ID NO:204), GTPSQYG (SEQ ID NO:205), NHSSGAM (SEQ ID NO:206), TNLNLRE (SEQ ID NO:207), and GRISGGS (SEQ ID NO:208).

Embodiment 11: The AAV vector of embodiment 10, wherein said vector transduces skeletal muscle and said peptide insert comprises or consists of one or more amino acid sequences selected from the group consisting of GTPSHYG (SEQ ID NO:147), ILSKGAL (SEQ ID NO:148), GMGTVAR (SEQ ID NO:149), RVGHFSA (SEQ ID NO:150), PSMGFTP (SEQ ID NO:151), ALMTGKL (SEQ ID NO:152), PSMTRQT (SEQ ID NO:153), VERGASV (SEQ ID NO:154), KLIGRSP (SEQ ID NO:155), AEAPILL (SEQ ID NO:156), NPNLSLG (SEQ ID NO:157), TMKMFAS (SEQ ID NO:158), SAYNRAA (SEQ ID NO:159), IASTVYG (SEQ ID NO:160), VDSSRVR (SEQ ID NO:161), MNGLRGT (SEQ ID NO:162), YGNSKVV (SEQ ID NO:163), SAVYTRA (SEQ ID NO:164), LAITVAT (SEQ ID NO:165), FVKANVL (SEQ ID NO:166), GEDIGEH (SEQ ID NO:167), KSQLSYV (SEQ ID NO:168), ITLSGSS (SEQ ID NO:169), AKFPVKV (SEQ ID NO:170), SLTAMRG (SEQ ID NO:171), and RSEYVRV (SEQ ID NO:172).

Embodiment 12: The AAV vector of embodiment 10, wherein said vector transduces skeletal muscle and said peptide insert comprises or consists of one or more amino acid sequences selected from the group consisting of WNGELVA (SEQ ID NO:173), GQSNISR (SEQ ID NO:174), AEAPILL (SEQ ID NO:175), YGTVSVP (SEQ ID NO:176), GLLNSSP (SEQ ID NO:177), YESSRTA (SEQ ID NO:178), ESGNIRP (SEQ ID NO:179), LNSSSYG (SEQ ID NO:180), DNRTVNV (SEQ ID NO:181), SGSGSFM (SEQ ID NO:182), TRLNTVQ (SEQ ID NO:183), VSGGQTQ (SEQ ID NO:184), DGTYKGA (SEQ ID NO:185), RQTNQIE (SEQ ID NO:186), GTASSRP (SEQ ID NO:187), LGSSGVS (SEQ ID NO:188), AHSGGTY (SEQ ID NO:189), SHEVVRA (SEQ ID NO:190), YISESRP (SEQ ID NO:191), SLGGLGN (SEQ ID NO:192), NSYSVSP (SEQ ID NO:193), GSITYTP (SEQ ID NO:194), NGINGAS (SEQ ID NO:195), VSGSSFG (SEQ ID NO:196), EVRVANA (SEQ ID NO:197), SGRNSDL (SEQ ID NO:198), MTGLRGT (SEQ ID NO:199), GFNHGAN (SEQ ID NO:200), GENAGRA (SEQ ID NO:201), QNMVVTS (SEQ ID NO:202), GASPSSI (SEQ ID NO:203), YVNKSQA (SEQ ID NO:204), GTPSQYG (SEQ ID NO:205), NHSSGAM (SEQ ID NO:206), TNLNLRE (SEQ ID NO:207), and GRISGGS (SEQ ID NO:208).

Embodiment 13: The AAV vector of embodiment 5, wherein said vector transduces muscle stem cells and said peptide insert comprises or consists of an amino acid sequence selected from the group consisting of VRDLTRV (SEQ ID NO:209), GIGHLAS (SEQ ID NO:210), HESRPPY (SEQ ID NO:211), SSTQKVS (SEQ ID NO:212), FGVSRST (SEQ ID NO:213), MIRGIPS (SEQ ID NO:214), IRQPTSH (SEQ ID NO:215), VSGAVLG (SEQ ID NO:216), RNSDNLS (SEQ ID NO:217), RLASFNS (SEQ ID NO:218), YRIGEGT (SEQ ID NO:219), VHSLGHM (SEQ ID NO:220), VTAVAKV (SEQ ID NO:221), LHHSPSY (SEQ ID NO:222), ERARSTN (SEQ ID NO:223), GMGLTVA (SEQ ID NO:224), LNSSSYG (SEQ ID NO:225), GYKQVSN (SEQ ID NO:226), GKASKDT (SEQ ID NO:227), QLSSRGA (SEQ ID NO:228), VGTRFTS (SEQ ID NO:229), QRSSNGP (SEQ ID NO:230), YKTQGLA (SEQ ID NO:231), RDSMVNP (SEQ ID NO:232), RVSSNLS (SEQ ID NO:233), DGTYKGA (SEQ ID NO:234), FGSGALA (SEQ ID NO:235), ARSQEVP (SEQ ID NO:236), VVVVGRP (SEQ ID NO:237), LHGARSS (SEQ ID NO:238), SGQMRGA (SEQ ID NO:239), TRLNTVQ (SEQ ID NO:240), HMHVCVP (SEQ ID NO:241), VTNSSRV (SEQ ID NO:242), NQLPHFK (SEQ ID NO:243), VRFGSNP (SEQ ID NO:244), YISESRP (SEQ ID NO:245), VSRSGGV (SEQ ID NO:246), LSSNANL (SEQ ID NO:247), DNRTVNV (SEQ ID NO:248), LRVGNVE (SEQ ID NO:249), GTASSRP (SEQ ID NO:250), LHGARYS (SEQ ID NO:251), TGVNVKS (SEQ ID NO:252), VTSWTKN (SEQ ID NO:253), RGSTMMP (SEQ ID NO:254), SGSGSFM (SEQ ID NO:255), VSGGQTQ (SEQ ID NO:256), GMGTVAR (SEQ ID NO:257), LNPRSSS (SEQ ID NO:258), SQNLLSN (SEQ ID NO:259), YRIGEGT (SEQ ID NO:260), STVNGNQ (SEQ ID NO:261), AADAVRV (SEQ ID NO:262), YGNSKVV (SEQ ID NO:263), VPGEGRV (SEQ ID NO:264), GGDAARY (SEQ ID NO:265), YDQVSHP (SEQ ID NO:266), GSFHGTV (SEQ ID NO:277), GAHPTGG (SEQ ID NO:278), RDGQSRG (SEQ ID NO:279), SLGGLGN (SEQ ID NO:280), DGTYKGA (SEQ ID NO:281), GIGHLAS (SEQ ID NO:282), IGSVGKL (SEQ ID NO:283), SHEVVRA (SEQ ID NO:284), EVGHIRG (SEQ ID NO:285), and GLRGVSV (SEQ ID NO:286).

Embodiment 14: The AAV vector of embodiment 13, wherein said vector transduces muscle stem cells and said peptide insert comprises or consists of one or more an amino acid sequences selected from the group consisting of VRDLTRV (SEQ ID NO:209), GIGHLAS (SEQ ID NO:210), HESRPPY (SEQ ID NO:211), SSTQKVS (SEQ ID NO:212), FGVSRST (SEQ ID NO:213), MIRGIPS (SEQ ID NO:214), IRQPTSH (SEQ ID NO:215), VSGAVLG (SEQ ID NO:216), RNSDNLS (SEQ ID NO:217), RLASFNS (SEQ ID NO:218), YRIGEGT (SEQ ID NO:219), VHSLGHM (SEQ ID NO:220), VTAVAKV (SEQ ID NO:221), LHHSPSY (SEQ ID NO:222), ERARSTN (SEQ ID NO:223), GMGLTVA (SEQ ID NO:224), LNSSSYG (SEQ ID NO:225), GYKQVSN (SEQ ID NO:226), GKASKDT (SEQ ID NO:227), QLSSRGA (SEQ ID NO:228), VGTRFTS (SEQ ID NO:229), QRSSNGP (SEQ ID NO:230), YKTQGLA (SEQ ID NO:231), RDSMVNP (SEQ ID NO:232), RVSSNLS (SEQ ID NO:233), and DGTYKGA (SEQ ID NO:234).

Embodiment 15: The AAV vector of embodiment 13, wherein said vector transduces muscle stem cells and said peptide insert comprises or consists of one or more an amino acid sequences selected from the group consisting of FGSGALA (SEQ ID NO:235), ARSQEVP (SEQ ID NO:236), VVVVGRP (SEQ ID NO:237), LHGARSS (SEQ ID NO:238), SGQMRGA (SEQ ID NO:239), TRLNTVQ (SEQ ID NO:240), HMHVCVP (SEQ ID NO:241), VTNSSRV (SEQ ID NO:242), NQLPHFK (SEQ ID NO:243), VRFGSNP (SEQ ID NO:244), YISESRP (SEQ ID NO:245), VSRSGGV (SEQ ID NO:246), LSSNANL (SEQ ID NO:247), DNRTVNV (SEQ ID NO:248), LRVGNVE (SEQ ID NO:249), GTASSRP (SEQ ID NO:250), LHGARYS (SEQ ID NO:251), TGVNVKS (SEQ ID NO:252), VTSWTKN (SEQ ID NO:253), RGSTMMP (SEQ ID NO:254), SGSGSFM (SEQ ID NO:255), VSGGQTQ (SEQ ID NO:256), GMGTVAR (SEQ ID NO:257), LNPRSSS (SEQ ID NO:258), SQNLLSN (SEQ ID NO:259), YRIGEGT (SEQ ID NO:260), STVNGNQ (SEQ ID NO:261), AADAVRV (SEQ ID NO:262), YGNSKVV (SEQ ID NO:263), VPGEGRV (SEQ ID NO:264), GGDAARY (SEQ ID NO:265), YDQVSHP (SEQ ID NO:266), GSFHGTV (SEQ ID NO:287), GAHPTGG (SEQ ID NO:288), RDGQSRG (SEQ ID NO:289), SLGGLGN (SEQ ID NO:290), DGTYKGA (SEQ ID NO:291), GIGHLAS (SEQ ID NO:292), IGSVGKL (SEQ ID NO:293), SHEVVRA (SEQ ID NO:294), EVGHIRG (SEQ ID NO:295), GLRGVSV (SEQ ID NO:296).

Embodiment 16: The AAV vector of embodiment 5, wherein said peptide insert comprises or consists of the amino acid sequence GGMRRDP (SEQ ID NO:1).

Embodiment 17: The AAV vector of embodiment 16, wherein said peptide insert comprises or consists of the amino acid sequence GVAISGF (SEQ ID NO:2).

Embodiment 18: The AAV vector according to any one of embodiments 16-17, wherein said peptide insert comprises or consists of the amino acid sequence GQKWNVD (SEQ ID NO:3).

Embodiment 19: The AAV vector according to any one of embodiments 16-18, wherein said peptide insert comprises or consists of the amino acid sequence GKVGVWA (SEQ ID NO:4).

Embodiment 20: The AAV vector according to any one of embodiments 16-19, wherein said peptide insert comprises or consists of the amino acid sequence GAFELHN (SEQ ID NO:5).

Embodiment 21: The AAV vector according to any one of embodiments 16-20, wherein said peptide insert comprises or consists of the amino acid sequence QVLRPTY (SEQ ID NO:6).

Embodiment 22: The AAV vector according to any one of embodiments 16-21, wherein said peptide insert comprises or consists of the amino acid sequence LGRVHSL (SEQ ID NO:7).

Embodiment 23: The AAV vector according to any one of embodiments 16-22, wherein said peptide insert comprises or consists of the amino acid sequence LYANVSP (SEQ ID NO:8).

Embodiment 24: The AAV vector according to any one of embodiments 16-23, wherein said peptide insert comprises or consists of the amino acid sequence LSLGPTV (SEQ ID NO:9).

Embodiment 25: The AAV vector according to any one of embodiments 16-24, wherein said peptide insert comprises or consists of the amino acid sequence LSLSLAT (SEQ ID NO:10).

Embodiment 26: The AAV vector according to any one of embodiments 16-25, wherein said peptide insert comprises or consists of the amino acid sequence LNSSSYG (SEQ ID NO:11).

Embodiment 27: The AAV vector according to any one of embodiments 16-26, wherein said peptide insert comprises or consists of the amino acid sequence LLRNSTA (SEQ ID NO:12).

Embodiment 28: The AAV vector according to any one of embodiments 16-27, wherein said peptide insert comprises or consists of the amino acid sequence FHSSASA (SEQ ID NO:13).

Embodiment 29: The AAV vector according to any one of embodiments 16-28, wherein said peptide insert comprises or consists of the amino acid sequence FVGDTVA (SEQ ID NO:14).

Embodiment 30: The AAV vector according to any one of embodiments 16-29, wherein said peptide insert comprises or consists of the amino acid sequence FGRGSSG (SEQ ID NO:15).

Embodiment 31: The AAV vector according to any one of embodiments 16-30, wherein said peptide insert comprises or consists of the amino acid sequence WDKAATF (SEQ ID NO:16).

Embodiment 32: The AAV vector according to any one of embodiments 16-31, wherein said peptide insert comprises or consists of the amino acid sequence AEAPILL (SEQ ID NO:17).

Embodiment 33: The AAV vector according to any one of embodiments 16-32, wherein said peptide insert comprises or consists of the amino acid sequence AFHGLLA (SEQ ID NO:18).

Embodiment 34: The AAV vector according to any one of embodiments 16-33, wherein said peptide insert comprises or consists of the amino acid sequence AGYSIVH (SEQ ID NO:19).

Embodiment 35: The AAV vector according to any one of embodiments 16-34, wherein said peptide insert comprises or consists of the amino acid sequence ATWNKSQ (SEQ ID NO:20).

Embodiment 36: The AAV vector according to any one of embodiments 16-35, wherein said peptide insert comprises or consists of the amino acid sequence SVHSDYR (SEQ ID NO:21).

Embodiment 37: The AAV vector according to any one of embodiments 16-36, wherein said peptide insert comprises or consists of the amino acid sequence SGRNSDL (SEQ ID NO:22).

Embodiment 38: The AAV vector according to any one of embodiments 16-37, wherein said peptide insert comprises or consists of the amino acid sequence SVFVPMR (SEQ ID NO:23).

Embodiment 39: The AAV vector according to any one of embodiments 16-38, wherein said peptide insert comprises or consists of the amino acid sequence VHGQYVE (SEQ ID NO:24).

Embodiment 40: The AAV vector according to any one of embodiments 16-39, wherein said peptide insert comprises or consists of the amino acid sequence VERGASV (SEQ ID NO:25).

Embodiment 41: The AAV vector according to any one of embodiments 16-40, wherein said peptide insert comprises or consists of the amino acid sequence VSEMKER (SEQ ID NO:26).

Embodiment 42: The AAV vector according to any one of embodiments 16-41, wherein said peptide insert comprises or consists of the amino acid sequence INHNSHI (SEQ ID NO:27).

Embodiment 43: The AAV vector according to any one of embodiments 16-42, wherein said peptide insert comprises or consists of the amino acid sequence GQSNISR (SEQ ID NO:28).

Embodiment 44: The AAV vector according to any one of embodiments 16-43, wherein said peptide insert comprises or consists of the amino acid sequence RQTNQIE (SEQ ID NO:29).

Embodiment 45: The AAV vector according to any one of embodiments 16-44, wherein said peptide insert comprises or consists of the amino acid sequence GPVVQGA (SEQ ID NO:30).

Embodiment 46: The AAV vector according to any one of embodiments 16-45, wherein said peptide insert comprises or consists of the amino acid sequence VSGSSFG (SEQ ID NO:31).

Embodiment 47: The AAV vector according to any one of embodiments 16-46, wherein said peptide insert comprises or consists of the amino acid sequence DVSLTRM (SEQ ID NO:32).

Embodiment 48: The AAV vector according to any one of embodiments 16-47, wherein said peptide insert comprises or consists of the amino acid sequence NGQGILK (SEQ ID NO:33).

Embodiment 49: The AAV vector according to any one of embodiments 16-48, wherein said peptide insert comprises or consists of the amino acid sequence SGDAYRA (SEQ ID NO:34).

Embodiment 50: The AAV vector according to any one of embodiments 16-49, wherein said peptide insert comprises or consists of the amino acid sequence FNNGIHS (SEQ ID NO:35).

Embodiment 51: The AAV vector according to any one of embodiments 16-50, wherein said peptide insert comprises or consists of the amino acid sequence SSYSVSL (SEQ ID NO:36).

Embodiment 52: The AAV vector according to any one of embodiments 16-51, wherein said peptide insert comprises or consists of the amino acid sequence ESGNIRP (SEQ ID NO:37).

Embodiment 53: The AAV vector according to any one of embodiments 16-52, wherein said peptide insert comprises or consists of the amino acid sequence VSGGQTQ (SEQ ID NO:38).

Embodiment 54: The AAV vector according to any one of embodiments 16-53, wherein said peptide insert comprises or consists of the amino acid sequence IVGSGSA (SEQ ID NO:39).

Embodiment 55: The AAV vector according to any one of embodiments 16-54, wherein said peptide insert comprises or consists of the amino acid sequence VSSQGTS (SEQ ID NO:40).

Embodiment 56: The AAV vector according to any one of embodiments 16-55, wherein said peptide insert comprises or consists of the amino acid sequence GSTYENS (SEQ ID NO:41).

Embodiment 57: The AAV vector according to any one of embodiments 16-56, wherein said peptide insert comprises or consists of the amino acid sequence NGSTSPV (SEQ ID NO:42).

Embodiment 58: The AAV vector according to any one of embodiments 16-57, wherein said peptide insert comprises or consists of the amino acid sequence EGKSSPT (SEQ ID NO:43).

Embodiment 59: The AAV vector according to any one of embodiments 16-58, wherein said peptide insert comprises or consists of the amino acid sequence VGQGISL (SEQ ID NO:44).

Embodiment 60: The AAV vector according to any one of embodiments 16-59, wherein said peptide insert comprises or consists of the amino acid sequence AGIGQAG (SEQ ID NO:45).

Embodiment 61: The AAV vector according to any one of embodiments 16-60, wherein said peptide insert comprises or consists of the amino acid sequence ITSPSVS (SEQ ID NO:46).

Embodiment 62: The AAV vector according to any one of embodiments 16-61, wherein said peptide insert comprises or consists of the amino acid sequence EHVEGRR (SEQ ID NO:47).

Embodiment 63: The AAV vector according to any one of embodiments 16-62, wherein said peptide insert comprises or consists of the amino acid sequence LDGVSHM (SEQ ID NO:48).

Embodiment 64: The AAV vector according to any one of embodiments 16-63, wherein said peptide insert comprises or consists of the amino acid sequence GGGTLHH (SEQ ID NO:49).

Embodiment 65: The AAV vector according to any one of embodiments 16-64, wherein said peptide insert comprises or consists of the amino acid sequence HESRPPY (SEQ ID NO:50).

Embodiment 66: The AAV vector according to any one of embodiments 16-65, wherein said peptide insert comprises or consists of the amino acid sequence SNHDSRV (SEQ ID NO:51).

Embodiment 67: The AAV vector according to any one of embodiments 16-66, wherein said peptide insert comprises or consists of the amino acid sequence SGSGSFM (SEQ ID NO:52).

Embodiment 68: The AAV vector according to any one of embodiments 16-67, wherein said peptide insert comprises or consists of the amino acid sequence VGSLYQP (SEQ ID NO:53).

Embodiment 69: The AAV vector according to any one of embodiments 16-68, wherein said peptide insert comprises or consists of the amino acid sequence GQDNRAL (SEQ ID NO:54).

Embodiment 70: The AAV vector according to any one of embodiments 16-69, wherein said peptide insert comprises or consists of the amino acid sequence RSEYNSV (SEQ ID NO:55).

Embodiment 71: The AAV vector according to any one of embodiments 16-70, wherein said peptide insert comprises or consists of the amino acid sequence TGQTVLG (SEQ ID NO:56).

Embodiment 72: The AAV vector according to any one of embodiments 16-71, wherein said peptide insert comprises or consists of the amino acid sequence GGDAARY (SEQ ID NO:57).

Embodiment 73: The AAV vector according to any one of embodiments 16-72, wherein said peptide insert comprises or consists of the amino acid sequence RGSNDLP (SEQ ID NO:58).

Embodiment 74: The AAV vector according to any one of embodiments 16-73, wherein said peptide insert comprises or consists of the amino acid sequence VASGTVL (SEQ ID NO:59).

Embodiment 75: The AAV vector according to any one of embodiments 16-74, wherein said peptide insert comprises or consists of the amino acid sequence GDHNTIK (SEQ ID NO:60).

Embodiment 76: The AAV vector according to any one of embodiments 16-75, wherein said peptide insert comprises or consists of the amino acid sequence NARDSGF (SEQ ID NO:61).

Embodiment 77: The AAV vector according to any one of embodiments 16-76, wherein said peptide insert comprises or consists of the amino acid sequence PGNINNN (SEQ ID NO:62).

Embodiment 78: The AAV vector according to any one of embodiments 16-77, wherein said peptide insert comprises or consists of the amino acid sequence SCGDSCR (SEQ ID NO:63).

Embodiment 79: The AAV vector according to any one of embodiments 16-78, wherein said peptide insert comprises or consists of the amino acid sequence FGSVHSP (SEQ ID NO:64).

Embodiment 80: The AAV vector according to any one of embodiments 16-79, wherein said peptide insert comprises or consists of the amino acid sequence SLGGLGN (SEQ ID NO:65).

Embodiment 81: The AAV vector according to any one of embodiments 16-80, wherein said peptide insert comprises or consists of the amino acid sequence AADAVRV (SEQ ID NO:66).

Embodiment 82: The AAV vector according to any one of embodiments 16-81, wherein said peptide insert comprises or consists of the amino acid sequence VIAQGMS (SEQ ID NO:67).

Embodiment 83: The AAV vector according to any one of embodiments 16-82, wherein said peptide insert comprises or consists of the amino acid sequence NHSSGAM (SEQ ID NO:68).

Embodiment 84: The AAV vector according to any one of embodiments 16-83, wherein said peptide insert comprises or consists of the amino acid sequence LRDNSAF (SEQ ID NO:69).

Embodiment 85: The AAV vector according to any one of embodiments 16-84, wherein said peptide insert comprises or consists of the amino acid sequence LGSGALA (SEQ ID NO:70).

Embodiment 86: The AAV vector according to any one of embodiments 16-85, wherein said peptide insert comprises or consists of the amino acid sequence SHEVVRA (SEQ ID NO:71).

Embodiment 87: The AAV vector according to any one of embodiments 16-86, wherein said peptide insert comprises or consists of the amino acid sequence FNISANE (SEQ ID NO:72).

Embodiment 88: The AAV vector according to any one of embodiments 16-87, wherein said peptide insert comprises or consists of the amino acid sequence ARSQEVP (SEQ ID NO:73).

Embodiment 89: The AAV vector according to any one of embodiments 16-88, wherein said peptide insert comprises or consists of the amino acid sequence AGNITSF (SEQ ID NO:74).

Embodiment 90: The AAV vector according to any one of embodiments 16-89, wherein said peptide insert comprises or consists of the amino acid sequence TCNSVGC (SEQ ID NO:75).

Embodiment 91: The AAV vector according to any one of embodiments 16-90, wherein said peptide insert comprises or consists of the amino acid sequence GGGSSSA (SEQ ID NO:76).

Embodiment 92: The AAV vector according to any one of embodiments 16-91, wherein said peptide insert comprises or consists of the amino acid sequence YDQVSHP (SEQ ID NO:77).

Embodiment 93: The AAV vector according to any one of embodiments 16-92, wherein said peptide insert comprises or consists of the amino acid sequence VHGISPL (SEQ ID NO:78).

Embodiment 94: The AAV vector according to any one of embodiments 16-93, wherein said peptide insert comprises or consists of the amino acid sequence TGLNYTT (SEQ ID NO:79).

Embodiment 95: The AAV vector according to any one of embodiments 16-94, wherein said peptide insert comprises or consists of the amino acid sequence QDRNYGV (SEQ ID NO:80).

Embodiment 96: The AAV vector according to any one of embodiments 16-95, wherein said peptide insert comprises or consists of the amino acid sequence VSEINRG (SEQ ID NO:81).

Embodiment 97: The AAV vector according to any one of embodiments 16-96, wherein said peptide insert comprises or consists of the amino acid sequence QRSSNGP (SEQ ID NO:82).

Embodiment 98: The AAV vector according to any one of embodiments 16-97, wherein said peptide insert comprises or consists of the amino acid sequence SSTGTWV (SEQ ID NO:83).

Embodiment 99: The AAV vector according to any one of embodiments 16-98, wherein said peptide insert comprises or consists of the amino acid sequence QLPNNLS (SEQ ID NO:84).

Embodiment 100: The AAV vector according to any one of embodiments 16-99, wherein said peptide insert comprises or consists of the amino acid sequence YNGAIHH (SEQ ID NO:85).

Embodiment 101: The AAV vector according to any one of embodiments 16-100, wherein said peptide insert comprises or consists of the amino acid sequence MRNTESS (SEQ ID NO:86).

Embodiment 102: The AAV vector according to any one of embodiments 16-101, wherein said peptide insert comprises or consists of the amino acid sequence GASPSSI (SEQ ID NO:87).

Embodiment 103: The AAV vector according to any one of embodiments 16-102, wherein said peptide insert comprises or consists of the amino acid sequence DGTYKGA (SEQ ID NO:88).

Embodiment 104: The AAV vector according to any one of embodiments 16-103, wherein said peptide insert comprises or consists of the amino acid sequence GQGCTQC (SEQ ID NO:89).

Embodiment 105: The AAV vector according to any one of embodiments 16-104, wherein said peptide insert comprises or consists of the amino acid sequence GAHPTGG (SEQ ID NO:90).

Embodiment 106: The AAV vector according to any one of embodiments 16-105, wherein said peptide insert comprises or consists of the amino acid sequence SNYNVTG (SEQ ID NO:91).

Embodiment 107: The AAV vector according to any one of embodiments 16-106, wherein said peptide insert comprises or consists of the amino acid sequence SANVFSP (SEQ ID NO:92).

Embodiment 108: The AAV vector according to any one of embodiments 16-107, wherein said peptide insert comprises or consists of the amino acid sequence AHSGGTY (SEQ ID NO:93).

Embodiment 109: The AAV vector according to any one of embodiments 16-108, wherein said peptide insert comprises or consists of the amino acid sequence GENAGRA (SEQ ID NO:94).

Embodiment 110: The AAV vector according to any one of embodiments 16-109, wherein said peptide insert comprises or consists of the amino acid sequence IGCDYPC (SEQ ID NO:95).

Embodiment 111: The AAV vector according to any one of embodiments 16-110, wherein said peptide insert comprises or consists of the amino acid sequence ISLPNSI (SEQ ID NO:96).

Embodiment 112: The AAV vector according to any one of embodiments 16-111, wherein said peptide insert comprises or consists of the amino acid sequence NGINGAS (SEQ ID NO:97).

Embodiment 113: The AAV vector according to any one of embodiments 16-112, wherein said peptide insert comprises or consists of the amino acid sequence GPSSLRE (SEQ ID NO:98).

Embodiment 114: The AAV vector according to any one of embodiments 16-113, wherein said peptide insert comprises or consists of the amino acid sequence VQSGNYV (SEQ ID NO:99).

Embodiment 115: The AAV vector according to any one of embodiments 16-114, wherein said peptide insert comprises or consists of the amino acid sequence GGYSETS (SEQ ID NO:100).

Embodiment 116: The AAV vector according to any one of embodiments 16-115, wherein said peptide insert comprises or consists of the amino acid sequence LRVGNVE (SEQ ID NO:101).

Embodiment 117: The AAV vector according to any one of embodiments 16-116, wherein said peptide insert comprises or consists of the amino acid sequence GMGTVAR (SEQ ID NO:102).

Embodiment 118: The AAV vector according to any one of embodiments 16-117, wherein said peptide insert comprises or consists of the amino acid sequence VQMSGYL (SEQ ID NO:103).

Embodiment 119: The AAV vector according to any one of embodiments 16-118, wherein said peptide insert comprises or consists of the amino acid sequence QNMVVTS (SEQ ID NO:104).

Embodiment 120: The AAV vector according to any one of embodiments 16-119, wherein said peptide insert comprises or consists of the amino acid sequence THGLPIV (SEQ ID NO:105).

Embodiment 121: The AAV vector according to any one of embodiments 16-120, wherein said peptide insert comprises or consists of the amino acid sequence GFNHGAN (SEQ ID NO:106).

Embodiment 122: The AAV vector according to any one of embodiments 16-121, wherein said peptide insert comprises or consists of the amino acid sequence NSYSVSP (SEQ ID NO:107).

Embodiment 123: The AAV vector according to any one of embodiments 16-122, wherein said peptide insert comprises or consists of the amino acid sequence SMLGGVA (SEQ ID NO:108).

Embodiment 124: The AAV vector according to any one of embodiments 16-123, wherein said peptide insert comprises or consists of the amino acid sequence YGVDMGL (SEQ ID NO:109).

Embodiment 125: The AAV vector according to any one of embodiments 16-124, wherein said peptide insert comprises or consists of the amino acid sequence VIGHGSA (SEQ ID NO:110).

Embodiment 126: The AAV vector according to any one of embodiments 16-125, wherein said peptide insert comprises or consists of the amino acid sequence SGLDKAP (SEQ ID NO:111).

Embodiment 127: The AAV vector according to any one of embodiments 16-126, wherein said peptide insert comprises or consists of the amino acid sequence DVKNGGR (SEQ ID NO:112).

Embodiment 128: The AAV vector according to any one of embodiments 16-127, wherein said peptide insert comprises or consists of the amino acid sequence IGNGGLI (SEQ ID NO: 113).

Embodiment 129: The AAV vector according to any one of embodiments 16-128, wherein said peptide insert comprises or consists of the amino acid sequence QVGFPTP (SEQ ID NO:114).

Embodiment 130: The AAV vector according to any one of embodiments 16-129, wherein said peptide insert comprises or consists of the amino acid sequence LSSNANL (SEQ ID NO: 115).

Embodiment 131: The AAV vector according to any one of embodiments 16-130, wherein said peptide insert comprises or consists of the amino acid sequence TNLHVNL (SEQ ID NO:116).

Embodiment 132: The AAV vector according to any one of embodiments 16-131, wherein said peptide insert comprises or consists of the amino acid sequence SYIGVAA (SEQ ID NO: 117).

Embodiment 133: The AAV vector according to any one of embodiments 16-132, wherein said peptide insert comprises or consists of the amino acid sequence STVNGNQ (SEQ ID NO:118).

Embodiment 134: The AAV vector according to any one of embodiments 16-133, wherein said peptide insert comprises or consists of the amino acid sequence TNRDAQF (SEQ ID NO:119).

Embodiment 135: The AAV vector according to any one of embodiments 16-134, wherein said peptide insert comprises or consists of the amino acid sequence LHHSPSY (SEQ ID NO:120).

Embodiment 136: The AAV vector according to any one of embodiments 16-135, wherein said peptide insert comprises or consists of the amino acid sequence TTYNSSA (SEQ ID NO:121).

Embodiment 137: The AAV vector according to any one of embodiments 16-136, wherein said peptide insert comprises or consists of the amino acid sequence PSSQPLP (SEQ ID NO:122).

Embodiment 138: The AAV vector according to any one of embodiments 16-137, wherein said peptide insert comprises or consists of the amino acid sequence RDSMVNP (SEQ ID NO:123).

Embodiment 139: The AAV vector according to any one of embodiments 16-138, wherein said peptide insert comprises or consists of the amino acid sequence SLHSPQL (SEQ ID NO:124).

Embodiment 140: The AAV vector according to any one of embodiments 16-139, wherein said peptide insert comprises or consists of the amino acid sequence VQVGTGT (SEQ ID NO:125).

Embodiment 141: The AAV vector according to any one of embodiments 16-140, wherein said peptide insert comprises or consists of the amino acid sequence RENPSGM (SEQ ID NO:126).

Embodiment 142: The AAV vector according to any one of embodiments 16-141, wherein said peptide insert comprises or consists of the amino acid sequence GVPKTHD (SEQ ID NO:127).

Embodiment 143: The AAV vector according to any one of embodiments 16-142, wherein said peptide insert comprises or consists of the amino acid sequence VDQGRLS (SEQ ID NO:128).

Embodiment 144: The AAV vector according to any one of embodiments 16-143, wherein said peptide insert comprises or consists of the amino acid sequence FDKGAVI (SEQ ID NO:129).

Embodiment 145: The AAV vector according to any one of embodiments 16-144, wherein said peptide insert comprises or consists of the amino acid sequence VSRSGGV (SEQ ID NO:130).

Embodiment 146: The AAV vector according to any one of embodiments 16-145, wherein said peptide insert comprises or consists of the amino acid sequence INMQSMA (SEQ ID NO:131).

Embodiment 147: The AAV vector according to any one of embodiments 16-146, wherein said peptide insert comprises or consists of the amino acid sequence GTASSRP (SEQ ID NO:132).

Embodiment 148: The AAV vector according to any one of embodiments 16-147, wherein said peptide insert comprises or consists of the amino acid sequence EVGHIRG (SEQ ID NO:133).

Embodiment 149: The AAV vector according to any one of embodiments 16-148, wherein said peptide insert comprises or consists of the amino acid sequence RDVSSGG (SEQ ID NO:134).

Embodiment 150: The AAV vector according to any one of embodiments 16-149, wherein said peptide insert comprises or consists of the amino acid sequence ITLSGSS (SEQ ID NO:135).

Embodiment 151: The AAV vector according to any one of embodiments 16-150, wherein said peptide insert comprises or consists of the amino acid sequence MNGLRGT (SEQ ID NO:136).

Embodiment 152: The AAV vector according to any one of embodiments 16-151, wherein said peptide insert comprises or consists of the amino acid sequence RSQEINL (SEQ ID NO:137).

Embodiment 153: The AAV vector according to any one of embodiments 16-152, wherein said peptide insert comprises or consists of the amino acid sequence YGTVSVP (SEQ ID NO:138).

Embodiment 154: The AAV vector according to any one of embodiments 16-153, wherein said peptide insert comprises or consists of the amino acid sequence LGSSGVS (SEQ ID NO:139).

Embodiment 155: The AAV vector according to any one of embodiments 16-154, wherein said peptide insert comprises or consists of the amino acid sequence PGSYVSS (SEQ ID NO:140).

Embodiment 156: The AAV vector according to any one of embodiments 16-155, wherein said peptide insert comprises or consists of the amino acid sequence ASNLGTY (SEQ ID NO:141).

Embodiment 157: The AAV vector according to any one of embodiments 16-156, wherein said peptide insert comprises or consists of the amino acid sequence GSEGGSM (SEQ ID NO:142).

Embodiment 158: The AAV vector according to any one of embodiments 16-157, wherein said peptide insert comprises or consists of the amino acid sequence IASGNSV (SEQ ID NO:143).

Embodiment 159: The AAV vector according to any one of embodiments 16-158, wherein said peptide insert comprises or consists of the amino acid sequence ATAGAII (SEQ ID NO:144).

Embodiment 160: The AAV vector according to any one of embodiments 16-159, wherein said peptide insert comprises or consists of the amino acid sequence LSSMGQL (SEQ ID NO:145).

Embodiment 161: The AAV vector according to any one of embodiments 16-160, wherein said peptide insert comprises or consists of the amino acid sequence GEKFMGV (SEQ ID NO:146).

Embodiment 162: The AAV vector according to any one of embodiments 16-161, wherein said peptide insert comprises or consists of the amino acid sequence GTPSHYG (SEQ ID NO:147).

Embodiment 163: The AAV vector according to any one of embodiments 16-162, wherein said peptide insert comprises or consists of the amino acid sequence ILSKGAL (SEQ ID NO:148).

Embodiment 164: The AAV vector according to any one of embodiments 16-163, wherein said peptide insert comprises or consists of the amino acid sequence GMGTVAR (SEQ ID NO:149).

Embodiment 165: The AAV vector according to any one of embodiments 16-164, wherein said peptide insert comprises or consists of the amino acid sequence RVGHFSA (SEQ ID NO:150).

Embodiment 166: The AAV vector according to any one of embodiments 16-165, wherein said peptide insert comprises or consists of the amino acid sequence PSMGFTP (SEQ ID NO:151).

Embodiment 167: The AAV vector according to any one of embodiments 16-166, wherein said peptide insert comprises or consists of the amino acid sequence ALMTGKL (SEQ ID NO:152).

Embodiment 168: The AAV vector according to any one of embodiments 16-167, wherein said peptide insert comprises or consists of the amino acid sequence PSMTRQT (SEQ ID NO:153).

Embodiment 169: The AAV vector according to any one of embodiments 16-168, wherein said peptide insert comprises or consists of the amino acid sequence VERGASV (SEQ ID NO:154).

Embodiment 170: The AAV vector according to any one of embodiments 16-169, wherein said peptide insert comprises or consists of the amino acid sequence KLIGRSP (SEQ ID NO:155).

Embodiment 171: The AAV vector according to any one of embodiments 16-170, wherein said peptide insert comprises or consists of the amino acid sequence AEAPILL (SEQ ID NO:156).

Embodiment 172: The AAV vector according to any one of embodiments 16-171, wherein said peptide insert comprises or consists of the amino acid sequence NPNLSLG (SEQ ID NO:157).

Embodiment 173: The AAV vector according to any one of embodiments 16-172, wherein said peptide insert comprises or consists of the amino acid sequence TMKMFAS (SEQ ID NO:158).

Embodiment 174: The AAV vector according to any one of embodiments 16-173, wherein said peptide insert comprises or consists of the amino acid sequence SAYNRAA (SEQ ID NO:159).

Embodiment 175: The AAV vector according to any one of embodiments 16-174, wherein said peptide insert comprises or consists of the amino acid sequence IASTVYG (SEQ ID NO:160).

Embodiment 176: The AAV vector according to any one of embodiments 16-175, wherein said peptide insert comprises or consists of the amino acid sequence VDSSRVR (SEQ ID NO:161).

Embodiment 177: The AAV vector according to any one of embodiments 16-176, wherein said peptide insert comprises or consists of the amino acid sequence MNGLRGT (SEQ ID NO:162).

Embodiment 178: The AAV vector according to any one of embodiments 16-177, wherein said peptide insert comprises or consists of the amino acid sequence YGNSKVV (SEQ ID NO:163).

Embodiment 179: The AAV vector according to any one of embodiments 16-178, wherein said peptide insert comprises or consists of the amino acid sequence SAVYTRA (SEQ ID NO:164).

Embodiment 180: The AAV vector according to any one of embodiments 16-179, wherein said peptide insert comprises or consists of the amino acid sequence LAITVAT (SEQ ID NO:165).

Embodiment 181: The AAV vector according to any one of embodiments 16-180, wherein said peptide insert comprises or consists of the amino acid sequence FVKANVL (SEQ ID NO:166).

Embodiment 182: The AAV vector according to any one of embodiments 16-181, wherein said peptide insert comprises or consists of the amino acid sequence GEDIGEH (SEQ ID NO:167).

Embodiment 183: The AAV vector according to any one of embodiments 16-182, wherein said peptide insert comprises or consists of the amino acid sequence KSQLSYV (SEQ ID NO:168).

Embodiment 184: The AAV vector according to any one of embodiments 16-183, wherein said peptide insert comprises or consists of the amino acid sequence ITLSGSS (SEQ ID NO:169).

Embodiment 185: The AAV vector according to any one of embodiments 16-184, wherein said peptide insert comprises or consists of the amino acid sequence AKFPVKV (SEQ ID NO:170).

Embodiment 186: The AAV vector according to any one of embodiments 16-185, wherein said peptide insert comprises or consists of the amino acid sequence SLTAMRG (SEQ ID NO:171).

Embodiment 187: The AAV vector according to any one of embodiments 16-186, wherein said peptide insert comprises or consists of the amino acid sequence RSEYVRV (SEQ ID NO:172).

Embodiment 188: The AAV vector according to any one of embodiments 16-187, wherein said peptide insert comprises or consists of the amino acid sequence WNGELVA (SEQ ID NO:173).

Embodiment 189: The AAV vector according to any one of embodiments 16-188, wherein said peptide insert comprises or consists of the amino acid sequence GQSNISR (SEQ ID NO:174).

Embodiment 190: The AAV vector according to any one of embodiments 16-189, wherein said peptide insert comprises or consists of the amino acid sequence AEAPILL (SEQ ID NO:175).

Embodiment 191: The AAV vector according to any one of embodiments 16-190, wherein said peptide insert comprises or consists of the amino acid sequence YGTVSVP (SEQ ID NO:176).

Embodiment 192: The AAV vector according to any one of embodiments 16-191, wherein said peptide insert comprises or consists of the amino acid sequence GLLNSSP (SEQ ID NO:177).

Embodiment 193: The AAV vector according to any one of embodiments 16-192, wherein said peptide insert comprises or consists of the amino acid sequence YESSRTA (SEQ ID NO:178).

Embodiment 194: The AAV vector according to any one of embodiments 16-193, wherein said peptide insert comprises or consists of the amino acid sequence ESGNIRP (SEQ ID NO:179).

Embodiment 195: The AAV vector according to any one of embodiments 16-194, wherein said peptide insert comprises or consists of the amino acid sequence LNSSSYG (SEQ ID NO:180).

Embodiment 196: The AAV vector according to any one of embodiments 16-195, wherein said peptide insert comprises or consists of the amino acid sequence DNRTVNV (SEQ ID NO:181).

Embodiment 197: The AAV vector according to any one of embodiments 16-196, wherein said peptide insert comprises or consists of the amino acid sequence SGSGSFM (SEQ ID NO:182).

Embodiment 198: The AAV vector according to any one of embodiments 16-197, wherein said peptide insert comprises or consists of the amino acid sequence TRLNTVQ (SEQ ID NO:183).

Embodiment 199: The AAV vector according to any one of embodiments 16-198, wherein said peptide insert comprises or consists of the amino acid sequence VSGGQTQ (SEQ ID NO:184).

Embodiment 200: The AAV vector according to any one of embodiments 16-199, wherein said peptide insert comprises or consists of the amino acid sequence DGTYKGA (SEQ ID NO:185).

Embodiment 201: The AAV vector according to any one of embodiments 16-200, wherein said peptide insert comprises or consists of the amino acid sequence RQTNQIE (SEQ ID NO:186).

Embodiment 202: The AAV vector according to any one of embodiments 16-201, wherein said peptide insert comprises or consists of the amino acid sequence GTASSRP (SEQ ID NO:187).

Embodiment 203: The AAV vector according to any one of embodiments 16-202, wherein said peptide insert comprises or consists of the amino acid sequence LGSSGVS (SEQ ID NO:188).

Embodiment 204: The AAV vector according to any one of embodiments 16-203, wherein said peptide insert comprises or consists of the amino acid sequence AHSGGTY (SEQ ID NO:189).

Embodiment 205: The AAV vector according to any one of embodiments 16-204, wherein said peptide insert comprises or consists of the amino acid sequence SHEVVRA (SEQ ID NO:190).

Embodiment 206: The AAV vector according to any one of embodiments 16-205, wherein said peptide insert comprises or consists of the amino acid sequence YISESRP (SEQ ID NO:191).

Embodiment 207: The AAV vector according to any one of embodiments 16-206, wherein said peptide insert comprises or consists of the amino acid sequence SLGGLGN (SEQ ID NO:192).

Embodiment 208: The AAV vector according to any one of embodiments 16-207, wherein said peptide insert comprises or consists of the amino acid sequence NSYSVSP (SEQ ID NO:193).

Embodiment 209: The AAV vector according to any one of embodiments 16-208, wherein said peptide insert comprises or consists of the amino acid sequence GSITYTP (SEQ ID NO:194).

Embodiment 210: The AAV vector according to any one of embodiments 16-209, wherein said peptide insert comprises or consists of the amino acid sequence NGINGAS (SEQ ID NO:195).

Embodiment 211: The AAV vector according to any one of embodiments 16-210, wherein said peptide insert comprises or consists of the amino acid sequence VSGSSFG (SEQ ID NO:196).

Embodiment 212: The AAV vector according to any one of embodiments 16-211, wherein said peptide insert comprises or consists of the amino acid sequence EVRVANA (SEQ ID NO:197).

Embodiment 213: The AAV vector according to any one of embodiments 16-212, wherein said peptide insert comprises or consists of the amino acid sequence SGRNSDL (SEQ ID NO:198).

Embodiment 214: The AAV vector according to any one of embodiments 16-213, wherein said peptide insert comprises or consists of the amino acid sequence MTGLRGT (SEQ ID NO:199).

Embodiment 215: The AAV vector according to any one of embodiments 16-214, wherein said peptide insert comprises or consists of the amino acid sequence GFNHGAN (SEQ ID NO:200).

Embodiment 216: The AAV vector according to any one of embodiments 16-215, wherein said peptide insert comprises or consists of the amino acid sequence GENAGRA (SEQ ID NO:201).

Embodiment 217: The AAV vector according to any one of embodiments 16-216, wherein said peptide insert comprises or consists of the amino acid sequence QNMVVTS (SEQ ID NO:202).

Embodiment 218: The AAV vector according to any one of embodiments 16-217, wherein said peptide insert comprises or consists of the amino acid sequence GASPSSI (SEQ ID NO:203).

Embodiment 219: The AAV vector according to any one of embodiments 16-218, wherein said peptide insert comprises or consists of the amino acid sequence YVNKSQA (SEQ ID NO:204).

Embodiment 220: The AAV vector according to any one of embodiments 16-219, wherein said peptide insert comprises or consists of the amino acid sequence GTPSQYG (SEQ ID NO:205).

Embodiment 221: The AAV vector according to any one of embodiments 16-220, wherein said peptide insert comprises or consists of the amino acid sequence NHSSGAM (SEQ ID NO:206).

Embodiment 222: The AAV vector according to any one of embodiments 16-221, wherein said peptide insert comprises or consists of the amino acid sequence TNLNLRE (SEQ ID NO:207).

Embodiment 223: The AAV vector according to any one of embodiments 16-222, wherein said peptide insert comprises or consists of the amino acid sequence GRISGGS (SEQ ID NO:208).

Embodiment 224: The AAV vector according to any one of embodiments 16-223, wherein said peptide insert comprises or consists of the amino acid sequence VRDLTRV (SEQ ID NO:209).

Embodiment 225: The AAV vector according to any one of embodiments 16-224, wherein said peptide insert comprises or consists of the amino acid sequence GIGHLAS (SEQ ID NO:210).

Embodiment 226: The AAV vector according to any one of embodiments 16-225, wherein said peptide insert comprises or consists of the amino acid sequence HESRPPY (SEQ ID NO:211).

Embodiment 227: The AAV vector according to any one of embodiments 16-226, wherein said peptide insert comprises or consists of the amino acid sequence SSTQKVS (SEQ ID NO:212).

Embodiment 228: The AAV vector according to any one of embodiments 16-227, wherein said peptide insert comprises or consists of the amino acid sequence FGVSRST (SEQ ID NO:213).

Embodiment 229: The AAV vector according to any one of embodiments 16-228, wherein said peptide insert comprises or consists of the amino acid sequence MIRGIPS (SEQ ID NO:214).

Embodiment 230: The AAV vector according to any one of embodiments 16-229, wherein said peptide insert comprises or consists of the amino acid sequence IRQPTSH (SEQ ID NO:215).

Embodiment 231: The AAV vector according to any one of embodiments 16-230, wherein said peptide insert comprises or consists of the amino acid sequence VSGAVLG (SEQ ID NO:216).

Embodiment 232: The AAV vector according to any one of embodiments 16-231, wherein said peptide insert comprises or consists of the amino acid sequence RNSDNLS (SEQ ID NO:217).

Embodiment 233: The AAV vector according to any one of embodiments 16-232, wherein said peptide insert comprises or consists of the amino acid sequence RLASFNS (SEQ ID NO:218).

Embodiment 234: The AAV vector according to any one of embodiments 16-233, wherein said peptide insert comprises or consists of the amino acid sequence YRIGEGT (SEQ ID NO:219).

Embodiment 235: The AAV vector according to any one of embodiments 16-234, wherein said peptide insert comprises or consists of the amino acid sequence VHSLGHM (SEQ ID NO:220).

Embodiment 236: The AAV vector according to any one of embodiments 16-235, wherein said peptide insert comprises or consists of the amino acid sequence VTAVAKV (SEQ ID NO:221).

Embodiment 237: The AAV vector according to any one of embodiments 16-236, wherein said peptide insert comprises or consists of the amino acid sequence LHHSPSY (SEQ ID NO:222).

Embodiment 238: The AAV vector according to any one of embodiments 16-237, wherein said peptide insert comprises or consists of the amino acid sequence ERARSTN (SEQ ID NO:223).

Embodiment 239: The AAV vector according to any one of embodiments 16-238, wherein said peptide insert comprises or consists of the amino acid sequence GMGLTVA (SEQ ID NO:224).

Embodiment 240: The AAV vector according to any one of embodiments 16-239, wherein said peptide insert comprises or consists of the amino acid sequence LNSSSYG (SEQ ID NO:225).

Embodiment 241: The AAV vector according to any one of embodiments 16-240, wherein said peptide insert comprises or consists of the amino acid sequence GYKQVSN (SEQ ID NO:226).

Embodiment 242: The AAV vector according to any one of embodiments 16-241, wherein said peptide insert comprises or consists of the amino acid sequence GKASKDT (SEQ ID NO:227).

Embodiment 243: The AAV vector according to any one of embodiments 16-242, wherein said peptide insert comprises or consists of the amino acid sequence QLSSRGA (SEQ ID NO:228).

Embodiment 244: The AAV vector according to any one of embodiments 16-243, wherein said peptide insert comprises or consists of the amino acid sequence VGTRFTS (SEQ ID NO:229).

Embodiment 245: The AAV vector according to any one of embodiments 16-244, wherein said peptide insert comprises or consists of the amino acid sequence QRSSNGP (SEQ ID NO:230).

Embodiment 246: The AAV vector according to any one of embodiments 16-245, wherein said peptide insert comprises or consists of the amino acid sequence YKTQGLA (SEQ ID NO:231).

Embodiment 247: The AAV vector according to any one of embodiments 16-246, wherein said peptide insert comprises or consists of the amino acid sequence RDSMVNP (SEQ ID NO:232).

Embodiment 248: The AAV vector according to any one of embodiments 16-247, wherein said peptide insert comprises or consists of the amino acid sequence RVSSNLS (SEQ ID NO:233).

Embodiment 249: The AAV vector according to any one of embodiments 16-248, wherein said peptide insert comprises or consists of the amino acid sequence DGTYKGA (SEQ ID NO:234).

Embodiment 250: The AAV vector according to any one of embodiments 16-249, wherein said peptide insert comprises or consists of the amino acid sequence FGSGALA (SEQ ID NO:235).

Embodiment 251: The AAV vector according to any one of embodiments 16-250, wherein said peptide insert comprises or consists of the amino acid sequence ARSQEVP (SEQ ID NO:236).

Embodiment 252: The AAV vector according to any one of embodiments 16-251, wherein said peptide insert comprises or consists of the amino acid sequence VVVVGRP (SEQ ID NO:237).

Embodiment 253: The AAV vector according to any one of embodiments 16-252, wherein said peptide insert comprises or consists of the amino acid sequence LHGARSS (SEQ ID NO:238).

Embodiment 254: The AAV vector according to any one of embodiments 16-253, wherein said peptide insert comprises or consists of the amino acid sequence SGQMRGA (SEQ ID NO:239).

Embodiment 255: The AAV vector according to any one of embodiments 16-254, wherein said peptide insert comprises or consists of the amino acid sequence TRLNTVQ (SEQ ID NO:240).

Embodiment 256: The AAV vector according to any one of embodiments 16-255, wherein said peptide insert comprises or consists of the amino acid sequence HMHVCVP (SEQ ID NO:241).

Embodiment 257: The AAV vector according to any one of embodiments 16-256, wherein said peptide insert comprises or consists of the amino acid sequence VTNSSRV (SEQ ID NO:242).

Embodiment 258: The AAV vector according to any one of embodiments 16-257, wherein said peptide insert comprises or consists of the amino acid sequence NQLPHFK (SEQ ID NO:243).

Embodiment 259: The AAV vector according to any one of embodiments 16-258, wherein said peptide insert comprises or consists of the amino acid sequence VRFGSNP (SEQ ID NO:244).

Embodiment 260: The AAV vector according to any one of embodiments 16-259, wherein said peptide insert comprises or consists of the amino acid sequence YISESRP (SEQ ID NO:245).

Embodiment 261: The AAV vector according to any one of embodiments 16-260, wherein said peptide insert comprises or consists of the amino acid sequence VSRSGGV (SEQ ID NO:246).

Embodiment 262: The AAV vector according to any one of embodiments 16-261, wherein said peptide insert comprises or consists of the amino acid sequence LSSNANL (SEQ ID NO:247).

Embodiment 263: The AAV vector according to any one of embodiments 16-262, wherein said peptide insert comprises or consists of the amino acid sequence DNRTVNV (SEQ ID NO:248).

Embodiment 264: The AAV vector according to any one of embodiments 16-263, wherein said peptide insert comprises or consists of the amino acid sequence LRVGNVE (SEQ ID NO:249).

Embodiment 265: The AAV vector according to any one of embodiments 16-264, wherein said peptide insert comprises or consists of the amino acid sequence GTASSRP (SEQ ID NO:250).

Embodiment 266: The AAV vector according to any one of embodiments 16-265, wherein said peptide insert comprises or consists of the amino acid sequence LHGARYS (SEQ ID NO:251).

Embodiment 267: The AAV vector according to any one of embodiments 16-266, wherein said peptide insert comprises or consists of the amino acid sequence TGVNVKS (SEQ ID NO:252).

Embodiment 268: The AAV vector according to any one of embodiments 16-267, wherein said peptide insert comprises or consists of the amino acid sequence VTSWTKN (SEQ ID NO:253).

Embodiment 269: The AAV vector according to any one of embodiments 16-268, wherein said peptide insert comprises or consists of the amino acid sequence RGSTMMP (SEQ ID NO:254).

Embodiment 270: The AAV vector according to any one of embodiments 16-269, wherein said peptide insert comprises or consists of the amino acid sequence SGSGSFM (SEQ ID NO:255).

Embodiment 271: The AAV vector according to any one of embodiments 16-270, wherein said peptide insert comprises or consists of the amino acid sequence VSGGQTQ (SEQ ID NO:256).

Embodiment 272: The AAV vector according to any one of embodiments 16-271, wherein said peptide insert comprises or consists of the amino acid sequence GMGTVAR (SEQ ID NO:257).

Embodiment 273: The AAV vector according to any one of embodiments 16-272, wherein said peptide insert comprises or consists of the amino acid sequence LNPRSSS (SEQ ID NO:258).

Embodiment 274: The AAV vector according to any one of embodiments 16-273, wherein said peptide insert comprises or consists of the amino acid sequence SQNLLSN (SEQ ID NO:259).

Embodiment 275: The AAV vector according to any one of embodiments 16-274, wherein said peptide insert comprises or consists of the amino acid sequence YRIGEGT (SEQ ID NO:260).

Embodiment 276: The AAV vector according to any one of embodiments 16-275, wherein said peptide insert comprises or consists of the amino acid sequence STVNGNQ (SEQ ID NO:261).

Embodiment 277: The AAV vector according to any one of embodiments 16-276, wherein said peptide insert comprises or consists of the amino acid sequence AADAVRV (SEQ ID NO:262).

Embodiment 278: The AAV vector according to any one of embodiments 16-277, wherein said peptide insert comprises or consists of the amino acid sequence YGNSKVV (SEQ ID NO:263).

Embodiment 279: The AAV vector according to any one of embodiments 16-278, wherein said peptide insert comprises or consists of the amino acid sequence VPGEGRV (SEQ ID NO:264).

Embodiment 280: The AAV vector according to any one of embodiments 16-279, wherein said peptide insert comprises or consists of the amino acid sequence GGDAARY (SEQ ID NO:265).

Embodiment 281: The AAV vector according to any one of embodiments 16-280, wherein said peptide insert comprises or consists of the amino acid sequence YDQVSHP (SEQ ID NO:266).

Embodiment 282: The AAV vector according to any one of embodiments 16-281, wherein said peptide insert comprises or consists of the amino acid sequence GSFHGTV (SEQ ID NO:267).

Embodiment 283: The AAV vector according to any one of embodiments 16-282, wherein said peptide insert comprises or consists of the amino acid sequence GAHPTGG (SEQ ID NO:268).

Embodiment 284: The AAV vector according to any one of embodiments 16-283, wherein said peptide insert comprises or consists of the amino acid sequence RDGQSRG (SEQ ID NO:269).

Embodiment 285: The AAV vector according to any one of embodiments 16-284, wherein said peptide insert comprises or consists of the amino acid sequence SLGGLGN (SEQ ID NO:270).

Embodiment 286: The AAV vector according to any one of embodiments 16-285, wherein said peptide insert comprises or consists of the amino acid sequence DGTYKGA (SEQ ID NO:271).

Embodiment 287: The AAV vector according to any one of embodiments 16-286, wherein said peptide insert comprises or consists of the amino acid sequence GIGHLAS (SEQ ID NO:272).

Embodiment 288: The AAV vector according to any one of embodiments 16-287, wherein said peptide insert comprises or consists of the amino acid sequence IGSVGKL (SEQ ID NO:273).

Embodiment 289: The AAV vector according to any one of embodiments 16-288, wherein said peptide insert comprises or consists of the amino acid sequence SHEVVRA (SEQ ID NO:274).

Embodiment 290: The AAV vector according to any one of embodiments 16-289, wherein said peptide insert comprises or consists of the amino acid sequence EVGHIRG (SEQ ID NO:275).

Embodiment 291: The AAV vector according to any one of embodiments 16-290, wherein said peptide insert comprises or consists of the amino acid sequence GLRGVSV (SEQ ID NO:276).

Embodiment 292: The AAV vector according to any one of embodiments 1-291, wherein said vector comprises a serotype selected from the group consisting of AAV1, AAV2, AAV3, AAV4, AAV5, AAV6, AAV7, AAV8, AAV9, AAV10, and AAVrh74.

Embodiment 293: The AAV vector of embodiment 292, wherein said vector comprises a serotype selected from the group consisting of AAV6, AAVrh74, and AAV9.

Embodiment 294: The AAV vector of embodiment 293, wherein said vector comprises an AAV9 serotype.

Embodiment 295: The AAV vector according to any one of embodiments 1-294, wherein said peptide insert is disposed in viral protein 1 (VP1).

Embodiment 296: The AAV vector of embodiment 295, wherein said peptide insert is disposed in in variable surface region VIII (VR-VIII) of viral protein 1.

Embodiment 297: The AAV vector of embodiment 296, wherein said peptide insert is disposed between residues 581-593 of viral protein 1.

Embodiment 298: The AAV vector of embodiment 297, wherein said peptide insert is disposed between residues 585-592 of viral protein 1.

Embodiment 299: The AAV vector of embodiment 298, wherein said peptide insert is disposed between residues 589-590 of viral protein 1.

Embodiment 300: The AAV vector according to any one of embodiments 1-299, wherein said system further comprises a nucleic acid that encodes a construct that is to be delivered to a muscle cell and/or to a muscle stem cell, where said construct is flanked by AAV inverted terminal repeat (ITR) sequences.

Embodiment 301: The AAV vector of embodiment 300, wherein said construct comprises a gene/cDNA that is to be introduced into said muscle cell and/or muscle stem cell.

Embodiment 302: The AAV vector of embodiment 301, wherein said gene/cDNA comprises a gene or cDNA selected for the treatment of a disease that impacts skeletal muscle or heart.

Embodiment 303: The AAV vector according to any one of embodiments 301-302, wherein said gene/cDNA comprises a gene or cDNA selected for the treatment of a skeletal muscle disorder selected from the group consisting of Limb girdle muscular dystrophy (LGMD), myofibrillar myopathy, myotubular myopathy, BODY, inclusion body myopathy, and vacuolar myopathy.

Embodiment 304: The AAV vector of embodiment 303, wherein said skeletal muscle disorder comprises limb girdle muscular dystrophy (LGMD).

Embodiment 305: The AAV vector according to any one of embodiments 303-304, wherein said gene comprise a gene selected from the group consisting of CAPN3, DYSF, TRIM32, ACTA1, MTM1, PAX7, DES, and LAMP2.

Embodiment 306: The AAV vector of embodiment 305, wherein said gene comprises CAPN3.

Embodiment 307: The AAV vector of embodiment 306, wherein said gene comprises a full-length CAPN3 cDNA.

Embodiment 308: The AAV vector of embodiment 307, wherein said gene comprises a UTR-deleted, Kozak-optimized CAPN3 cDNA.

Embodiment 309: The AAV vector of embodiment 300, wherein said construct comprises a nucleic acid that encodes a gene-editing protein.

Embodiment 310: The AAV vector of embodiment 309, wherein said gene editing protein comprises a component of a CRISPR/Cas system, a CRISPR base editor, a TALEN, and a zinc finger protein.

Embodiment 311: The AAV vector of embodiment 310, wherein said construct encodes a base editor.

Embodiment 312: The AAV vector of embodiment 311, wherein said gene editor comprises a cytidine base editor that provides a C to T conversion or an adenine base editor that provides an A to G conversion.

Embodiment 313: The AAV vector of embodiment 310, wherein said construct encodes a CRISPR protein and a guide RNA.

Embodiment 314: The AAV vector of embodiment 313, wherein said construct encodes an RNA to direct said CRISPR protein to cleave exons 45-55 of the DMD gene.

Embodiment 315: The AAV vector according to any one of embodiments 1-314, wherein vector has a reduced tropism for liver as compared to the same vector lacking said inserted peptide.

Embodiment 316: A nucleic acid encoding an adeno-associated virus (AAV) capsid protein comprising a peptide insert that improves the ability of an AAV vector comprising said capsid protein said vector to transduce a muscle cell and/or a muscle stem cell.

Embodiment 317: The nucleic acid of embodiment 316, wherein said nucleic acid encodes a capsid protein of the AAV vector according to any one of embodiments 1-299.

Embodiment 318: The nucleic acid according to any one of embodiments 316-317, wherein said nucleic acid comprises a plasmid.

Embodiment 319: A nucleic acid encoding an adeno-associated virus (AAV) capsid protein comprising a peptide insert that improves the ability of an AAV vector comprising said capsid protein said vector to transduce a muscle cell and/or a muscle stem cell as compared to the same strain AAV lacking said insert.

Embodiment 320: The nucleic acid of embodiment 319, wherein said peptide insert ranges in length from about 7 up to about 15 amino acids.

Embodiment 321: The nucleic acid of embodiment 320, wherein said peptide insert ranges in length from about 7 up to about 10 amino acids, or up to about 9 amino acids.

Embodiment 322: The nucleic acid of embodiment 321, wherein said peptide insert has a length of seven amino acids.

Embodiment 323: The nucleic acid according to any one of embodiments 319-322, wherein said peptide insert comprises or consists of an amino acid sequence selected from the group consisting of GGMRRDP (SEQ ID NO:1), GVAISGF (SEQ ID NO:2), GQKWNVD (SEQ ID NO:3), GKVGVWA (SEQ ID NO:4), GAFELHN (SEQ ID NO:5), QVLRPTY (SEQ ID NO:6), LGRVHSL (SEQ ID NO:7), LYANVSP (SEQ ID NO:8), LSLGPTV (SEQ ID NO:9), LSLSLAT (SEQ ID NO:10), LNSSSYG (SEQ ID NO:11), LLRNSTA (SEQ ID NO:12), FHSSASA (SEQ ID NO:13), FVGDTVA (SEQ ID NO:14), FGRGSSG (SEQ ID NO:15), WDKAATF (SEQ ID NO:16), AEAPILL (SEQ ID NO:17), AFHGLLA (SEQ ID NO:18), AGYSIVH (SEQ ID NO:19), ATWNKSQ (SEQ ID NO:20), SVHSDYR (SEQ ID NO:21), SGRNSDL (SEQ ID NO:22), SVFVPMR (SEQ ID NO:23), VHGQYVE (SEQ ID NO:24), VERGASV (SEQ ID NO:25), VSEMKER (SEQ ID NO:26), GTPSHYG (SEQ ID NO:27), ILSKGAL (SEQ ID NO:28), GMGTVAR (SEQ ID NO:29), RVGHFSA (SEQ ID NO:30), PSMGFTP (SEQ ID NO:31), ALMTGKL (SEQ ID NO:32), PSMTRQT (SEQ ID NO:33), VERGASV (SEQ ID NO:34), KLIGRSP (SEQ ID NO:35), AEAPILL (SEQ ID NO:36), NPNLSLG (SEQ ID NO:37), TMKMFAS (SEQ ID NO:38), SAYNRAA (SEQ ID NO:39), IASTVYG (SEQ ID NO:40), VDSSRVR (SEQ ID NO:41), MNGLRGT (SEQ ID NO:42), YGNSKVV (SEQ ID NO:43), SAVYTRA (SEQ ID NO:44), LAITVAT (SEQ ID NO:45), FVKANVL (SEQ ID NO:46), GEDIGEH (SEQ ID NO:47), KSQLSYV (SEQ ID NO:48), ITLSGSS (SEQ ID NO:49), AKFPVKV (SEQ ID NO:50), SLTAMRG (SEQ ID NO:51), RSEYVRV (SEQ ID NO:52), VRDLTRV (SEQ ID NO:53), GIGHLAS (SEQ ID NO:54), HESRPPY (SEQ ID NO:55), SSTQKVS (SEQ ID NO:56), FGVSRST (SEQ ID NO:57), MIRGIPS (SEQ ID NO:58), IRQPTSH (SEQ ID NO:59), VSGAVLG (SEQ ID NO:60), RNSDNLS (SEQ ID NO:61), RLASFNS (SEQ ID NO:62), YRIGEGT (SEQ ID NO:63), VHSLGHM (SEQ ID NO:64), VTAVAKV (SEQ ID NO:65), LHHSPSY (SEQ ID NO:66), and ERARSTN (SEQ ID NO:67).

Embodiment 324: The nucleic acid of embodiment 323, wherein said peptide insert comprises or consists of an amino acid sequence selected from the group consisting of

(SEQ ID NO: 1)
GGMRRDP,
(SEQ ID NO: 2)
GVAISGF,
(SEQ ID NO: 3)
GQKWNVD,
(SEQ ID NO: 4)
GKVGVWA,
(SEQ ID NO: 5)
GAFELHN,
(SEQ ID NO: 6)
QVLRPTY,
(SEQ ID NO: 7)
LGRVHSL,
(SEQ ID NO: 8)
LYANVSP,
(SEQ ID NO: 9)
LSLGPTV,
(SEQ ID NO: 10)
LSLSLAT,
(SEQ ID NO: 11)
LNSSSYG,
(SEQ ID NO: 12)
LLRNSTA,
(SEQ ID NO: 13)
FHSSASA,
(SEQ ID NO: 14)
FVGDTVA,
(SEQ ID NO: 15)
FGRGSSG,
(SEQ ID NO: 16)
WDKAATF,
(SEQ ID NO: 17)
AEAPILL,
(SEQ ID NO: 18)
AFHGLLA,
(SEQ ID NO: 19)
AGYSIVH,
(SEQ ID NO: 20)
ATWNKSQ,
(SEQ ID NO: 21)
SVHSDYR,
(SEQ ID NO: 22)
SGRNSDL,
(SEQ ID NO: 23)
SVFVPMR,
(SEQ ID NO: 24)
VHGQYVE,
(SEQ ID NO: 25)
VERGASV,
(SEQ ID NO: 26)
VSEMKER.

Embodiment 325: The nucleic acid of embodiment 323, wherein said peptide insert comprises or consists of an amino acid sequence selected from the group consisting of

(SEQ ID NO: 27)
GTPSHYG,
(SEQ ID NO: 28)
ILSKGAL,
(SEQ ID NO: 29)
GMGTVAR,
(SEQ ID NO: 30)
RVGHFSA,
(SEQ ID NO: 31)
PSMGFTP,
(SEQ ID NO: 32)
ALMTGKL,
(SEQ ID NO: 33)
PSMTRQT,
(SEQ ID NO: 34)
VERGASV,
(SEQ ID NO: 35)
KLIGRSP,
(SEQ ID NO: 36)
AEAPILL,
(SEQ ID NO: 37)
NPNLSLG,
(SEQ ID NO: 38)
TMKMFAS,
(SEQ ID NO: 39)
SAYNRAA,
(SEQ ID NO: 40)
IASTVYG,
(SEQ ID NO: 41)
VDSSRVR,
(SEQ ID NO: 42)
MNGLRGT,
(SEQ ID NO: 43)
YGNSKVV,
(SEQ ID NO: 44)
SAVYTRA,
(SEQ ID NO: 45)
LAITVAT,
(SEQ ID NO: 46)
FVKANVL,
(SEQ ID NO: 47)
GEDIGEH,
(SEQ ID NO: 48)
KSQLSYV,
(SEQ ID NO: 49)
ITLSGSS,
(SEQ ID NO: 50)
AKFPVKV,
(SEQ ID NO: 51)
SLTAMRG,
(SEQ ID NO: 52)
RSEYVRV.

Embodiment 326: The nucleic acid of embodiment 323, wherein said peptide insert comprises or consists of an amino acid sequence selected from the group consisting of

(SEQ ID NO: 53)
VRDLTRV,
(SEQ ID NO: 54)
GIGHLAS,
(SEQ ID NO: 55)
HESRPPY,
(SEQ ID NO: 56)
SSTQKVS,
(SEQ ID NO: 57)
FGVSRST,
(SEQ ID NO: 58)
MIRGIPS,
(SEQ ID NO: 59)
IRQPTSH,
(SEQ ID NO: 60)
VSGAVLG,
(SEQ ID NO: 61)
RNSDNLS,
(SEQ ID NO: 62)
RLASENS,
(SEQ ID NO: 63)
YRIGEGT,
(SEQ ID NO: 64)
VHSLGHM,
(SEQ ID NO: 65)
VTAVAKV,
(SEQ ID NO: 66)
LHHSPSY,
(SEQ ID NO: 67)
ERARSTN,
(SEQ ID NO: 68)
GMGLTVA,
(SEQ ID NO: 69)
LNSSSYG,
(SEQ ID NO: 70)
GYKQVSN,
(SEQ ID NO: 71)
GKASKDT,
(SEQ ID NO: 72)
QLSSRGA,
(SEQ ID NO: 73)
VGTRFTS,
(SEQ ID NO: 74)
QRSSNGP,
(SEQ ID NO: 75)
YKTQGLA,
(SEQ ID NO: 76)
RDSMVNP,
(SEQ ID NO: 77)
RVSSNLS,
and
(SEQ ID NO: 78)
DGTYKGA

Embodiment 327: The nucleic acid according to any one of embodiments 319-326, wherein said capsid protein comprises a capsid protein of an AAV serotype selected from the group consisting of AAV1, AAV2, AAV7, and AAV9.

Embodiment 328: The nucleic acid of embodiment 327, wherein said capsid protein comprises a capsid protein of an AAV9 serotype.

Embodiment 329: The nucleic acid according to any one of embodiments 319-328, wherein said capsid protein comprises viral protein 1 (VP1).

Embodiment 330: The nucleic acid of embodiment 329, wherein said peptide insert is disposed in insertion in variable surface region VIII (VR-VIII) of said viral protein 1.

Embodiment 331: The nucleic acid of embodiment 330, wherein said peptide insert is disposed between residues 581-593 of viral protein 1.

Embodiment 332: The nucleic acid of embodiment 331, wherein said peptide insert is disposed between residues 585-592 of viral protein 1.

Embodiment 333: The nucleic acid of embodiment 332, wherein said peptide insert is disposed between residues 589-590 of viral protein 1.

Embodiment 334: The nucleic acid according to any one of embodiments 319-333, wherein said nucleic acid is operably linked to a promoter.

Embodiment 335: The nucleic acid of embodiment 334, wherein said promoter comprises a constitutive promoter.

Embodiment 336: The nucleic acid of embodiment 334, wherein said promoter comprises an inducible promoter.

Embodiment 337: The nucleic acid according to any one of embodiments 319-336, wherein said nucleic acid comprises a plasmid.

Embodiment 338: An adeno-associated virus (AAV) packaging system for producing a pseudotyped AAV vector having tropism for muscle cells and/or muscle stem cells said system comprising:
a plasmid that encodes a capsid protein wherein said plasmid comprises a nucleic acid sequence according to any one of embodiments 316-337; and
a plasmid that comprises AAV helper genes.

Embodiment 339: The packaging system of embodiment 338, wherein said plasmid that encodes a capsid protein, comprises an AAV Rep gene.

Embodiment 340: The packaging system according to any one of embodiments 338-339, wherein said plasmid that comprises AAV helper genes encodes AAV genes E4, E2A, and Va.

Embodiment 341: The packaging system according to any one of embodiments 338-340, wherein said system further comprises a packaging cell.

Embodiment 342: The packaging system of embodiment 341, wherein said packaging cell comprises a cell selected from the group consisting of HEK293, HEK293T, TE671, HT1080, 3T3, K562, 3T3, U937, and H9.

Embodiment 343: The packaging system of embodiment 342, wherein said packaging cell comprises an HEK293T cell.

Embodiment 344: The packaging system according to any one of embodiments 338-343, wherein said system further comprises a nucleic acid that encodes a construct that is to be delivered to a muscle cell and/or to a muscle stem cell, where said construct is flanked by AAV inverted terminal repeat (ITR) sequences.

Embodiment 345: The packaging system of embodiment 344, wherein said construct comprises a gene/cDNA that is to be introduced into said muscle cell and/or muscle stem cell.

Embodiment 346: The packaging system of embodiment 345, wherein said gene/cDNA comprises a gene or cDNA selected for the treatment of a skeletal muscle disorder selected from the group consisting of Limb girdle muscular dystrophy (LGMD), myofibrillar myopathy, myotubular myopathy, BODY, inclusion body myopathy, and vacuolar myopathy.

Embodiment 347: The packaging system of embodiment 346, wherein said skeletal muscle disorder comprises limb girdle muscular dystrophy (LGMD).

Embodiment 348: The packaging system according to any one of embodiments 346-347, wherein said gene comprise a gene selected from the group consisting of CAPN3, DYSF, TRIM32, ACTA1, MTM1, PAX7, DES, and LAMP2.

Embodiment 349: The packaging system of embodiment 348, wherein said gene comprises CAPN3.

Embodiment 350: The packaging system of embodiment 349, wherein said gene comprises a full-length CAPN3 cDNA.

Embodiment 351: The packaging system of embodiment 349, wherein said gene comprises a UTR-deleted, Kozak-optimized CAPN3 cDNA.

Embodiment 352: The packaging system of embodiment 344, wherein said construct comprises a nucleic acid that encodes a gene-editing protein.

Embodiment 353: The packaging system of embodiment 352, wherein said gene editing protein comprises a component of a CRISPR/Cas system, a TALEN, and a zinc finger protein.

Embodiment 354: The packaging system of embodiment 353, wherein construct encodes a CRISPR protein and a guide RNA.

Embodiment 355: The packaging system of embodiment 354, wherein said construct encodes an RNA to direct said CRISPR protein to cleave exons 45-55 of the DMD gene.

Embodiment 356: A packaging cell for packaging a pseudotyped AAV vector having tropism for muscle cells and/or muscle stem cells, said packaging cell is a mammalian cell comprising a nucleic acid according to any one of embodiments 316-337 operably linked to a promoter and capable of expressing said capsid protein.

Embodiment 357: The packaging cell of embodiment 356, wherein said nucleic acid comprises a plasmid disposed episomally in said cell.

Embodiment 358: The packaging cell of embodiment 356, wherein said nucleic acid is integrated into the genome of said cell.

Embodiment 359: The packaging cell according to any one of embodiments 356-358, wherein said cell further comprises all the components required for encapsidation of an adeno-associated virus.

Embodiment 360: The packaging cell according to any one of embodiments 356-358, wherein said cell further comprises nucleic acid(s) encoding encodes AAV genes E4, E2A, and Va.

Embodiment 361: The packaging cell according to any one of embodiments 356-360, wherein said mammalian cell is selected from the group consisting of HEK293, HEK293T, TE671, HT1080, 3T3, K562, 3T3, U937, and H9.

Embodiment 362: The packaging cell of embodiment 361, wherein said cell is a HEK293T cell.

Embodiment 363: A method of producing an AAV vector that has a tropism to a muscle cell and/or to a muscle stem cell, said method comprising: providing:
an adeno-associated virus (AAV) packaging system according to any one of embodiments 338-340;
a nucleic acid encoding a construct that is to be delivered into a muscle cell and/or a muscle stem cell where said construct is flanked by ITR sequences; and
a helper cell; where said helper cells packages said nucleic acid encoding a construct into a pseudotype AAV viral capsid that has a tropism to muscle cells and/or to muscle stem cells.

Embodiment 364: A method of producing an AAV vector that has a tropism to a muscle cell and/or to a muscle stem cell, said method comprising:
transducing a packaging cell according to any one of embodiments 356-362 with a nucleic acid encoding a construct that is to be delivered into a muscle cell and/or a muscle stem cell where said construct is flanked by AAV ITR sequences;
whereby said packaging cells packages said construct into an AAV capsid to produce an AAV virion that has a tropism to a muscle cell and/or to a muscle stem cell.

Embodiment 365: A method of introducing a nucleic acid construct into a muscle cell or a muscle stem cell in a mammal, said method comprising:
providing an AAV vector according to any one of embodiments 1-299, wherein said AAV vector comprises a nucleic construct that is to be delivered into a muscle cell and/or muscle stem cell; and
   administering said vector to said mammal in an amount effective to transduce a muscle cell and/or a muscle stem cell in said mammal.

Embodiment 366: The method of embodiment 365, wherein said construct comprises a gene or cDNA selected for the treatment of a skeletal muscle disorder selected from the group consisting of Limb girdle muscular dystrophy (LGMD), myofibrillar myopathy, myotubular myopathy, BODY, inclusion body myopathy, and vacuolar myopathy.

Embodiment 367: The method of embodiment 366, wherein said skeletal muscle disorder comprises limb girdle muscular dystrophy (LGMD).

Embodiment 368: The method according to any one of embodiments 366-367, wherein said gene comprise a gene selected from the group consisting of CAPN3, DYSF, TRIM32, ACTA1, MTM1, PAX7, DES, and LAMP2.

Embodiment 369: The method of embodiment 368, wherein said gene comprises CAPN3.

Embodiment 370: The method of embodiment 369, wherein said gene comprises a full-length CAPN3 cDNA.

Embodiment 371: The method of embodiment 370, wherein said gene comprises a UTR-deleted, Kozak-optimized CAPN3 cDNA.

Embodiment 372: The method of embodiment 365, wherein said construct comprises a nucleic acid that encodes a gene-editing protein.

Embodiment 373: The method of embodiment 372, wherein said gene editing protein comprises a component of a CRISPR/Cas system, a TALEN, and a zinc finger protein.

Embodiment 374: The method of embodiment 373, wherein construct encodes a CRISPR protein and a guide RNA.

Embodiment 375: The method of embodiment 374, wherein said construct encodes an RNA to direct said CRISPR protein to cleave exons 45-55 of the DMD gene.

### BRIEF DESCRIPTION OF THE DRAWINGS

FIG. 1, panels A-C, illustrates a CRISPR/Cas9 gene-editing platform targeted to delete exons 45-55 and convert DMD to BMD. Panel A) Region of the human DMD gene targeted with a single pair of gRNAs (lightning bolts) to delete exons 45-55 and reframe the DMD gene. Panel B) Immunofluorescent images of healthy, DMD, and BMD human skeletal muscle samples stained for dystrophin (green) and DAPI (blue). Panel C) Humanized dystrophic mouse model null for both human DMD and mouse DMD genes.

FIG. 2, panels A-B, shows that Systemic delivery of AAV9-CRISPR restores dystrophin expression in hDMDdel45 mdx mice. Panel A) Immunofluorescent images of heart sections from hDMDdel45 mdx mice treated with HBSS (vehicle) or AAV9-CRISPR stained with Laminin (white), dystrophin (red) and revertant dystrophin-positive (green) fibers. Panel B) Western blots probed for dystrophin using heart samples from HBSS or AAV9-CRISPR treated mice and wild type hDMDmdx mice as a control for dystrophin.

FIG. 3, panels A-B, shows AAV9-CRISPR targeting to dystrophic MuSCs using the hDMDdel45 mdx mouse and, in particular, shows that AAV9-mCherry inefficiently targets dystrophic muscle stem cells. Panel A) Experimental design to dose hDMDdel45 mdx neonates and adults with AAV9-CRISPR and assess MuSC targeting. Panel B) Representative FACS plots from neonates i.p. injected with AAV9-CRISPR/mCherry to isolate Lin-α7-Integrin+ CD34+ MuSCs and assess targeting.

FIG. 4, panels A-B, shows that AAV9-CRISPR does not efficiently target adult α7-Integrin+/CD34+ MuSC. Panel A) PCR using primers to detect exons 45-55 deletion (purple arrows) region of DMD gene (see FIG. 1, panel A) isolated from α7-Integrin+/CD34+MuSCs, single α7-Integrin+ cells (MuSC-like properties), and single CD34+ cells (non MuSCs). Neonate MuSCs are from two independent experiments. Panel B) Sample sequence trace showing rejoining of introns 44 and 55 in MuSC after CRISPR editing.

FIG. 5. Scheme 1 illustrates how novel MuSC-tropic AAV serotypes may provide a long-lasting supply of MuSC and may serve as a sustainable therapy for DMD.

FIG. 6, panels A-C, illustrates the generation of an AAV9 peptide-display library. Panel A) Schematic of the AAV9 peptide-display library vector containing randomized 21nt (NNK)₇ (SEQ ID NO:277) sequences inserted into the capsid reading frame. Panel B) Production of AAV9 peptide-display library using HEK293T cells. Panel C) Examples of successful insertion of (NNK)₇ (SEQ ID NO:277) sequences into the AAV library plasmid demonstrating diversity of the 7-mer amino acid peptide sequence. Clone 1: SEQ ID NO:278; Clone 2: SEQ ID NO:279; Clone 3: SEQ ID NO:280; Clone 4: SEQ ID NO:281; Clone 5: SEQ ID NO:282; Clone 6: SEQ ID NO:283.

FIG. 7 Scheme 2 illustrates an experimental design for AAV9 peptide-display library screening in vivo.

FIG. 8, panels A-D, illustrates AAV variants detected in heart, differentiated skeletal muscle, and muscle stem cells. Panel A) PCR strategy to detect the presence of viral library in muscle tissues, heart, and MuSCs. Panel B) Representative FACS plots showing isolation of Lin-α7-Integrin+ CD34+ MuSCs. Panel C) Gels showing presence of library 21nt (NNK)₇ (SEQ ID NO:277) insert, numbers represent individual mice. Panel D) Representative sequence traces highlighting the randomized 21nt (NNK)₇ (SEQ ID NO:277) oligonucleotide detected in cardiac muscle, skeletal muscles, and muscle stem cells. Heart: SEQ ID NO:284; Triceps SEQ ID NO:285; Diaphragm SEQ ID NO:286; MuSCs: SEQ ID NO:287.

### DETAILED DESCRIPTION

In certain embodiments pseudotyped adeno-associated viral (AAV) vectors are provided that are able to transduce certain tissues or cells better than others. In particular, we have identified muscle stem cell targeting pseudotyped AAV. While others have claimed that AAV9 can target muscle stem cells, we have not been able to replicate the findings bringing into question efficacy of AAV9 for targeting muscle stem cells. In view of this AAV serotypes were sought and identified that have a tropism for (e.g., can effectively target and transduce) muscle stem cells (MuSCs). Additionally, AAV serotypes were created that have a tropism for cardiac and/or for skeletal muscle cells. In certain embodiments the pseudotyped AAV vectors described herein detarget (e.g., show a reduced or absent tropism for) liver. In certain embodiments the pseudotyped AAV vectors described herein show a preferential tropism for skeletal muscle over cardiac muscle.

Accordingly in various embodiments, uses pseudotyped AAV vectors that transduce muscle cells and/or muscle stem cells (MuSC) are provided. In certain embodiments uses of such AAV vectors are provided. Such uses include, but are not limited to the delivery of nucleic acid constructs (e.g., CRISPR/Cas, TALENs, zinc finger proteins) or genes/CDNs (e.g., transgenes). In various embodiments such constructs can be used for, inter alia, the treatment of various myopathies (e.g., Limb girdle muscular dystrophy (LGMD), myofibrillar myopathy, myotubular myopathy, inclusion body myopathy, vacuolar myopathy, and the like).

In various embodiments packaging systems and packaging cells are provided for the production of AAV vectors (virions) that have a tropism for muscle cells and/or for muscle stem cells. In certain embodiments the virions have a reduced tropism for liver cells as compared to the same vector lacking a peptide insert described herein.

### Pseudotyped AAV.

As described in Example 1, libraries comprising AAV with a 7 amino acid peptide insert in a capsid protein (e.g., VP1) were screened to identify sequences that produce an AAV pseudotyped to transduce muscle cells and/or muscle stem cells. Accordingly, in various embodiments an adeno-associated virus (AAV) vector pseudotyped to transduce muscle and/or muscle stem cells is provided where the vector comprises a capsid protein comprising a peptide insert that improves the ability of the vector to transduce a muscle cell and/or a muscle stem cell. In certain embodiments the peptide insert ranges in length from about 7 up to about 15 amino acids, or ranges in length from about 7 up to about 10 amino acids, or up to about 9 amino acids. In certain embodiments the peptide insert has a length of seven amino acids.

In certain embodiments the peptide insert produces a pseudotyped AAV vector that is capable of transducing heart muscle. Illustrative, but non-limiting peptide inserts that produce a pseudotyped AAV vector that transduces heart muscle are shown in Table 1.

TABLE 1
Illustrative peptide inserts that produce AAV that
transduce heart muscle.

| Peptide Seq | SEQ ID NO | Peptide Seq | SEQ ID NO |
|---|---|---|---|
| GGMRRDP | 1 | GVAISGF | 2 |
| GQKWNVD | 3 | GKVGVWA | 4 |
| GAFELHN | 5 | QVLRPTY | 6 |
| LGRVHSL | 7 | LYANVSP | 8 |
| LSLGPTV | 9 | LSLSLAT | 10 |
| LNSSSYG | 11 | LLRNSTA | 12 |
| FHSSASA | 13 | FVGDTVA | 14 |
| FGRGSSG | 15 | WDKAATF | 16 |
| AEAPILL | 17 | AFHGLLA | 18 |
| AGYSIVH | 19 | ATWNKSQ | 20 |
| SVHSDYR | 21 | SGRNSDL | 22 |
| SVFVPMR | 23 | VHGQYVE | 24 |
| VERGASV | 25 | VSEMKER | 26 |
| INHNSHI | 27 | GQSNISR | 28 |
| RQTNQIE | 29 | GPVVQGA | 30 |
| VSGSSFG | 31 | DVSLTRM | 32 |
| NGQGILK | 33 | SGDAYRA | 34 |
| FNNGIHS | 35 | SSYSVSL | 36 |
| ESGNIRP | 37 | VSGGQTQ | 38 |
| IVGSGSA | 39 | VSSQGTS | 40 |
| GSTYENS | 41 | NGSTSPV | 42 |
| EGKSSPT | 43 | VGQGISL | 44 |
| AGIGQAG | 45 | ITSPSVS | 46 |
| EHVEGRR | 47 | LDGVSHM | 48 |
| GGGTLHH | 49 | HESRPPY | 50 |
| SNHDSRV | 51 | SGSGSFM | 52 |
| VGSLYQP | 53 | GQDNRAL | 54 |
| RSEYNSV | 55 | TGQTVLG | 56 |
| GGDAARY | 57 | RGSNDLP | 58 |
| VASGTVL | 59 | GDHNTIK | 60 |
| NARDSGF | 61 | PGNINNN | 62 |
| SCGDSCR | 63 | FGSVHSP | 64 |
| SLGGLGN | 65 | AADAVRV | 66 |
| VIAQGMS | 67 | NHSSGAM | 68 |
| LRDNSAF | 69 | LGSGALA | 70 |
| SHEVVRA | 71 | FNISANE | 72 |
| ARSQEVP | 73 | AGNITSF | 74 |
| TCNSVGC | 75 | GGGSSSA | 76 |
| YDQVSHP | 77 | VHGISPL | 78 |
| TGLNYTT | 79 | QDRNYGV | 80 |
| VSEINRG | 81 | QRSSNGP | 82 |
| SSTGTWV | 83 | QLPNNLS | 84 |
| YNGAIHH | 85 | MRNTESS | 86 |
| GASPSSI | 87 | DGTYKGA | 88 |
| GQGCTQC | 89 | GAHPTGG | 90 |
| SNYNVTG | 91 | SANVESP | 92 |
| AHSGGTY | 93 | GENAGRA | 94 |
| IGCDYPC | 95 | ISLPNSI | 96 |
| NGINGAS | 97 | GPSSLRE | 98 |
| VQSGNYV | 99 | GGYSETS | 100 |
| LRVGNVE | 101 | GMGTVAR | 102 |
| VQMSGYL | 103 | QNMVVTS | 104 |
| THGLPIV | 105 | GFNHGAN | 106 |
| NSYSVSP | 107 | SMLGGVA | 108 |
| YGVDMGL | 109 | VIGHGSA | 110 |
| SGLDKAP | 111 | DVKNGGR | 112 |
| IGNGGLI | 113 | QVGFPTP | 114 |
| LSSNANL | 115 | TNLHVNL | 116 |
| SYIGVAA | 117 | STVNGNQ | 118 |
| TNRDAQF | 119 | LHHSPSY | 120 |
| TTYNSSA | 121 | PSSQPLP | 122 |
| RDSMVNP | 123 | SLHSPQL | 124 |
| VQVGTGT | 125 | RENPSGM | 126 |
| GVPKTHD | 127 | VDQGRLS | 128 |
| FDKGAVI | 129 | VSRSGGV | 130 |
| INMQSMA | 131 | GTASSRP | 132 |
| EVGHIRG | 133 | RDVSSGG | 134 |
| ITLSGSS | 135 | MNGLRGT | 136 |
| RSQEINL | 137 | YGTVSVP | 138 |
| LGSSGVS | 139 | PGSYVSS | 140 |
| ASNLGTY | 141 | GSEGGSM | 142 |
| IASGNSV | 143 | ATAGAII | 144 |
| LSSMGQL | 145 | GEKFMGV | 146 |

It will be recognized that AAV comprising such inserts in a capsid protein can be utilized to effectively deliver nucleic acid constructs to cardiac muscle. Accordingly, in certain embodiments, such AAV find utility in the treatment of various cardiac disorders (e.g., inherited cardiomyopathies and heart failure (see, e.g., www.ncbi.nlm.nih.gov/pmc/articles/PMC5544589/)). In certain embodiments the peptide insert comprises one of the sequences shown in Table 1. In certain embodiments capsid protein comprise two or more (e.g., 2, 3, 4, 5, etc.) of the peptide sequences shown in Table 1.

In certain embodiments the peptide insert produces a pseudotyped AAV vector that is capable of transducing skeletal muscle, Illustrative, but non-limiting peptide inserts that produce a pseudotyped AAV vector that transduces skeletal muscle are shown in Table 2.

TABLE 2
Illustrative peptide inserts that produce AAV
that transduce skeletal muscle.

| Peptide | | Peptide | |
|---|---|---|---|
| Sequence | SEQ ID NO | Sequence | SEQ ID NO |
| GTPSHYG | 147 | ILSKGAL | 148 |
| GMGTVAR | 149 | RVGHFSA | 150 |
| PSMGFTP | 151 | ALMTGKL | 152 |
| PSMTRQT | 153 | VERGASV | 154 |
| KLIGRSP | 155 | AEAPILL | 156 |
| NPNLSLG | 157 | TMKMFAS | 158 |
| SAYNRAA | 159 | IASTVYG | 160 |
| VDSSRVR | 161 | MNGLRGT | 162 |
| YGNSKVV | 163 | SAVYTRA | 164 |
| LAITVAT | 165 | FVKANVL | 166 |
| GEDIGEH | 167 | KSQLSYV | 168 |
| ITLSGSS | 169 | AKFPVKV | 170 |
| SLTAMRG | 171 | RSEYVRV | 172 |
| WNGELVA | 173 | GQSNISR | 174 |
| AEAPILL | 175 | YGTVSVP | 176 |
| GLLNSSP | 177 | YESSRTA | 178 |
| ESGNIRP | 179 | LNSSSYG | 180 |
| DNRTVNV | 181 | SGSGSFM | 182 |
| TRLNTVQ | 183 | VSGGQTQ | 184 |
| DGTYKGA | 185 | RQTNQIE | 186 |
| GTASSRP | 187 | LGSSGVS | 188 |
| AHSGGTY | 189 | SHEVVRA | 190 |
| YISESRP | 191 | SLGGLGN | 192 |
| NSYSVSP | 193 | GSITYTP | 194 |
| NGINGAS | 195 | VSGSSFG | 196 |
| EVRVANA | 197 | SGRNSDL | 198 |
| MTGLRGT | 199 | GFNHGAN | 200 |
| GENAGRA | 201 | QNMVVTS | 202 |
| GASPSSI | 203 | YVNKSQA | 204 |
| GTPSQYG | 205 | NHSSGAM | 206 |
| TNLNLRE | 207 | GRISGGS | 208 |

It will be recognized that AAV comprising such inserts in a capsid protein can be utilized to effectively deliver nucleic acid constructs to skeletal muscle. In certain embodiments the AAV deliver nucleic acid constructs preferentially to skeletal muscle over heart muscle. Accordingly, in certain embodiments, such AAV find utility in the treatment of various skeletal muscle disorders (e.g., Limb girdle muscular dystrophy (LGMD), myofibrillar myopathy, myotubular myopathy, inclusion body myopathy, vacuolar myopathy, and the like). In certain embodiments the peptide insert consists or comprises one of the sequences shown in Table 2. In certain embodiments capsid protein comprise two or more (e.g., 2, 3, 4, 5, etc.) of the peptide sequences shown in Table 2. In certain embodiments the capsid protein comprises at least one amino acid sequence shown in Table 1, and at least one amino acid sequence shown in Table 2.

In certain embodiments the peptide insert produces a pseudotyped AAV vector that is capable of transducing muscle stem cells. Illustrative, but non-limiting peptide inserts that produce a pseudotyped AAV vector that transduces muscle stem cells are shown in Table 3.

TABLE 3
Illustrative peptide inserts that produce AAV
that transduce muscle stem cells.

| Peptide | | Peptide | |
|---|---|---|---|
| sequence | SEQ ID NO | Sequence | SEQ ID NO |
| VRDLTRV | 209 | GIGHLAS | 210 |
| HESRPPY | 211 | SSTQKVS | 212 |
| FGVSRST | 213 | MIRGIPS | 214 |
| IRQPTSH | 215 | VSGAVLG | 216 |
| RNSDNLS | 217 | RLASENS | 218 |
| YRIGEGT | 219 | VHSLGHM | 220 |
| VTAVAKV | 221 | LHHSPSY | 222 |
| ERARSTN | 223 | GMGLTVA | 224 |
| LNSSSYG | 225 | GYKQVSN | 226 |
| GKASKDT | 227 | QLSSRGA | 228 |
| VGTRFTS | 229 | QRSSNGP | 230 |
| YKTQGLA | 231 | RDSMVNP | 232 |
| RVSSNLS | 233 | DGTYKGA | 234 |
| FGSGALA | 235 | ARSQEVP | 236 |
| VVVVGRP | 237 | LHGARSS | 238 |
| SGQMRGA | 239 | TRLNTVQ | 240 |
| HMHVCVP | 241 | VTNSSRV | 242 |
| NQLPHFK | 243 | VRFGSNP | 244 |
| YISESRP | 245 | VSRSGGV | 246 |
| LSSNANL | 247 | DNRTVNV | 248 |
| LRVGNVE | 249 | GTASSRP | 250 |
| LHGARYS | 251 | TGVNVKS | 252 |
| VTSWTKN | 253 | RGSTMMP | 254 |
| SGSGSFM | 255 | VSGGQTQ | 256 |
| GMGTVAR | 257 | LNPRSSS | 258 |
| SQNLLSN | 259 | YRIGEGT | 260 |
| STVNGNQ | 261 | AADAVRV | 262 |
| YGNSKVV | 263 | VPGEGRV | 264 |
| GGDAARY | 265 | YDQVSHP | 266 |
| GSFHGTV | 267 | GAHPTGG | 268 |
| RDGQSRG | 269 | SLGGLGN | 270 |
| DGTYKGA | 271 | GIGHLAS | 272 |
| IGSVGKL | 273 | SHEVVRA | 274 |
| EVGHIRG | 275 | GLRGVSV | 276 |

It will be recognized that AAV comprising such inserts in a capsid protein can be utilized to effectively deliver nucleic acid constructs to muscle stem cells. Accordingly, in certain embodiments, such AAV find utility in the treatment of a wide variety of muscle cell disorders, including, but not limited to those identified above. In certain embodiments the peptide insert consists or comprises one of the sequences shown in Table 3. In certain embodiments capsid protein comprise two or more (e.g., 2, 3, 4, 5, etc.) of the peptide sequences shown in Table 3. In certain embodiments the capsid protein comprises at least one amino acid sequence shown in Table 1, and at least one amino acid sequence shown in Table 3. In certain embodiments the capsid protein comprises at least one amino acid sequence shown in Table 2, and at least one amino acid sequence shown in Table 3. In certain embodiments the capsid protein comprises at least one amino acid sequence shown in Table 1, one amino acid sequence shown in Table 2, and one amino acid sequence shown in Table 3.

In certain embodiments the AAV vector comprises a serotype selected from the group consisting of AAV1, AAV2, AAV3, AAV4, AAV5, AAV6, AAV7, AAV8, AAV9, AAV10, and AAVrh74. In certain embodiments the AAV vector comprises a serotype selected from the group consisting of AAV6, AAV9, and AAVrh74. In certain embodiments the vector comprises an AAV9 serotype.

In certain embodiments the peptide insert is disposed in viral protein 1 (VP1). In certain embodiments the peptide insert is disposed in the variable surface region VIII (VR-VIII) of viral protein 1. In certain embodiments the peptide insert is disposed between residues 581-593 of viral protein 1, or between residues 585-592 of viral protein 1, or between residues 589-590 of viral protein 1.

As noted above, the AAV vectors described herein can contain a nucleic acid that encodes a construct that is to be delivered to a muscle cell and/or to a muscle stem cell. Typically, the construct is flanked by AAV inverted terminal repeat (ITR) sequences. In certain embodiments the construct comprises a gene/cDNA that is to be introduced into said muscle cell and/or muscle stem cell, while in certain other embodiments, the construct comprises a nucleic acid that encodes a gene-editing protein.

In certain embodiments the construct comprises a gene or cDNA selected for the treatment of a skeletal muscle disorder (e.g., Limb girdle muscular dystrophy (LGMD), myofibrillar myopathy, myotubular myopathy, inclusion body myopathy, vacuolar myopathy, etc.). In certain embodiments the construct comprises a gene or cDNA selected for the treatment of limb girdle muscular dystrophy (LGMD).

In certain embodiments the construct comprise a gene or cDNA selected from the group consisting of CAPN3, DYSF, TRIM32, ACTA1, MTM1, PAX7, DES, and LAMP2. In certain embodiments, e.g., for the treatment of LGMD, the gene construct encodes CAPN3. In certain embodiments the construct comprises a full-length CAPN3 cDNA. In certain embodiments the construct comprises a UTR-deleted, Kozak-optimized CAPN3 cDNA.

In certain embodiments the construct comprises a nucleic acid that encodes a gene-editing protein (e.g., a component of a CRISPR/Cas system, a TALEN, a zinc finger protein, etc.). In certain embodiments the construct encodes a CRISPR protein and a guide RNA. In certain embodiments the construct encodes an RNA to direct said CRISPR protein to cleave exons 45-55 of the DMD gene. Illustrative CRISPR/Cas constructs for the modification of a mutant dystrophin gene, e.g., for the treatment of LGMD are described, for example, in PCT Publication No: PCT/US2017/017255 which is incorporated herein by reference for the CRISPR/Cas constructs described therein.

### Nucleic Acid Encoding Modified AAV Capsid Protein.

In certain embodiments nucleic acids encoding modified AAV capsid proteins are provided. Such nucleic acids find utility, for example, in the preparation of packaging cells to produce AAV vectors having a tropism for muscle cells and/or muscle stem cells, for use in transfection systems (e.g., triple transfection systems) for the production of AAV virions that have a tropism for muscle cells and/or muscle stem cells.

### Packaging Cells and Systems.

### Packaging Cells.

One method for the production of recombinant AAV utilizes packaging cells. In certain embodiments the packaging cells comprise AAV rep/Cap genes as well as the AAV vector DNA. In certain embodiments such cells will comprise a nucleic acid encoding a capsid gene comprising a peptide insert incorporation of which produces an AAV having a tropism for muscle cells and/or muscle stem cells. Such nucleic acids are described above, with respect to pseudotyped AAV vectors. In certain embodiments such packaging cells rely on wild-type adenovirus infection into the packaging cell lines that stably harbor AAV rep/cap genes, as well as the AAV vector DNA.

Thus in certain embodiments, a packaging cell for packaging a pseudotyped AAV vector having tropism for muscle cells and/or muscle stem cells, is provided. In certain embodiments the packaging cell is a mammalian cell comprising a nucleic acid (e.g., a nucleic acid encoding a capsid protein (e.g., VP1) with a peptide insert (e.g., comprising or consisting of one or more amino acid sequences in Tables 1, 2, or 3) as described above, operably linked to a promoter and capable of expressing the capsid protein. In certain embodiments the nucleic acid comprises a plasmid disposed episomally in the cell. In certain embodiments the nucleic acid is integrated into the genome of said cell. In certain embodiments the cell further comprises nucleic acid(s) encoding all of the components required for encapsidation of an adeno-associated virus (e.g., AAV genes E4, E2A, and Va). In certain embodiments the mammalian cell is selected from the group consisting of HEK293, HEK293T, TE671, HT1080, 3T3, K562, 3T3, U937, and H9. In certain embodiments the cell is a HEK293T cell.

In various embodiments methods of utilizing a packaging cell to produce a pseudotyped AAV vector having a tropism for muscle and/or muscle stems cells are provided. In one illustrative, but non-limiting embodiment the method comprises transducing a packaging cell described herein with a nucleic acid encoding a construct that is to be delivered into a muscle cell and/or a muscle stem cell where said construct is flanked by AAV IR sequences. The packaging cells then packages the construct into an AAV capsid to produce an AAV virion that has a tropism to a muscle cell and/or to a muscle stem cell.

### Packaging Systems.

Another illustrative, but non-limiting approach for the production of recombinant AAV, the helper-free method, is based on the adenovirus-free transient transfection of all elements that are required for AAV production in host cells such as HEK293 cells. It typically involves the co-transfection of AAV production cells with 3 plasmids: (1) an AAV2 ITR-containing plasmid carrying the nucleic acid construct (e.g., gene/cDNA) of interest; (2), a plasmid that carries the AAV2 Rep-Cap (in this case a cap comprising one or more of the pseudotype inserts described herein); and (3), a plasmid that provides the helper genes isolated from adenovirus.

Accordingly, in certain illustrative, but non-limiting embodiments, an adeno-associated virus (AAV) packaging system for producing a pseudotyped AAV vector having tropism for muscle cells and/or muscle stem cells is provided. In certain embodiments the system comprises i) a plasmid that encodes a capsid protein wherein said plasmid comprise a nucleic acid sequence encoding an AAV capsid comprising a peptide insert as described herein, and ii) a plasmid that comprises AAV helper genes. In certain embodiments the plasmid that encodes a capsid protein, comprises an AAV Rep gene in addition to the modified Cap gene(s). In certain embodiments the plasmid that comprises AAV helper genes encodes AAV genes E4, E2A, and Va. In certain embodiments the system further comprises a mammalian cell (e.g., HEK293, HEK293T, TE671, HT1080, 3T3, K562, 3T3, U937, H9, and the like). In certain embodiments the cell is an HEK293T cell.

In certain embodiments the system further comprises a nucleic acid that encodes a construct that is to be delivered to a muscle cell and/or to a muscle stem cell, where said construct is flanked by AAV inverted terminal repeat (ITR) sequences. In certain embodiments the construct comprises a gene/cDNA that is to be introduced into said muscle cell and/or muscle stem cell. In certain embodiments the construct encodes a gene editing protein (e.g., CRISPR, TALEN, zinc finger protein, etc.).

### Uses of Pseudotyped AAV.

In certain embodiments the pseudotyped AAV vectors described herein can be used to effectively deliver a nucleic acid construct to a muscle and/or to a muscle stem cell. In certain embodiments the AAV vector shows higher transduction of the muscle cell and/or muscle stem cell than liver cell as compared to the same construct without the peptide insert.

In various embodiments the AAV vectors can deliver transgenes (e.g., genes/cDNAs) or gene editing constructs (e.g., CRISPR/Cas) for the treatment of various muscle cell genetic disorders. In certain embodiments the AAV vectors are used to deliver a gene or cDNA for the treatment of a skeletal muscle disorder including, but not limited to Duchenne Muscular Dystrophy (due to DMD mutations), limb girdle muscular dystrophy (LGMD) (due to CAPN3 mutations), myofibrillar myopathy, myotubular myopathy, BODY, inclusion body myopathy, vacuolar myopathy, and the like. In certain embodiments the skeletal muscle disorder comprises limb girdle muscular dystrophy (LGMD).

In certain embodiments the vectors can deliver a gene such as CAPN3, DYSF, TRIM32, ACTA1, MTM1, PAX7, DES, LAMP2, and the like. Particularly for the treatment of LGMD. In certain embodiments the vector can encode CAPN3. In certain embodiments the vector comprises a full-length CAPN3 cDNA. In certain embodiments the vector comprises a UTR-deleted, Kozak-optimized CAPN3 cDNA.

In certain embodiments the AAV vector comprises a nucleic acid that encodes a gene-editing protein (e.g., a component of a CRISPR/Cas system, a TALEN, a zinc finger protein, and the like). In certain embodiments the AAV vector comprise a construct that encodes a CRISPR protein and a guide RNA. In certain embodiments the construct encodes an RNA to direct said CRISPR protein to cleave exons 45-55 of the DMD gene. In certain embodiments the construct encodes a CRISPR/Cas described, for example, in PCT Publication No: PCT/US2017/017255 which is incorporated herein by reference for the CRISPR/Cas constructs described therein.

The foregoing examples are illustrative and non-limiting. Using the teachings provided herein, numerous other AAV vectors with a tropism for muscle and/or muscle stem cells will be available to one of skill in the art.

### EXAMPLES

The following examples are offered to illustrate, but not to limit the claimed invention.

### Example 1

### Identifying New AAV Serotypes with Muscle Stem Cell Tropism

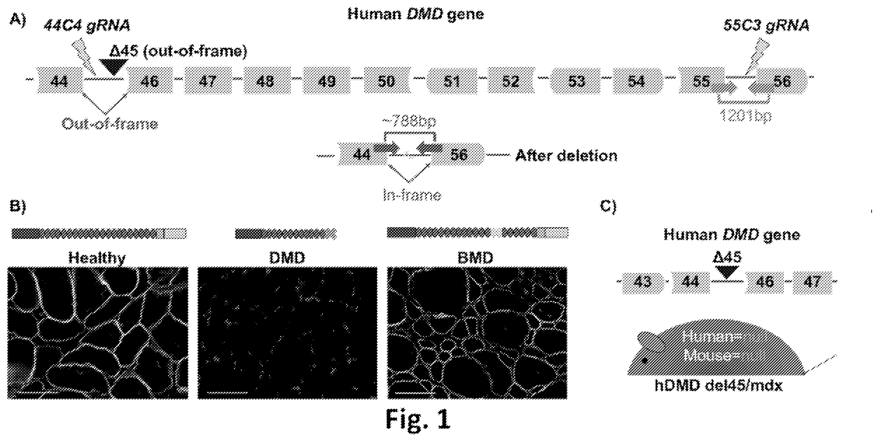

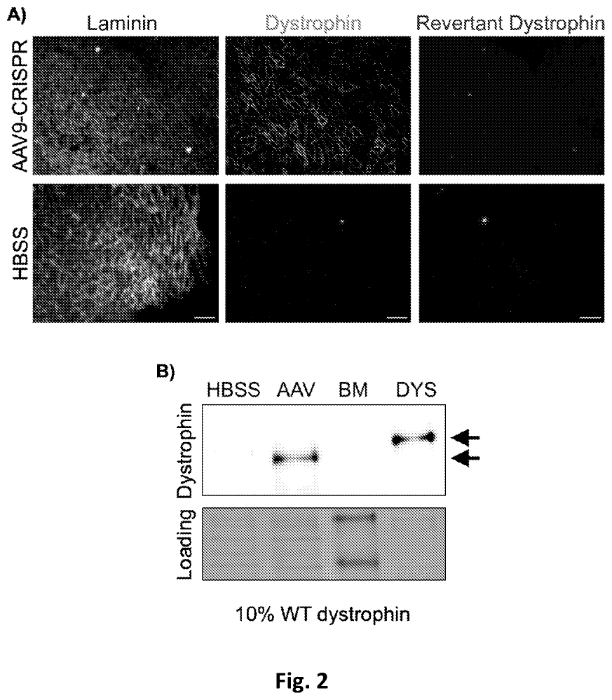

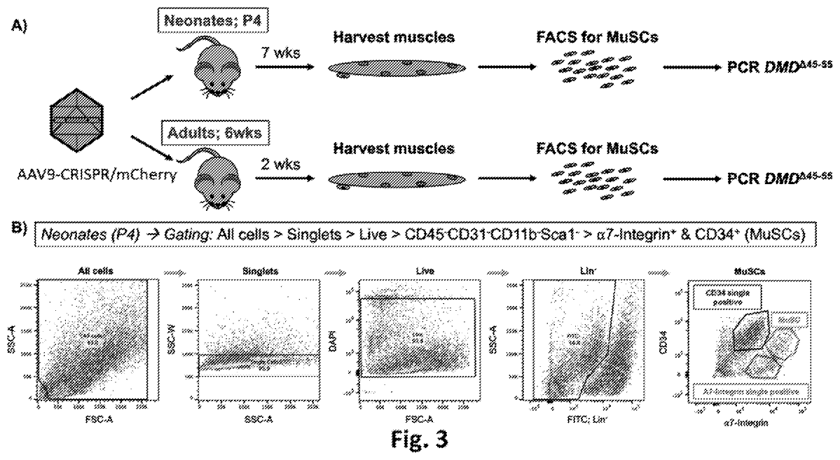

Duchenne muscular dystrophy (DMD) is a monogenic disease caused by out-of-frame mutations in the DMD gene. We have developed a CRISPR/Cas9 gene-editing platform as a therapeutic strategy for DMD. As shown in FIG. 1, panel A, the Crispr/Cas9 targeted a region of the human DMD gene with a single pair of gRNAs to delete exons 45-55 and to thereby reframe the DMD gene. Effective re-framing is illustrated in FIG. 1, panel B. Efficiency of this approach in a mouse model (FIG. 1, panel C) is illustrated in FIG. 2, panels A-B.

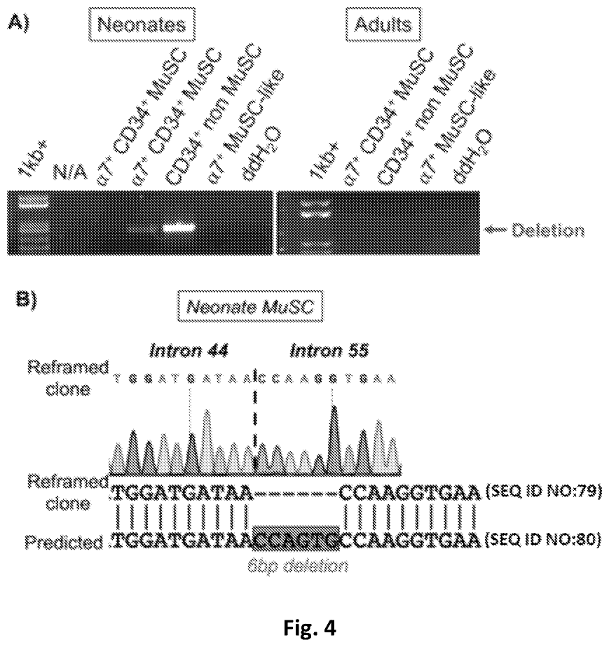

Systemic delivery of CRISPR/Cas9 can be achieved using adeno-associated virus (AAV); however, to achieve a long-lasting therapy, muscle stem cell (MuSC) must be targeted by AAV. We observed that AAV9 can transduce MuSCs and edit the DMD gene in neonates but we do not detect editing in adult mice with our CRISPR platform (see, e.g., FIG. 3, panels A-B, and FIG. 4, panels A-B).

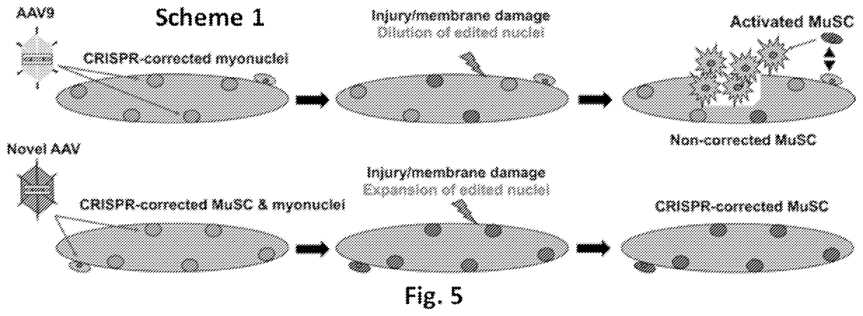

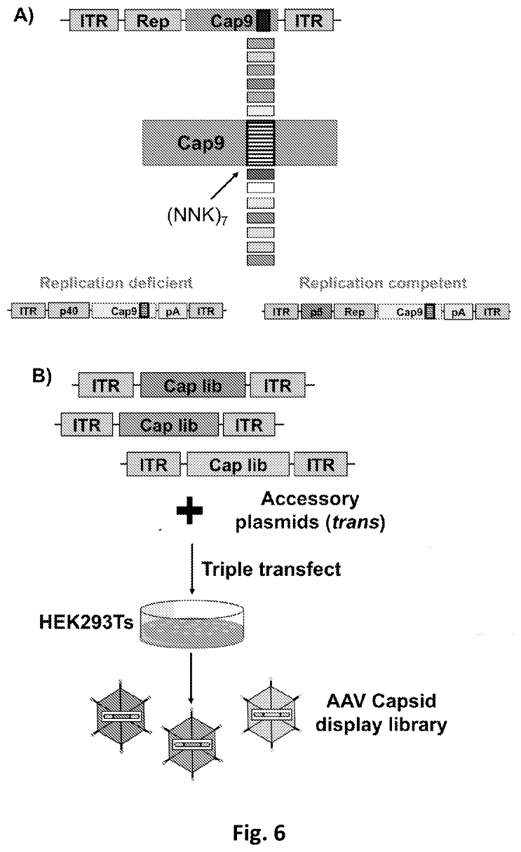

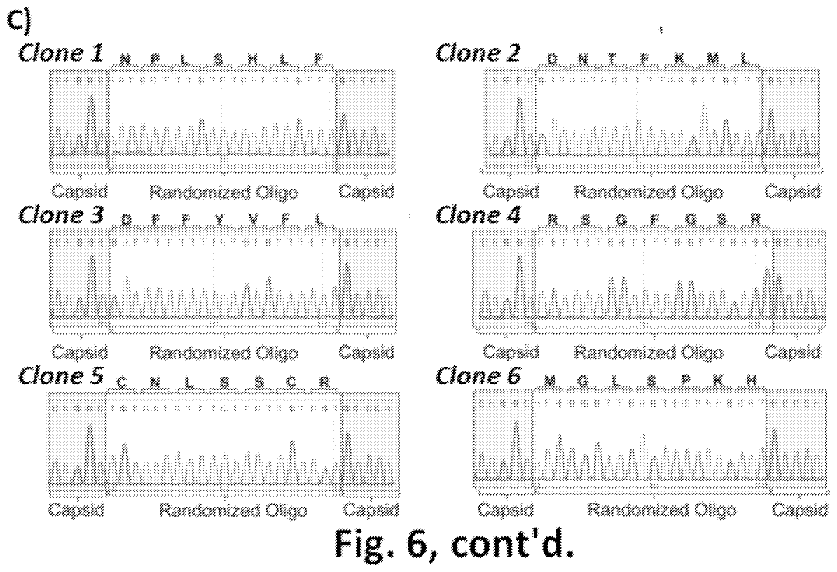

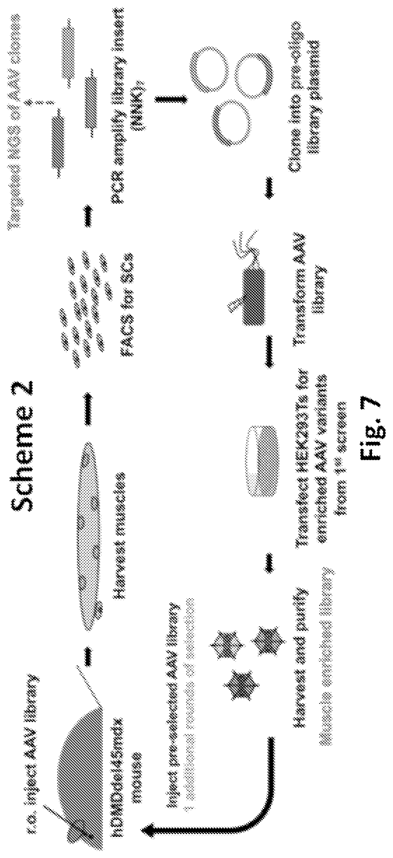

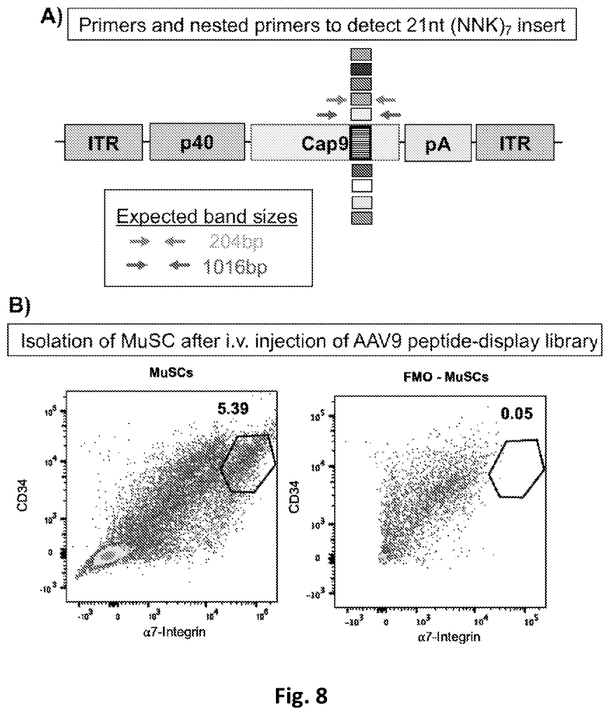

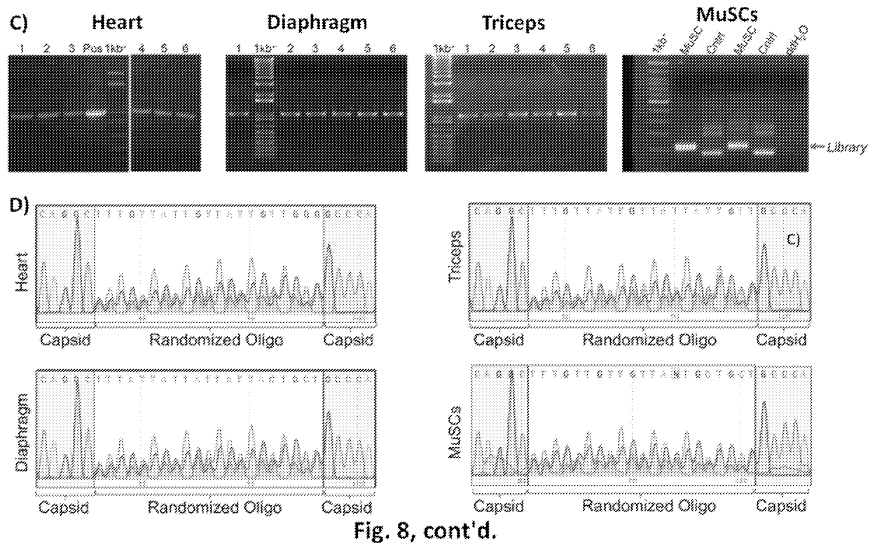

We hypothesized that novel AAV serotypes with a tropism for muscle stem cells (MuSc) could be identified (see, e.g., FIG. 5, Scheme 1. As illustrated in FIG. 6, to improve MuSC targeting strategies, we developed and screened an AAV library to identify new AAV serotypes with the ability to transduce MuSCs. Randomized 21 nucleotide oligo sequences were inserted into the variable surface region of the capsid sequence, which encode a 7-mer peptide displayed on the surface of the viral capsid. Adult mice were injected with the library, and tissues were screened by PCR for the library insert, e.g., as illustrated in Scheme 2 in FIG. 7. We identified unique AAV variants targeting MuSCs as well as the heart and differentiated skeletal muscle (see, e.g., FIG. 8, and Tables 1, 2, and 3, supra.). Identification of MuSC-tropic AAVs can provide a long-lasting supply of CRISPR-corrected MuSCs and offer a sustainable therapy for DMD. It will be recognized that the AAV vectors described herein can be used to deliver CRISPR/cas constructs, and/or base editors, and/or genes for the treatment of numerous diseases that impact skeletal muscle or heart.

### Conclusions.

Recombinant adeno-associated viruses (AAVs) are powerful tools and have become the most commonly used vectors for in vivo gene therapy applications. In particular, the efficacy of AAV is determined by its transduction efficiency in delivering cargo and by its cell type specificity. Recent reports have suggested that AAV9 can efficiently transduce MuSCs; however, we detect very little MuSC targeting and transduction in neonatal mice and virtually no targeting in adults. In order to identify novel serotypes of AAV with re-directed targeting to MuSC or enhanced tissue tropism to cardiac and skeletal muscles, we developed and screened an AAV9 peptide-display library. A randomized 21 oligonucleotide containing an (NNK)₇ (SEQ ID NO:277) triplet design was inserted into a specific locus in the capsid reading frame to display the encoded 7-mer peptide on the surfaces of the viral capsid. Adult mice were injected with the library and tissues were harvested and AAV variants were screened by PCR for the library insert. In addition, we find unique AAV variants targeting MuSCs as well as the heart and differentiated skeletal muscle. A second round of screening using tissue-enriched AAV variants followed by next generation sequencing identified the candidates listed in this application for heart, differentiated skeletal muscle and muscle stem cells. We prioritized lead candidates that demonstrate liver de-targeting, which will allow us to utilize lower vector doses for gene therapy. AAV selectivity to MuSC can be determined by FACS and quantification of vector genomes per diploid genome. Biodistribution and liver de-targeting can be assessed using bioluminescence imaging for luciferase activity. Finally, we can test delivery of our CRISPR/Cas9 gene-editing platform to demonstrate improved efficacy by MuSC AAV serotypes compared to the commonly used AAV9. Identification of MuSC-tropic AAVs may provide a long-lasting supply of CRISPR-corrected MuSCs, which offers a sustainable therapy for DMD.

### REFERENCES

1. Young C S, Hicks M R, Ermolova N V, et al. A Single CRISPR-Cas9 Deletion Strategy that Targets the Majority of DMD Patients Restores Dystrophin Function in hiPSC-Derived Muscle Cells. Cell Stem Cell. 2016; 18(4):533-540.
2. Young C S, Mokhonova E, Quinonez M, Pyle A D, Spencer M J. Creation of a Novel Humanized Dystrophic Mouse Model of Duchenne Muscular Dystrophy and Application of a CRISPR/Cas9 Gene Editing Therapy. J Neuromuscul Dis. 2017; 4(2):139-145.
3. Arnett A L, Konieczny P, Ramos J N, et al. Adeno-associated viral (AAV) vectors do not efficiently target muscle satellite cells. Mol Ther Methods Clin Dev. 2014; 1
4. Varadi K, Michelfelder S, Korff T, et al. Novel random peptide libraries displayed on AAV serotype 9 for selection of endothelial cell-directed gene transfer vectors. Gene Ther. 2012; 19(8):800-8009.
5. Zheng G X, Terry J M, Belgrader P, et al. Massively parallel digital transcriptional profiling of single cells. Nat Commun. 2017; 8:14049.
6. Korbelin J, Trepel M. How to Successfully Screen Random Adeno-Associated Virus Display Peptide Libraries In vivo. Hum Gene Ther Methods. 2017; 28(3):109-123.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. An adeno-associated virus (AAV) vector pseudotyped to transfect muscle and/or muscle stem cells, wherein said vector comprises a capsid protein comprising a peptide insert that improves the ability of said vector to transduce a muscle cell and/or a muscle stem cell.

2. The AAV vector of claim 1, wherein said peptide insert ranges in length from about 7 up to about 15 amino acids.

3. The AAV vector of claim 2, wherein said peptide insert ranges in length from about 7 up to about 10 amino acids, or up to about 9 amino acids.

4. The AAV vector of claim 3, wherein said peptide insert has a length of seven amino acids.

5. The AAV vector according to any one of claims 1-4, wherein said peptide insert comprises or consists of one or more amino acid sequences independently selected from the group consisting of GGMRRDP (SEQ ID NO:1), GVAISGF (SEQ ID NO:2), GQKWNVD (SEQ ID NO:3), GKVGVWA (SEQ ID NO:4), GAFELHN (SEQ ID NO:5), QVLRPTY (SEQ ID NO:6), LGRVHSL (SEQ ID NO:7), LYANVSP (SEQ ID NO:8), LSLGPTV (SEQ ID NO:9), LSLSLAT (SEQ ID NO:10), LNSSSYG (SEQ ID NO: 11), LLRNSTA (SEQ ID NO:12), FHSSASA (SEQ ID NO:13), FVGDTVA (SEQ ID NO:14), FGRGSSG (SEQ ID NO:15), WDKAATF (SEQ ID NO:16), AEAPILL (SEQ ID NO:17), AFHGLLA (SEQ ID NO:18), AGYSIVH (SEQ ID NO:19), ATWNKSQ (SEQ ID NO:20), SVHSDYR (SEQ ID NO:21), SGRNSDL (SEQ ID NO:22), SVFVPMR (SEQ ID NO:23), VHGQYVE (SEQ ID NO:24), VERGASV (SEQ ID NO:25), VSEMKER (SEQ ID NO:26), INHNSHI (SEQ ID NO:27), GQSNISR (SEQ ID NO:28), RQTNQIE (SEQ ID NO:29), GPVVQGA (SEQ ID NO:30), VSGSSFG (SEQ ID NO:31), DVSLTRM (SEQ ID NO:32), NGQGILK (SEQ ID NO:33), SGDAYRA (SEQ ID NO:34), FNNGIHS (SEQ ID NO:35), SSYSVSL (SEQ ID NO:36), ESGNIRP (SEQ ID NO:37), VSGGQTQ (SEQ ID NO:38), IVGSGSA (SEQ ID NO:39), VSSQGTS (SEQ ID NO:40), GSTYENS (SEQ ID NO:41), NGSTSPV (SEQ ID NO:42), EGKSSPT (SEQ ID NO:43), VGQGISL (SEQ ID NO:44), AGIGQAG (SEQ ID NO:45), ITSPSVS (SEQ ID NO:46), EHVEGRR (SEQ ID NO:47), LDGVSHM (SEQ ID NO:48), GGGTLHH (SEQ ID NO:49), HESRPPY (SEQ ID NO:50), SNHDSRV (SEQ ID NO:51), SGSGSFM (SEQ ID NO:52), VGSLYQP (SEQ ID NO:53), GQDNRAL (SEQ ID NO:54), RSEYNSV (SEQ ID NO:55), TGQTVLG (SEQ ID NO:56), GGDAARY (SEQ ID NO:57), RGSNDLP (SEQ ID NO:58), VASGTVL (SEQ ID NO:59), GDHNTIK (SEQ ID NO:60), NARDSGF (SEQ ID NO:61), PGNINNN (SEQ ID NO:62), SCGDSCR (SEQ ID NO:63), FGSVHSP (SEQ ID NO:64), SLGGLGN (SEQ ID NO:65), AADAVRV (SEQ ID NO:66), VIAQGMS (SEQ ID NO:67), NHSSGAM (SEQ ID NO:68), LRDNSAF (SEQ ID NO:69), LGSGALA (SEQ ID NO:70), SHEVVRA (SEQ ID NO:71), FNISANE (SEQ ID NO:72), ARSQEVP (SEQ ID NO:73), AGNITSF (SEQ ID NO:74), TCNSVGC (SEQ ID NO:75), GGGSSSA (SEQ ID NO:76), YDQVSHP (SEQ ID NO:77), VHGISPL (SEQ ID NO:78), TGLNYTT (SEQ ID NO:79), QDRNYGV (SEQ ID NO:80), VSEINRG (SEQ ID NO:81), QRSSNGP (SEQ ID NO:82), SSTGTWV (SEQ ID NO:83), QLPNNLS (SEQ ID NO:84), YNGAIHH (SEQ ID NO:85), MRNTESS (SEQ ID NO:86), GASPSSI (SEQ ID NO:87), DGTYKGA (SEQ ID NO:88), GQGCTQC (SEQ ID NO:89), GAHPTGG (SEQ ID NO:90), SNYNVTG (SEQ ID NO:91), SANVFSP (SEQ ID NO:92), AHSGGTY (SEQ ID NO:93), GENAGRA (SEQ ID NO:94), IGCDYPC (SEQ ID NO:95), ISLPNSI (SEQ ID NO:96), NGINGAS (SEQ ID NO:97), GPSSLRE (SEQ ID NO:98), VQSGNYV (SEQ ID NO:99), GGYSETS (SEQ ID NO:100), LRVGNVE (SEQ ID NO:101), GMGTVAR (SEQ ID NO:102), VQMSGYL (SEQ ID NO:103), QNMVVTS (SEQ ID NO:104), THGLPIV (SEQ ID NO:105), GFNHGAN (SEQ ID NO:106), NSYSVSP (SEQ ID NO:107), SMLGGVA (SEQ ID NO:108), YGVDMGL (SEQ ID NO:109), VIGHGSA (SEQ ID NO:110), SGLDKAP (SEQ ID NO:111), DVKNGGR (SEQ ID NO: 112), IGNGGLI (SEQ ID NO:113), QVGFPTP (SEQ ID NO:114), LSSNANL (SEQ ID NO:115), TNLHVNL (SEQ ID NO: 116), SYIGVAA (SEQ ID NO: 117), STVNGNQ (SEQ ID NO: 118), TNRDAQF (SEQ ID NO:119), LHHSPSY (SEQ ID NO:120), TTYNSSA (SEQ ID NO:121), PSSQPLP (SEQ ID NO:122), RDSMVNP (SEQ ID NO:123), SLHSPQL (SEQ ID NO:124), VQVGTGT (SEQ ID NO:125), RENPSGM (SEQ ID NO:126), GVPKTHD (SEQ ID NO:127), VDQGRLS (SEQ ID NO:128), FDKGAVI (SEQ ID NO:129), VSRSGGV (SEQ ID NO:130), INMQSMA (SEQ ID NO:131), GTASSRP (SEQ ID NO:132), EVGHIRG (SEQ ID NO:133), RDVSSGG (SEQ ID NO:134), ITLSGSS (SEQ ID NO:135), MNGLRGT (SEQ ID NO:136), RSQEINL (SEQ ID NO:137), YGTVSVP (SEQ ID NO:138), LGSSGVS (SEQ ID NO:139), PGSYVSS (SEQ ID NO:140), ASNLGTY (SEQ ID NO:141), GSEGGSM (SEQ ID NO:142), IASGNSV (SEQ ID NO:143), ATAGAII (SEQ ID NO:144), LSSMGQL (SEQ ID NO:145), GEKFMGV (SEQ ID NO:146), GTPSHYG (SEQ ID NO:147), ILSKGAL (SEQ ID NO:148), GMGTVAR (SEQ ID NO:149), RVGHFSA (SEQ ID NO:150), PSMGFTP (SEQ ID NO:151), ALMTGKL (SEQ ID NO:152), PSMTRQT (SEQ ID NO:153), VERGASV (SEQ ID NO:154), KLIGRSP (SEQ ID NO:155), AEAPILL (SEQ ID NO:156), NPNLSLG (SEQ ID NO:157), TMKMFAS (SEQ ID NO:158), SAYNRAA (SEQ ID NO:159), IASTVYG (SEQ ID NO:160), VDSSRVR (SEQ ID NO:161), MNGLRGT (SEQ ID NO:162), YGNSKVV (SEQ ID NO:163), SAVYTRA (SEQ ID NO:164), LAITVAT (SEQ ID NO:165), FVKANVL (SEQ ID NO:166), GEDIGEH (SEQ ID NO:167), KSQLSYV (SEQ ID NO:168), ITLSGSS (SEQ ID NO:169), AKFPVKV (SEQ ID NO:170), SLTAMRG (SEQ ID NO:171), RSEYVRV (SEQ ID NO:172), WNGELVA (SEQ ID NO:173), GQSNISR (SEQ ID NO:174), AEAPILL (SEQ ID NO:175), YGTVSVP (SEQ ID NO:176), GLLNSSP (SEQ ID NO:177), YESSRTA (SEQ ID NO:178), ESGNIRP (SEQ ID NO:179), LNSSSYG (SEQ ID NO:180), DNRTVNV (SEQ ID NO:181), SGSGSFM (SEQ ID NO:182), TRLNTVQ (SEQ ID NO:183), VSGGQTQ (SEQ ID NO:184), DGTYKGA (SEQ ID NO:185), RQTNQIE (SEQ ID NO:186), GTASSRP (SEQ ID NO:187), LGSSGVS (SEQ ID NO:188), AHSGGTY (SEQ ID NO:189), SHEVVRA (SEQ ID NO:190), YISESRP (SEQ ID NO:191), SLGGLGN (SEQ ID NO:192), NSYSVSP (SEQ ID NO:193), GSITYTP (SEQ ID NO:194), NGINGAS (SEQ ID NO:195), VSGSSFG (SEQ ID NO:196), EVRVANA (SEQ ID NO:197), SGRNSDL (SEQ ID NO:198), MTGLRGT (SEQ ID NO:199), GFNHGAN (SEQ ID NO:200), GENAGRA (SEQ ID NO:201), QNMVVTS (SEQ ID NO:202), GASPSSI (SEQ ID NO:203), YVNKSQA (SEQ ID NO:204), GTPSQYG (SEQ ID NO:205), NHSSGAM (SEQ ID NO:206), TNLNLRE (SEQ ID NO:207), GRISGGS (SEQ ID NO:208), VRDLTRV (SEQ ID NO:209), GIGHLAS (SEQ ID NO:210), HESRPPY (SEQ ID NO:211), SSTQKVS (SEQ ID NO:212), FGVSRST (SEQ ID NO:213), MIRGIPS (SEQ ID NO:214), IRQPTSH (SEQ ID NO:215), VSGAVLG (SEQ ID NO:216), RNSDNLS (SEQ ID NO:217), RLASFNS (SEQ ID NO:218), YRIGEGT (SEQ ID NO:219), VHSLGHM (SEQ ID NO:220), VTAVAKV (SEQ ID NO:221), LHHSPSY (SEQ ID NO:222), ERARSTN (SEQ ID NO:223), GMGLTVA (SEQ ID NO:224), LNSSSYG (SEQ ID NO:225), GYKQVSN (SEQ ID NO:226), GKASKDT (SEQ ID NO:227), QLSSRGA (SEQ ID NO:228), VGTRFTS (SEQ ID NO:229), QRSSNGP (SEQ ID NO:230), YKTQGLA (SEQ ID NO:231), RDSMVNP (SEQ ID NO:232), RVSSNLS (SEQ ID NO:233), DGTYKGA (SEQ ID NO:234), FGSGALA (SEQ ID NO:235), ARSQEVP (SEQ ID NO:236), VVVVGRP (SEQ ID NO:237), LHGARSS (SEQ ID NO:238), SGQMRGA (SEQ ID NO:239), TRLNTVQ (SEQ ID NO:240), HMHVCVP (SEQ ID NO:241), VTNSSRV (SEQ ID NO:242), NQLPHFK (SEQ ID NO:243), VRFGSNP (SEQ ID NO:244), YISESRP (SEQ ID NO:245), VSRSGGV (SEQ ID NO:246), LSSNANL (SEQ ID NO:247), DNRTVNV (SEQ ID NO:248), LRVGNVE (SEQ ID NO:249), GTASSRP (SEQ ID NO:250), LHGARYS (SEQ ID NO:251), TGVNVKS (SEQ ID NO:252), VTSWTKN (SEQ ID NO:253), RGSTMMP (SEQ ID NO:254), SGSGSFM (SEQ ID NO:255), VSGGQTQ (SEQ ID NO:256), GMGTVAR (SEQ ID NO:257), LNPRSSS (SEQ ID NO:258), SQNLLSN (SEQ ID NO:259), YRIGEGT (SEQ ID NO:260), STVNGNQ (SEQ ID NO:261), AADAVRV (SEQ ID NO:262), YGNSKVV (SEQ ID NO:263), VPGEGRV (SEQ ID NO:264), GGDAARY (SEQ ID NO:265), YDQVSHP (SEQ ID NO:266), GSFHGTV (SEQ ID NO:267), GAHPTGG (SEQ ID NO:268), RDGQSRG (SEQ ID NO:269), SLGGLGN (SEQ ID NO:270), DGTYKGA (SEQ ID NO:271), GIGHLAS (SEQ ID NO:272), IGSVGKL (SEQ ID NO:273), SHEVVRA (SEQ ID NO:274), EVGHIRG (SEQ ID NO:275), and GLRGVSV (SEQ ID NO:276).

6. The AAV vector of claim 5, wherein said peptide insert comprises or consists of an amino acid sequence selected from the group consisting of GGMRRDP (SEQ ID NO:1), GVAISGF (SEQ ID NO:2), GQKWNVD (SEQ ID NO:3), GKVGVWA (SEQ ID NO:4), GAFELHN (SEQ ID NO:5), QVLRPTY (SEQ ID NO:6), LGRVHSL (SEQ ID NO:7), LYANVSP (SEQ ID NO:8), LSLGPTV (SEQ ID NO:9), LSLSLAT (SEQ ID NO:10), LNSSSYG (SEQ ID NO: 11), LLRNSTA (SEQ ID NO:12), FHSSASA (SEQ ID NO:13), FVGDTVA (SEQ ID NO:14), FGRGSSG (SEQ ID NO:15), WDKAATF (SEQ ID NO:16), AEAPILL (SEQ ID NO:17), AFHGLLA (SEQ ID NO:18), AGYSIVH (SEQ ID NO:19), ATWNKSQ (SEQ ID NO:20), SVHSDYR (SEQ ID NO:21), SGRNSDL (SEQ ID NO:22), SVFVPMR (SEQ ID NO:23), VHGQYVE (SEQ ID NO:24), VERGASV (SEQ ID NO:25), VSEMKER (SEQ ID NO:26), GTPSHYG (SEQ ID NO:27), ILSKGAL (SEQ ID NO:28), GMGTVAR (SEQ ID NO:29), RVGHFSA (SEQ ID NO:30), PSMGFTP (SEQ ID NO:31), ALMTGKL (SEQ ID NO:32), PSMTRQT (SEQ ID NO:33), VERGASV (SEQ ID NO:34), KLIGRSP (SEQ ID NO:35), AEAPILL (SEQ ID NO:36), NPNLSLG (SEQ ID NO:37), TMKMFAS (SEQ ID NO:38), SAYNRAA (SEQ ID NO:39), IASTVYG (SEQ ID NO:40), VDSSRVR (SEQ ID NO:41), MNGLRGT (SEQ ID NO:42), YGNSKVV (SEQ ID NO:43), SAVYTRA (SEQ ID NO:44), LAITVAT (SEQ ID NO:45), FVKANVL (SEQ ID NO:46), GEDIGEH (SEQ ID NO:47), KSQLSYV (SEQ ID NO:48), ITLSGSS (SEQ ID NO:49), AKFPVKV (SEQ ID NO:50), SLTAMRG (SEQ ID NO:51), RSEYVRV (SEQ ID NO:52), VRDLTRV (SEQ ID NO:53), GIGHLAS (SEQ ID NO:54), HESRPPY (SEQ ID NO:55), SSTQKVS (SEQ ID NO:56), FGVSRST (SEQ ID NO:57), MIRGIPS (SEQ ID NO:58), IRQPTSH (SEQ ID NO:59), VSGAVLG (SEQ ID NO:60), RNSDNLS (SEQ ID NO:61), RLASFNS (SEQ ID NO:62), YRIGEGT (SEQ ID NO:63), VHSLGHM (SEQ ID NO:64), VTAVAKV (SEQ ID NO:65), LHHSPSY (SEQ ID NO:66), and ERARSTN (SEQ ID NO:67).

7. The AAV vector of claim 5, wherein said vector transduces heart muscle and said peptide insert comprises or consists of one or more amino acid sequences selected from the group consisting of GGMRRDP (SEQ ID NO:1), GVAISGF (SEQ ID NO:2), GQKWNVD (SEQ ID NO:3), GKVGVWA (SEQ ID NO:4), GAFELHN (SEQ ID NO:5), QVLRPTY (SEQ ID NO:6), LGRVHSL (SEQ ID NO:7), LYANVSP (SEQ ID NO:8), LSLGPTV (SEQ ID NO:9), LSLSLAT (SEQ ID NO:10), LNSSSYG (SEQ ID NO:11), LLRNSTA (SEQ ID NO:12), FHSSASA (SEQ ID NO:13), FVGDTVA (SEQ ID NO:14), FGRGSSG (SEQ ID NO:15), WDKAATF (SEQ ID NO:16), AEAPILL (SEQ ID NO:17), AFHGLLA (SEQ ID NO:18), AGYSIVH (SEQ ID NO:19), ATWNKSQ (SEQ ID NO:20), SVHSDYR (SEQ ID NO:21), SGRNSDL (SEQ ID NO:22), SVFVPMR (SEQ ID NO:23), VHGQYVE (SEQ ID NO:24), VERGASV (SEQ ID NO:25), VSEMKER (SEQ ID NO:26), INHNSHI (SEQ ID NO:27), GQSNISR (SEQ ID NO:28), RQTNQIE (SEQ ID NO:29), GPVVQGA (SEQ ID NO:30), VSGSSFG (SEQ ID NO:31), DVSLTRM (SEQ ID NO:32), NGQGILK (SEQ ID NO:33), SGDAYRA (SEQ ID NO:34), FNNGIHS (SEQ ID NO:35), SSYSVSL (SEQ ID NO:36), ESGNIRP (SEQ ID NO:37), VSGGQTQ (SEQ ID NO:38), IVGSGSA (SEQ ID NO:39), VSSQGTS (SEQ ID NO:40), GSTYENS (SEQ ID NO:41), NGSTSPV (SEQ ID NO:42), EGKSSPT (SEQ ID NO:43), VGQGISL (SEQ ID NO:44), AGIGQAG (SEQ ID NO:45), ITSPSVS (SEQ ID NO:46), EHVEGRR (SEQ ID NO:47), LDGVSHM (SEQ ID NO:48), GGGTLHH (SEQ ID NO:49), HESRPPY (SEQ ID NO:50), SNHDSRV (SEQ ID NO:51), SGSGSFM (SEQ ID NO:52), VGSLYQP (SEQ ID NO:53), GQDNRAL (SEQ ID NO:54), RSEYNSV (SEQ ID NO:55), TGQTVLG (SEQ ID NO:56), GGDAARY (SEQ ID NO:57), RGSNDLP (SEQ ID NO:58), VASGTVL (SEQ ID NO:59), GDHNTIK (SEQ ID NO:60), NARDSGF (SEQ ID NO:61), PGNINNN (SEQ ID NO:62), SCGDSCR (SEQ ID NO:63), FGSVHSP (SEQ ID NO:64), SLGGLGN (SEQ ID NO:65), AADAVRV (SEQ ID NO:66), VIAQGMS (SEQ ID NO:67), NHSSGAM (SEQ ID NO:68), LRDNSAF (SEQ ID NO:69), LGSGALA (SEQ ID NO:70), SHEVVRA (SEQ ID NO:71), FNISANE (SEQ ID NO:72), ARSQEVP (SEQ ID NO:73), AGNITSF (SEQ ID NO:74), TCNSVGC (SEQ ID NO:75), GGGSSSA (SEQ ID NO:76), YDQVSHP (SEQ ID NO:77), VHGISPL (SEQ ID NO:78), TGLNYTT (SEQ ID NO:79), QDRNYGV (SEQ ID NO:80), VSEINRG (SEQ ID NO:81), QRSSNGP (SEQ ID NO:82), SSTGTWV (SEQ ID NO:83), QLPNNLS (SEQ ID NO:84), YNGAIHH (SEQ ID NO:85), MRNTESS (SEQ ID NO:86), GASPSSI (SEQ ID NO:87), DGTYKGA (SEQ ID NO:88), GQGCTQC (SEQ ID NO:89), GAHPTGG (SEQ ID NO:90), SNYNVTG (SEQ ID NO:91), SANVFSP (SEQ ID NO:92), AHSGGTY (SEQ ID NO:93), GENAGRA (SEQ ID NO:94), IGCDYPC (SEQ ID NO:95), ISLPNSI (SEQ ID NO:96), NGINGAS (SEQ ID NO:97), GPSSLRE (SEQ ID NO:98), VQSGNYV (SEQ ID NO:99), GGYSETS (SEQ ID NO:100), LRVGNVE (SEQ ID NO:101), GMGTVAR (SEQ ID NO:102), VQMSGYL (SEQ ID NO:103), QNMVVTS (SEQ ID NO:104), THGLPIV (SEQ ID NO:105), GFNHGAN (SEQ ID NO:106), NSYSVSP (SEQ ID NO:107), SMLGGVA (SEQ ID NO:108), YGVDMGL (SEQ ID NO:109), VIGHGSA (SEQ ID NO:110), SGLDKAP (SEQ ID NO:111), DVKNGGR (SEQ ID NO: 112), IGNGGLI (SEQ ID NO:113), QVGFPTP (SEQ ID NO:114), LSSNANL (SEQ ID NO:115), TNLHVNL (SEQ ID NO: 116), SYIGVAA (SEQ ID NO: 117), STVNGNQ (SEQ ID NO: 118), TNRDAQF (SEQ ID NO:119), LHHSPSY (SEQ ID NO:120), TTYNSSA (SEQ ID NO:121), PSSQPLP (SEQ ID NO:122), RDSMVNP (SEQ ID NO:123), SLHSPQL (SEQ ID NO:124), VQVGTGT (SEQ ID NO:125), RENPSGM (SEQ ID NO:126), GVPKTHD (SEQ ID NO:127), VDQGRLS (SEQ ID NO:128), FDKGAVI (SEQ ID NO:129), VSRSGGV (SEQ ID NO:130), INMQSMA (SEQ ID NO:131), GTASSRP (SEQ ID NO:132), EVGHIRG (SEQ ID NO:133), RDVSSGG (SEQ ID NO:134), ITLSGSS (SEQ ID NO:135), MNGLRGT (SEQ ID NO:136), RSQEINL (SEQ ID NO:137), YGTVSVP (SEQ ID NO:138), LGSSGVS (SEQ ID NO:139), PGSYVSS (SEQ ID NO:140), ASNLGTY (SEQ ID NO:141), GSEGGSM (SEQ ID NO:142), IASGNSV (SEQ ID NO:143), ATAGAII (SEQ ID NO:144), LSSMGQL (SEQ ID NO:145), and GEKFMGV (SEQ ID NO:146).

8. The AAV vector of claim 7, wherein said vector transduces heart muscle and said peptide insert comprises or consists of one or more amino acid sequences selected from the group consisting of GGMRRDP (SEQ ID NO:1), GVAISGF (SEQ ID NO:2), GQKWNVD (SEQ ID NO:3), GKVGVWA (SEQ ID NO:4), GAFELHN (SEQ ID NO:5), QVLRPTY (SEQ ID NO:6), LGRVHSL (SEQ ID NO:7), LYANVSP (SEQ ID NO:8), LSLGPTV (SEQ ID NO:9), LSLSLAT (SEQ ID NO:10), LNSSSYG (SEQ ID NO: 11), LLRNSTA (SEQ ID NO:12), FHSSASA (SEQ ID NO:13), FVGDTVA (SEQ ID NO:14), FGRGSSG (SEQ ID NO:15), WDKAATF (SEQ ID NO:16), AEAPILL (SEQ ID NO:17), AFHGLLA (SEQ ID NO:18), AGYSIVH (SEQ ID NO:19), ATWNKSQ (SEQ ID NO:20), SVHSDYR (SEQ ID NO:21), SGRNSDL (SEQ ID NO:22), SVFVPMR (SEQ ID NO:23), VHGQYVE (SEQ ID NO:24), VERGASV (SEQ ID NO:25), and VSEMKER (SEQ ID NO:26).

9. The AAV vector of claim 7, wherein said vector transduces heart muscle and said peptide insert comprises or consists of one or more amino acid sequences selected from the group consisting of INHNSHI (SEQ ID NO:27), GQSNISR (SEQ ID NO:28), RQTNQIE (SEQ ID NO:29), GPVVQGA (SEQ ID NO:30), VSGSSFG (SEQ ID NO:31), DVSLTRM (SEQ ID NO:32), NGQGILK (SEQ ID NO:33), SGDAYRA (SEQ ID NO:34), FNNGIHS (SEQ ID NO:35), SSYSVSL (SEQ ID NO:36), ESGNIRP (SEQ ID NO:37), VSGGQTQ (SEQ ID NO:38), IVGSGSA (SEQ ID NO:39), VSSQGTS (SEQ ID NO:40), GSTYENS (SEQ ID NO:41), NGSTSPV (SEQ ID NO:42), EGKSSPT (SEQ ID NO:43), VGQGISL (SEQ ID NO:44), AGIGQAG (SEQ ID NO:45), ITSPSVS (SEQ ID NO:46), EHVEGRR (SEQ ID NO:47), LDGVSHM (SEQ ID NO:48), GGGTLHH (SEQ ID NO:49), HESRPPY (SEQ ID NO:50), SNHDSRV (SEQ ID NO:51), SGSGSFM (SEQ ID NO:52), VGSLYQP (SEQ ID NO:53), GQDNRAL (SEQ ID NO:54), RSEYNSV (SEQ ID NO:55), TGQTVLG (SEQ ID NO:56), GGDAARY (SEQ ID NO:57), RGSNDLP (SEQ ID NO:58), VASGTVL (SEQ ID NO:59), GDHNTIK (SEQ ID NO:60), NARDSGF (SEQ ID NO:61), PGNINNN (SEQ ID NO:62), SCGDSCR (SEQ ID NO:63), FGSVHSP (SEQ ID NO:64), SLGGLGN (SEQ ID NO:65), AADAVRV (SEQ ID NO:66), VIAQGMS (SEQ ID NO:67), NHSSGAM (SEQ ID NO:68), LRDNSAF (SEQ ID NO:69), LGSGALA (SEQ ID NO:70), SHEVVRA (SEQ ID NO:71), FNISANE (SEQ ID NO:72), ARSQEVP (SEQ ID NO:73), AGNITSF (SEQ ID NO:74), TCNSVGC (SEQ ID NO:75), GGGSSSA (SEQ ID NO:76), YDQVSHP (SEQ ID NO:77), VHGISPL (SEQ ID NO:78), TGLNYTT (SEQ ID NO:79), QDRNYGV (SEQ ID NO:80), VSEINRG (SEQ ID NO:81), QRSSNGP (SEQ ID NO:82), SSTGTWV (SEQ ID NO:83), QLPNNLS (SEQ ID NO:84), YNGAIHH (SEQ ID NO:85), MRNTESS (SEQ ID NO:86), GASPSSI (SEQ ID NO:87), DGTYKGA (SEQ ID NO:88), GQGCTQC (SEQ ID NO:89), GAHPTGG (SEQ ID NO:90), SNYNVTG (SEQ ID NO:91), SANVFSP (SEQ ID NO:92), AHSGGTY (SEQ ID NO:93), GENAGRA (SEQ ID NO:94), IGCDYPC (SEQ ID NO:95), ISLPNSI (SEQ ID NO:96), NGINGAS (SEQ ID NO:97), GPSSLRE (SEQ ID NO:98), VQSGNYV (SEQ ID NO:99), GGYSETS (SEQ ID NO:100), LRVGNVE (SEQ ID NO:101), GMGTVAR (SEQ ID NO:102), VQMSGYL (SEQ ID NO:103), QNMVVTS (SEQ ID NO:104), THGLPIV (SEQ ID NO:105), GFNHGAN (SEQ ID NO:106), NSYSVSP (SEQ ID NO:107), SMLGGVA (SEQ ID NO:108), YGVDMGL (SEQ ID NO:109), VIGHGSA (SEQ ID NO: 110), SGLDKAP (SEQ ID NO:111), DVKNGGR (SEQ ID NO:112), IGNGGLI (SEQ ID NO: 113), QVGFPTP (SEQ ID NO: 114), LSSNANL (SEQ ID NO:115), TNLHVNL (SEQ ID NO: 116), SYIGVAA (SEQ ID NO:117), STVNGNQ (SEQ ID NO:118), TNRDAQF (SEQ ID NO:119), LHHSPSY (SEQ ID NO:120), TTYNSSA (SEQ ID NO:121), PSSQPLP (SEQ ID NO:122), RDSMVNP (SEQ ID NO:123), SLHSPQL (SEQ ID NO:124), VQVGTGT (SEQ ID NO:125), RENPSGM (SEQ ID NO:126), GVPKTHD (SEQ ID NO:127), VDQGRLS (SEQ ID NO:128), FDKGAVI (SEQ ID NO:129), VSRSGGV (SEQ ID NO:130), INMQSMA (SEQ ID NO:131), GTASSRP (SEQ ID NO:132), EVGHIRG (SEQ ID NO:133), RDVSSGG (SEQ ID NO:134), ITLSGSS (SEQ ID NO:135), MNGLRGT (SEQ ID NO:136), RSQEINL (SEQ ID NO:137), YGTVSVP (SEQ ID NO:138), LGSSGVS (SEQ ID NO:139), PGSYVSS (SEQ ID NO:140), ASNLGTY (SEQ ID NO:141), GSEGGSM (SEQ ID NO:142), IASGNSV (SEQ ID NO:143), ATAGAII (SEQ ID NO:144), LSSMGQL (SEQ ID NO:145), and GEKFMGV (SEQ ID NO:146).

10. The AAV vector of claim 5, wherein said vector transduces skeletal muscle and said peptide insert comprises or consists of one or more amino acid sequences selected from the group consisting of GTPSHYG (SEQ ID NO:147), ILSKGAL (SEQ ID NO:148), GMGTVAR (SEQ ID NO:149), RVGHFSA (SEQ ID NO:150), PSMGFTP (SEQ ID NO:151), ALMTGKL (SEQ ID NO:152), PSMTRQT (SEQ ID NO:153), VERGASV (SEQ ID NO:154), KLIGRSP (SEQ ID NO:155), AEAPILL (SEQ ID NO:156), NPNLSLG (SEQ ID NO:157), TMKMFAS (SEQ ID NO:158), SAYNRAA (SEQ ID NO:159), IASTVYG (SEQ ID NO:160), VDSSRVR (SEQ ID NO:161), MNGLRGT (SEQ ID NO:162), YGNSKVV (SEQ ID NO:163), SAVYTRA (SEQ ID NO:164), LAITVAT (SEQ ID NO:165), FVKANVL (SEQ ID NO:166), GEDIGEH (SEQ ID NO:167), KSQLSYV (SEQ ID NO:168), ITLSGSS (SEQ ID NO:169), AKFPVKV (SEQ ID NO:170), SLTAMRG (SEQ ID NO:171), RSEYVRV (SEQ ID NO:172), WNGELVA (SEQ ID NO:173), GQSNISR (SEQ ID NO:174), AEAPILL (SEQ ID NO:175), YGTVSVP (SEQ ID NO:176), GLLNSSP (SEQ ID NO:177), YESSRTA (SEQ ID NO:178), ESGNIRP (SEQ ID NO:179), LNSSSYG (SEQ ID NO:180), DNRTVNV (SEQ ID NO:181), SGSGSFM (SEQ ID NO:182), TRLNTVQ (SEQ ID NO:183), VSGGQTQ (SEQ ID NO:184), DGTYKGA (SEQ ID NO:185), RQTNQIE (SEQ ID NO:186), GTASSRP (SEQ ID NO:187), LGSSGVS (SEQ ID NO:188), AHSGGTY (SEQ ID NO:189), SHEVVRA (SEQ ID NO:190), YISESRP (SEQ ID NO:191), SLGGLGN (SEQ ID NO:192), NSYSVSP (SEQ ID NO:193), GSITYTP (SEQ ID NO:194), NGINGAS (SEQ ID NO:195), VSGSSFG (SEQ ID NO:196), EVRVANA (SEQ ID NO:197), SGRNSDL (SEQ ID NO:198), MTGLRGT (SEQ ID NO:199), GFNHGAN (SEQ ID NO:200), GENAGRA (SEQ ID NO:201), QNMVVTS (SEQ ID NO:202), GASPSSI (SEQ ID NO:203), YVNKSQA (SEQ ID NO:204), GTPSQYG (SEQ ID NO:205), NHSSGAM (SEQ ID NO:206), TNLNLRE (SEQ ID NO:207), and GRISGGS (SEQ ID NO:208).

11. The AAV vector of claim 10, wherein said vector transduces skeletal muscle and said peptide insert comprises or consists of one or more amino acid sequences selected from the group consisting of GTPSHYG (SEQ ID NO:147), ILSKGAL (SEQ ID NO:148), GMGTVAR (SEQ ID NO:149), RVGHFSA (SEQ ID NO:150), PSMGFTP (SEQ ID NO:151), ALMTGKL (SEQ ID NO:152), PSMTRQT (SEQ ID NO:153), VERGASV (SEQ ID NO:154), KLIGRSP (SEQ ID NO:155), AEAPILL (SEQ ID NO:156), NPNLSLG (SEQ ID NO:157), TMKMFAS (SEQ ID NO:158), SAYNRAA (SEQ ID NO:159), IASTVYG (SEQ ID NO:160), VDSSRVR (SEQ ID NO:161), MNGLRGT (SEQ ID NO:162), YGNSKVV (SEQ ID NO:163), SAVYTRA (SEQ ID NO:164), LAITVAT (SEQ ID NO:165), FVKANVL (SEQ ID NO:166), GEDIGEH (SEQ ID NO:167), KSQLSYV (SEQ ID NO:168), ITLSGSS (SEQ ID NO:169), AKFPVKV (SEQ ID NO:170), SLTAMRG (SEQ ID NO:171), and RSEYVRV (SEQ ID NO:172).

12. The AAV vector of claim 10, wherein said vector transduces skeletal muscle and said peptide insert comprises or consists of one or more amino acid sequences selected from the group consisting of WNGELVA (SEQ ID NO:173), GQSNISR (SEQ ID NO:174), AEAPILL (SEQ ID NO:175), YGTVSVP (SEQ ID NO:176), GLLNSSP (SEQ ID NO:177), YESSRTA (SEQ ID NO:178), ESGNIRP (SEQ ID NO:179), LNSSSYG (SEQ ID NO:180), DNRTVNV (SEQ ID NO:181), SGSGSFM (SEQ ID NO:182), TRLNTVQ (SEQ ID NO:183), VSGGQTQ (SEQ ID NO:184), DGTYKGA (SEQ ID NO:185), RQTNQIE (SEQ ID NO:186), GTASSRP (SEQ ID NO:187), LGSSGVS (SEQ ID NO:188), AHSGGTY (SEQ ID NO:189), SHEVVRA (SEQ ID NO:190), YISESRP (SEQ ID NO:191), SLGGLGN (SEQ ID NO:192), NSYSVSP (SEQ ID NO:193), GSITYTP (SEQ ID NO:194), NGINGAS (SEQ ID NO:195), VSGSSFG (SEQ ID NO:196), EVRVANA (SEQ ID NO:197), SGRNSDL (SEQ ID NO:198), MTGLRGT (SEQ ID NO:199), GFNHGAN (SEQ ID NO:200), GENAGRA (SEQ ID NO:201), QNMVVTS (SEQ ID NO:202), GASPSSI (SEQ ID NO:203), YVNKSQA (SEQ ID NO:204), GTPSQYG (SEQ ID NO:205), NHSSGAM (SEQ ID NO:206), TNLNLRE (SEQ ID NO:207), and GRISGGS (SEQ ID NO:208).

13. The AAV vector of claim 5, wherein said vector transduces muscle stem cells and said peptide insert comprises or consists of an amino acid sequence selected from the group consisting of VRDLTRV (SEQ ID NO:209), GIGHLAS (SEQ ID NO:210), HESRPPY (SEQ ID NO:211), SSTQKVS (SEQ ID NO:212), FGVSRST (SEQ ID NO:213), MIRGIPS (SEQ ID NO:214), IRQPTSH (SEQ ID NO:215), VSGAVLG (SEQ ID NO:216), RNSDNLS (SEQ ID NO:217), RLASFNS (SEQ ID NO:218), YRIGEGT (SEQ ID NO:219), VHSLGHM (SEQ ID NO:220), VTAVAKV (SEQ ID NO:221), LHHSPSY (SEQ ID NO:222), ERARSTN (SEQ ID NO:223), GMGLTVA (SEQ ID NO:224), LNSSSYG (SEQ ID NO:225), GYKQVSN (SEQ ID NO:226), GKASKDT (SEQ ID NO:227), QLSSRGA (SEQ ID NO:228), VGTRFTS (SEQ ID NO:229), QRSSNGP (SEQ ID NO:230), YKTQGLA (SEQ ID NO:231), RDSMVNP (SEQ ID NO:232), RVSSNLS (SEQ ID NO:233), DGTYKGA (SEQ ID NO:234), FGSGALA (SEQ ID NO:235), ARSQEVP (SEQ ID NO:236), VVVVGRP (SEQ ID NO:237), LHGARSS (SEQ ID NO:238), SGQMRGA (SEQ ID NO:239), TRLNTVQ (SEQ ID NO:240), HMHVCVP (SEQ ID NO:241), VTNSSRV (SEQ ID NO:242), NQLPHFK (SEQ ID NO:243), VRFGSNP (SEQ ID NO:244), YISESRP (SEQ ID NO:245), VSRSGGV (SEQ ID NO:246), LSSNANL (SEQ ID NO:247), DNRTVNV (SEQ ID NO:248), LRVGNVE (SEQ ID NO:249), GTASSRP (SEQ ID NO:250), LHGARYS (SEQ ID NO:251), TGVNVKS (SEQ ID NO:252), VTSWTKN (SEQ ID NO:253), RGSTMMP (SEQ ID NO:254), SGSGSFM (SEQ ID NO:255), VSGGQTQ (SEQ ID NO:256), GMGTVAR (SEQ ID NO:257), LNPRSSS (SEQ ID NO:258), SQNLLSN (SEQ ID NO:259), YRIGEGT (SEQ ID NO:260), STVNGNQ (SEQ ID NO:261), AADAVRV (SEQ ID NO:262), YGNSKVV (SEQ ID NO:263), VPGEGRV (SEQ ID NO:264), GGDAARY (SEQ ID NO:265), YDQVSHP (SEQ ID NO:266), GSFHGTV (SEQ ID NO:278), GAHPTGG (SEQ ID NO:279), RDGQSRG (SEQ ID NO:280), SLGGLGN (SEQ ID NO:281), DGTYKGA (SEQ ID NO:282), GIGHLAS (SEQ ID NO:283), IGSVGKL (SEQ ID NO:284), SHEVVRA (SEQ ID NO:285), EVGHIRG (SEQ ID NO:286), and GLRGVSV (SEQ ID NO:287).

14. The AAV vector of claim 13, wherein said vector transduces muscle stem cells and said peptide insert comprises or consists of one or more an amino acid sequences selected from the group consisting of VRDLTRV (SEQ ID NO:209), GIGHLAS (SEQ ID NO:210), HESRPPY (SEQ ID NO:211), SSTQKVS (SEQ ID NO:212), FGVSRST (SEQ ID NO:213), MIRGIPS (SEQ ID NO:214), IRQPTSH (SEQ ID NO:215), VSGAVLG (SEQ ID NO:216), RNSDNLS (SEQ ID NO:217), RLASFNS (SEQ ID NO:218), YRIGEGT (SEQ ID NO:219), VHSLGHM (SEQ ID NO:220), VTAVAKV (SEQ ID NO:221), LHHSPSY (SEQ ID NO:222), ERARSTN (SEQ ID NO:223), GMGLTVA (SEQ ID NO:224), LNSSSYG (SEQ ID NO:225), GYKQVSN (SEQ ID NO:226), GKASKDT (SEQ ID NO:227), QLSSRGA (SEQ ID NO:228), VGTRFTS (SEQ ID NO:229), QRSSNGP (SEQ ID NO:230), YKTQGLA (SEQ ID NO:231), RDSMVNP (SEQ ID NO:232), RVSSNLS (SEQ ID NO:233), and DGTYKGA (SEQ ID NO:234).

15. The AAV vector of claim 13, wherein said vector transduces muscle stem cells and said peptide insert comprises or consists of one or more an amino acid sequences selected from the group consisting of FGSGALA (SEQ ID NO:235), ARSQEVP (SEQ ID NO:236), VVVVGRP (SEQ ID NO:237), LHGARSS (SEQ ID NO:238), SGQMRGA (SEQ ID NO:239), TRLNTVQ (SEQ ID NO:240), HMHVCVP (SEQ ID NO:241), VTNSSRV (SEQ ID NO:242), NQLPHFK (SEQ ID NO:243), VRFGSNP (SEQ ID NO:244), YISESRP (SEQ ID NO:245), VSRSGGV (SEQ ID NO:246), LSSNANL (SEQ ID NO:247), DNRTVNV (SEQ ID NO:248), LRVGNVE (SEQ ID NO:249), GTASSRP (SEQ ID NO:250), LHGARYS (SEQ ID NO:251), TGVNVKS (SEQ ID NO:252), VTSWTKN (SEQ ID NO:253), RGSTMMP (SEQ ID NO:254), SGSGSFM (SEQ ID NO:255), VSGGQTQ (SEQ ID NO:256), GMGTVAR (SEQ ID NO:257), LNPRSSS (SEQ ID NO:258), SQNLLSN (SEQ ID NO:259), YRIGEGT (SEQ ID NO:260), STVNGNQ (SEQ ID NO:261), AADAVRV (SEQ ID NO:262), YGNSKVV (SEQ ID NO:263), VPGEGRV (SEQ ID NO:264), GGDAARY (SEQ ID NO:265), YDQVSHP (SEQ ID NO:266), GSFHGTV (SEQ ID NO:288), GAHPTGG (SEQ ID NO:289), RDGQSRG (SEQ ID NO:290), SLGGLGN (SEQ ID NO:291), DGTYKGA (SEQ ID NO:292), GIGHLAS (SEQ ID NO:293), IGSVGKL (SEQ ID NO:294), SHEVVRA (SEQ ID NO:295), EVGHIRG (SEQ ID NO:296), GLRGVSV (SEQ ID NO:297).

16. The AAV vector according to any one of claims 1-15, wherein said vector comprises a serotype selected from the group consisting of AAV1, AAV2, AAV3, AAV4, AAV5, AAV6, AAV7, AAV8, AAV9, AAV10, and AAVrh74.

17. The AAV vector of claim 16, wherein said vector comprises a serotype selected from the group consisting of AAV6, AAVrh74, and AAV9.

18. The AAV vector of claim 17, wherein said vector comprises an AAV9 serotype.

19. The AAV vector according to any one of claims 1-18, wherein said peptide insert is disposed in viral protein 1 (VP1).

20. The AAV vector of claim 19, wherein said peptide insert is disposed in in variable surface region VIII (VR-VIII) of viral protein 1.

21. The AAV vector of claim 20, wherein said peptide insert is disposed between residues 581-593 of viral protein 1.

22. The AAV vector of claim 21, wherein said peptide insert is disposed between residues 585-592 of viral protein 1.

23. The AAV vector of claim 22, wherein said peptide insert is disposed between residues 589-590 of viral protein 1.

24. The AAV vector according to any one of claims 1-23, wherein said system further comprises a nucleic acid that encodes a construct that is to be delivered to a muscle cell and/or to a muscle stem cell, where said construct is flanked by AAV inverted terminal repeat (ITR) sequences.

25. The AAV vector of claim 24, wherein said construct comprises a gene/cDNA that is to be introduced into said muscle cell and/or muscle stem cell.

26. The AAV vector of claim 25, wherein said gene/cDNA comprises a gene or cDNA selected for the treatment of a disease that impacts skeletal muscle or heart.

27. The AAV vector of claim 24, wherein said construct comprises a nucleic acid that encodes a gene-editing protein.

28. The AAV vector of claim 27, wherein said gene editing protein comprises a component of a CRISPR/Cas system, a CRISPR base editor, a TALEN, and a zinc finger protein.

29. The AAV vector of claim 28, wherein said construct encodes a CRISPR protein and a guide RNA.

30. The AAV vector according to any one of claims 1-29, wherein vector has a reduced tropism for liver as compared to the same vector lacking said inserted peptide.

31. A nucleic acid encoding an adeno-associated virus (AAV) capsid protein comprising a peptide insert that improves the ability of an AAV vector comprising said capsid protein said vector to transduce a muscle cell and/or a muscle stem cell.

32. The nucleic acid of claim 31, wherein said nucleic acid encodes a capsid protein of the AAV vector according to any one of claims 1-23.

33. The nucleic acid according to any one of claims 31-32, wherein said nucleic acid comprises a plasmid.

34. An adeno-associated virus (AAV) packaging system for producing a pseudotyped AAV vector having tropism for muscle cells and/or muscle stem cells said system comprising:
a plasmid that encodes a capsid protein wherein said plasmid comprises a nucleic acid sequence according to any one of claims 31-33; and
a plasmid that comprises AAV helper genes.

35. The packaging system of claim 34, wherein said plasmid that encodes a capsid protein, comprises an AAV Rep gene.

36. The packaging system according to any one of claims 34-35, wherein said plasmid that comprises AAV helper genes encodes AAV genes E4, E2A, and Va.

37. The packaging system according to any one of claims 34-36, wherein said system further comprises a packaging cell.

38. The packaging system of claim 37, wherein said packaging cell comprises a cell selected from the group consisting of HEK293, HEK293T, TE671, HT1080, 3T3, K562, 3T3, U937, and H9.

39. The packaging system of claim 38, wherein said packaging cell comprises an HEK293T cell.

40. The packaging system according to any one of claims 34-39, wherein said system further comprises a nucleic acid that encodes a construct that is to be delivered to a muscle cell and/or to a muscle stem cell, where said construct is flanked by AAV inverted terminal repeat (ITR) sequences.

41. The packaging system of claim 40, wherein said construct comprises a gene/cDNA that is to be introduced into said muscle cell and/or muscle stem cell.

42. The packaging system of claim 41, wherein said gene/cDNA comprises a gene or cDNA selected for the treatment of a skeletal muscle disorder selected from the group consisting of Limb girdle muscular dystrophy (LGMD), myofibrillar myopathy, myotubular myopathy, BODY, inclusion body myopathy, and vacuolar myopathy.

43. The packaging system of claim 42, wherein said skeletal muscle disorder comprises limb girdle muscular dystrophy (LGMD).

44. The packaging system according to any one of claims 42-43, wherein said gene comprise a gene selected from the group consisting of CAPN3, DYSF, TRIM32, ACTA1, MTM1, PAX7, DES, and LAMP2.

45. The packaging system of claim 44, wherein said gene comprises CAPN3.

46. The packaging system of claim 45, wherein said gene comprises a full-length CAPN3 cDNA.

47. The packaging system of claim 45, wherein said gene comprises a UTR-deleted, Kozak-optimized CAPN3 cDNA.

48. The packaging system of claim 40, wherein said construct comprises a nucleic acid that encodes a gene-editing protein.

49. The packaging system of claim 48, wherein said gene editing protein comprises a component of a CRISPR/Cas system, a TALEN, and a zinc finger protein.

50. The packaging system of claim 49, wherein construct encodes a CRISPR protein and a guide RNA.

51. The packaging system of claim 50, wherein said construct encodes an RNA to direct said CRISPR protein to cleave exons 45-55 of the DMD gene.

52. A packaging cell for packaging a pseudotyped AAV vector having tropism for muscle cells and/or muscle stem cells, said packaging cell is a mammalian cell comprising a nucleic acid according to any one of claims 31-33 operably linked to a promoter and capable of expressing said capsid protein.

53. The packaging cell of claim 52, wherein said nucleic acid comprises a plasmid disposed episomally in said cell.

54. The packaging cell of claim 52, wherein said nucleic acid is integrated into the genome of said cell.

55. The packaging cell according to any one of claims 52-54, wherein said cell further comprises all the components required for encapsidation of an adeno-associated virus.

56. The packaging cell according to any one of claims 52-54, wherein said cell further comprises nucleic acid(s) encoding encodes AAV genes E4, E2A, and Va.

57. The packaging cell according to any one of claims 52-56, wherein said mammalian cell is selected from the group consisting of HEK293, HEK293T, TE671, HT1080, 3T3, K562, 3T3, U937, and H9.

58. The packaging cell of claim 57, wherein said cell is a HEK293T cell.

59. A method of producing an AAV vector that has a tropism to a muscle cell and/or to a muscle stem cell, said method comprising:
providing:
an adeno-associated virus (AAV) packaging system according to any one of claims 34-36;
a nucleic acid encoding a construct that is to be delivered into a muscle cell and/or a muscle stem cell where said construct is flanked by ITR sequences; and
a helper cell;
where said helper cells packages said nucleic acid encoding a construct into a pseudotype AAV viral capsid that has a tropism to muscle cells and/or to muscle stem cells.

60. A method of producing an AAV vector that has a tropism to a muscle cell and/or to a muscle stem cell, said method comprising:
transducing a packaging cell according to any one of claims 52-58 with a nucleic acid encoding a construct that is to be delivered into a muscle cell and/or a muscle stem cell where said construct is flanked by AAV ITR sequences;
whereby said packaging cells packages said construct into an AAV capsid to produce an AAV virion that has a tropism to a muscle cell and/or to a muscle stem cell.

61. A method of introducing a nucleic acid construct into a muscle cell or a muscle stem cell in a mammal, said method comprising:
providing an AAV vector according to any one of claims 1-23, wherein said AAV vector comprises a nucleic construct that is to be delivered into a muscle cell and/or muscle stem cell; and
administering said vector to said mammal in an amount effective to transduce a muscle cell and/or a muscle stem cell in said mammal.

62. The method of claim 61, wherein said construct comprises a gene or cDNA selected for the treatment of a skeletal muscle disorder selected from the group consisting of Limb girdle muscular dystrophy (LGMD), myofibrillar myopathy, myotubular myopathy, BODY, inclusion body myopathy, and vacuolar myopathy.

63. The method of claim 62, wherein said skeletal muscle disorder comprises limb girdle muscular dystrophy (LGMD).

64. The method according to any one of claims 62-63, wherein said gene comprise a gene selected from the group consisting of CAPN3, DYSF, TRIM32, ACTA1, MTM1, PAX7, DES, and LAMP2.

65. The method of claim 64, wherein said gene comprises CAPN3.

66. The method of claim 65, wherein said gene comprises a full-length CAPN3 cDNA.

67. The method of claim 66, wherein said gene comprises a UTR-deleted, Kozak-optimized CAPN3 cDNA.

68. The method of claim 61, wherein said construct comprises a nucleic acid that encodes a gene-editing protein.

69. The method of claim 68, wherein said gene editing protein comprises a component of a CRISPR/Cas system, a TALEN, and a zinc finger protein.

70. The method of claim 69, wherein construct encodes a CRISPR protein and a guide RNA.

71. The method of claim 70, wherein said construct encodes an RNA to direct said CRISPR protein to cleave exons 45-55 of the DMD gene.
